(19) United States
(12) Patent Application Publication
(10) Pub. No.: US 2025/0120310 A1
(43) Pub. Date: Apr. 10, 2025

(54) COMPOUND AND ORGANIC LIGHT EMITTING DEVICE COMPRISING THE SAME

(71) Applicant: LG CHEM, LTD., Seoul (KR)

(72) Inventors: Min Woo Jung, Daejeon (KR); Dong Hoon Lee, Daejeon (KR); Jungha Lee, Daejeon (KR); Su Jin Han, Daejeon (KR); Seulchan Park, Daejeon (KR); Sunghyun Hwang, Daejeon (KR); Mintaek Oh, Daejeon (KR)

(21) Appl. No.: 18/567,343

(22) PCT Filed: Sep. 30, 2022
(86) PCT No.: PCT/KR2022/014771

§ 371 Date: Dec. 5, 2023

(30) Foreign Application Priority Data
Oct. 1, 2021 (KR) 10-2021-0130912
Sep. 30, 2022 (KR) 10-2022-0125146

(51) Int. Cl. H10K 85/60; C07D 407/14; H10K 50/12

(52) U.S. Cl. CPC H10K 85/654; C07D 407/14; H10K 85/622; H10K 85/6572; H10K 85/6574; H10K 50/12

(57) ABSTRACT

A compound represented by Chemical Formula 1 and an organic light emitting device including the same are provided.

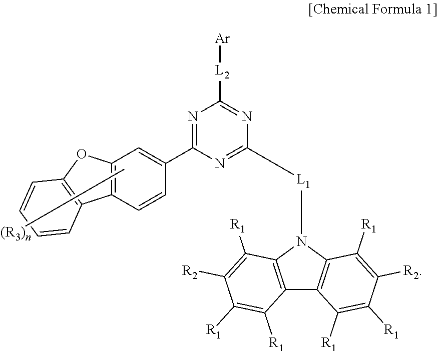

### CROSS-REFERENCE TO RELATED APPLICATION(S)

This application is a National Phase entry pursuant to 35 U.S.C. § 371 of International Application No. PCT/KR2022/014771 filed on Sep. 30, 2022, and claims priority to and the benefit of Korean Patent Application No. 10-2021-0130912 filed on Oct. 1, 2021 and Korean Patent Application No. 10-2022-0125146 filed on Sep. 30, 2022, the disclosures of which are incorporated herein by reference in their entireties.

### FIELD OF DISCLOSURE

The present disclosure relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a need for continuous development of new materials for the organic materials used in the organic light emitting devices as described above.

### RELATED ART

Korean Unexamined Patent Publication No. 10-2000-0051826

### SUMMARY

It is an object of the present disclosure to provide a novel compound and an organic light emitting device comprising the same.

According to an aspect of the present disclosure, there is provided a compound represented by the following Chemical Formula 1:

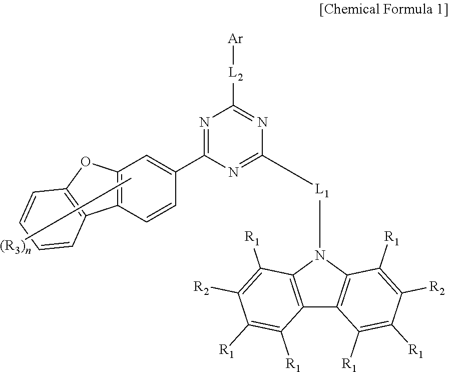

in Chemical Formula 1,
L₁ and L₂ are each independently a single bond, or a substituted or unsubstituted C₆₋₆₀ arylene,
Ar is a substituted or unsubstituted C₆-60 aryl, or a substituted or unsubstituted C₂₋₆₀ heteroaryl containing a heteroatom selected from the group consisting of N, O and S,
each R₁ is independently hydrogen or deuterium, with the proviso that at least one R₁ is deuterium,
each R₂ is independently hydrogen or deuterium,
each R₃ is independently hydrogen or deuterium, and
n is an integer of 0 to 7.

According to another aspect of the present disclosure, there is provided an organic light emitting device comprising: a first electrode; a second electrode provided to face the first electrode; and an organic material layer including one or more layers provided between the first electrode and the second electrode, wherein one or more layers of the organic material layer include the compound represented by Chemical Formula 1.

The above-mentioned compound represented by Chemical Formula 1 can be used as a material for an organic material layer of an organic light emitting device, and can improve the efficiency, achieve low driving voltage and/or improve lifetime characteristics in the organic light emitting device. In particular, the compound represented by Chemical Formula 1 described above can be used as a material for hole injection, hole transport, hole injection and transport, light emission, electron transport, or electron injection.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, a light emitting layer 3, an electron transport layer 7, and a cathode 4.

### DETAILED DESCRIPTION

Hereinafter, embodiments of the present disclosure will be described in more detail to facilitate understanding of the invention.

As used herein, the notation

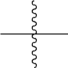

and [symbol] mean a bond linked to another substituent group.

As used herein, the term “substituted or unsubstituted” means being unsubstituted or substituted with one or more substituents selected from the group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent from the above substituent group which is further substituted by one or more selected from the above substituent group.

In the present disclosure, the carbon number of a carbonyl group is not particularly limited, but is preferably 1 to 40. Specifically, the carbonyl group may be a substituent group having the following structural formulas, but is not limited thereto.

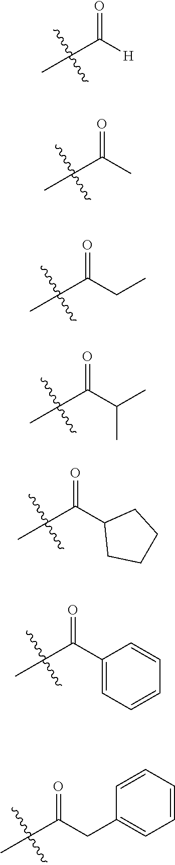

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent group having the following structural formulas, but is not limited thereto.

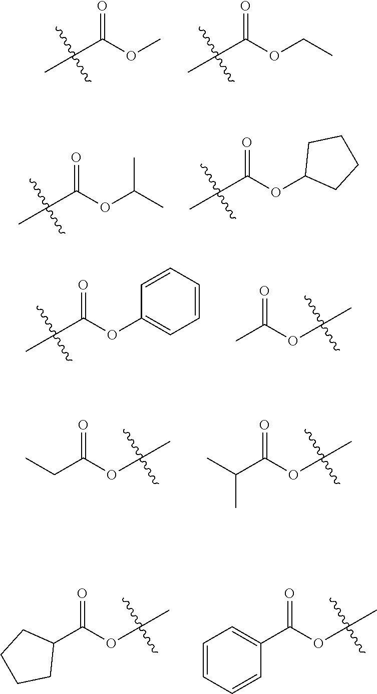

In the present disclosure, the carbon number of an imide group is not particularly limited, but is preferably 1 to 25. Specifically, the imide group may be a substituent group having the following structural formulas, but is not limited thereto.

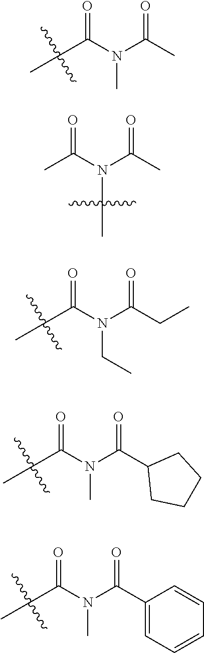

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group may be substituted, and two substituents may be connected to each other to form a spiro structure. In the case where the fluorenyl group is substituted,

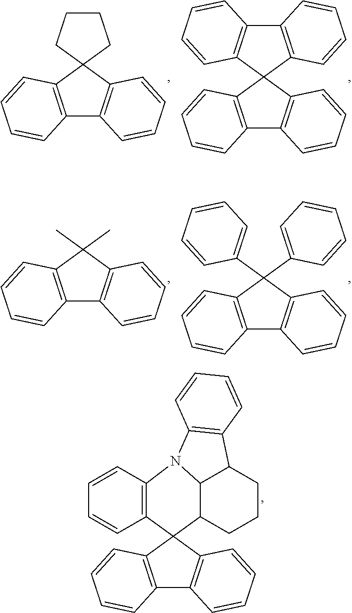

and the like can be formed. However, the structure is not limited thereto

In the present disclosure, a heterocyclic group is a heterocyclic group containing at least one of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the above-mentioned examples of the aryl group. In the present disclosure, the alkyl group in the aralkyl group, the alkylaryl group and the alkylamine group is the same as the above-mentioned examples of the alkyl group. In the present disclosure, the heteroaryl in the heteroarylamine may be applied to the above-mentioned description of the heterocyclic group. In the present disclosure, the alkenyl group in the aralkenyl group is the same as the above-mentioned examples of the alkenyl group. In the present disclosure, the above-mentioned description of the aryl group may be applied except that the arylene is a divalent group. In the present disclosure, the above-mentioned description of the heterocyclic group may be applied except that the heteroarylene is a divalent group. In the present disclosure, the above-mentioned description of the aryl group or cycloalkyl group may be applied except that the hydrocarbon ring is not a monovalent group but formed by combining two substituent groups. In the present disclosure, the above-mentioned description of the heterocyclic group may be applied, except that the heterocyclic group is not a monovalent group but formed by combining two substituent groups.

### Compound

The present disclosure provides the compound represented by Chemical Formula 1.

Preferably, L₁ may be a single bond or phenylene.

Preferably, L₂ is a single bond, phenylene, or naphthylene.

Preferably, Ar is phenyl, biphenylyl, terphenylyl, naphthyl, phenanthrenyl, triphenylenyl, dibenzofuranyl, dibenzothiophenyl, carbazol-9-yl, 9-phenylcarbazolyl, or dimethylfluorenyl, each of which is unsubstituted or substituted with at least one deuterium, cyano, or halogen.

Preferably, at least two R₁s are deuterium.

More preferably, 2 or more, 3 or more, 4 or more, or 5 or more, and 6 or less R₁s are deuterium.

Preferably, all R₂s are hydrogen, or all R₂s are deuterium.

Preferably, R₃ is deuterium, and n is 0, 4, 6, or 7.

More preferably, R₃ is deuterium and n is 0, or 2 or more, 3 or more, 4 or more, 5 or more, or 6 or more, and 7 or less.

Preferably, the substituent

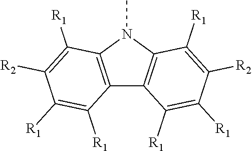

is one selected from the following:

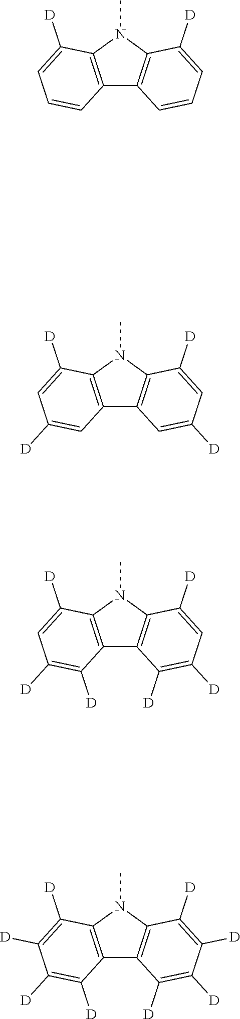

Representative examples of the compound represented by Chemical Formula 1 is as follows:

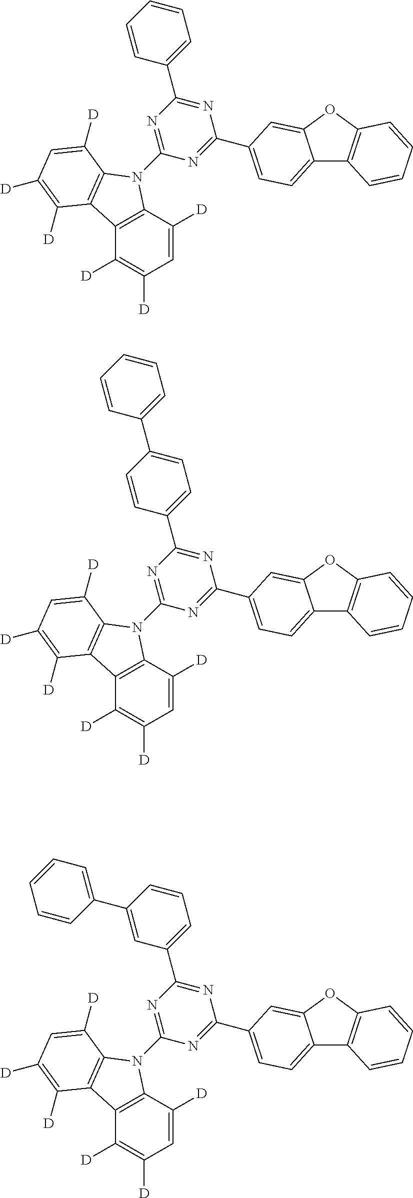 [SMILES: [2H]c1cc([2H])c2c(c1[2H])c1c([2H])c([2H])cc([2H])c1n2-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3ccccc3)cc2)nc(-c2ccc3c(c2)oc2ccccc23)n1 ; [2H]c1cc([2H])c2c(c1[2H])c1c([2H])c([2H])cc([2H])c1n2-c1nc(-c2cccc(-c3ccccc3)c2)nc(-c2ccc3c(c2)oc2ccccc23)n1]

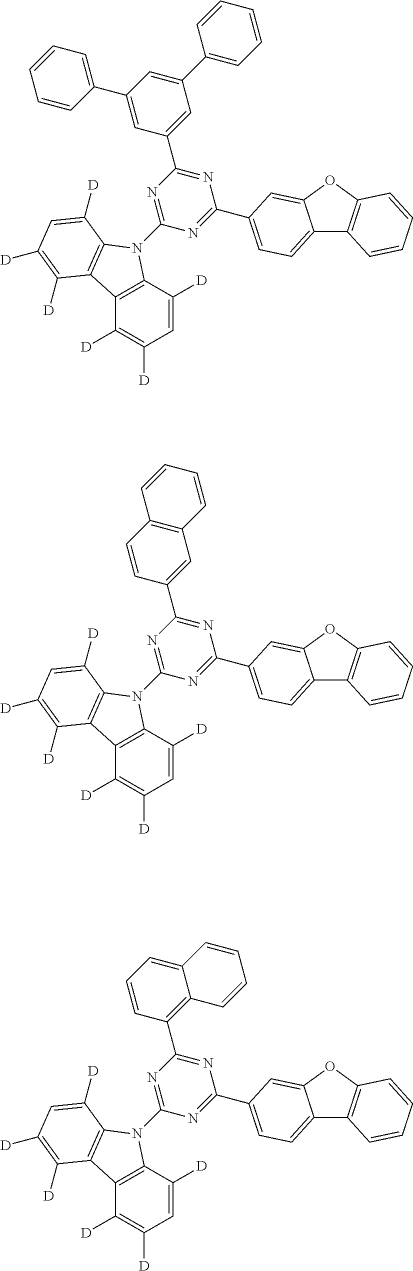 [SMILES: [2H]c1cc([2H])c2c(c1[2H])c1c([2H])c([2H])cc([2H])c1n2-c1nc(-c2cc(-c3ccccc3)cc(-c3ccccc3)c2)nc(-c2ccc3c(c2)oc2ccccc23)n1 ; [2H]c1cc([2H])c2c(c1[2H])c1c([2H])c([2H])cc([2H])c1n2-c1nc(-c2ccc3ccccc3c2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2cccc3ccccc23)n1]

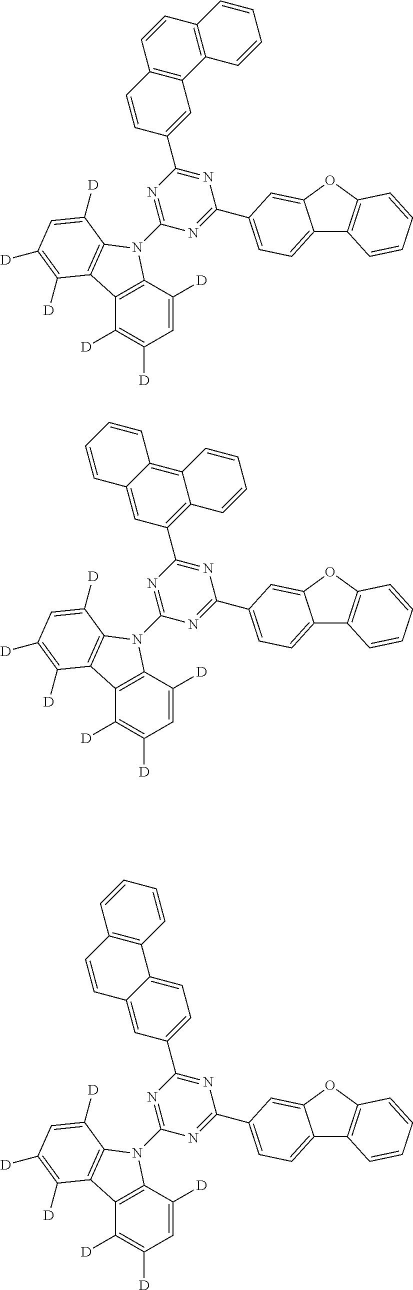 [SMILES: [2H]c1cc([2H])c2c(c1[2H])c1c([2H])c([2H])cc([2H])c1n2-c1nc(-c2ccc3c(c2)oc2ccccc23)nc(-c2ccc3ccc4ccccc4c3c2)n1 ; [2H]c1cc([2H])c2c(c1[2H])c1c([2H])c([2H])cc([2H])c1n2-c1nc(-c2ccc3c(c2)oc2ccccc23)nc(-c2cc3ccccc3c3ccccc23)n1 ; [2H]c1cc([2H])c2c(c1[2H])c1c([2H])c([2H])cc([2H])c1n2-c1nc(-c2ccc3c(ccc4ccccc43)c2)nc(-c2ccc3c(c2)oc2ccccc23)n1]

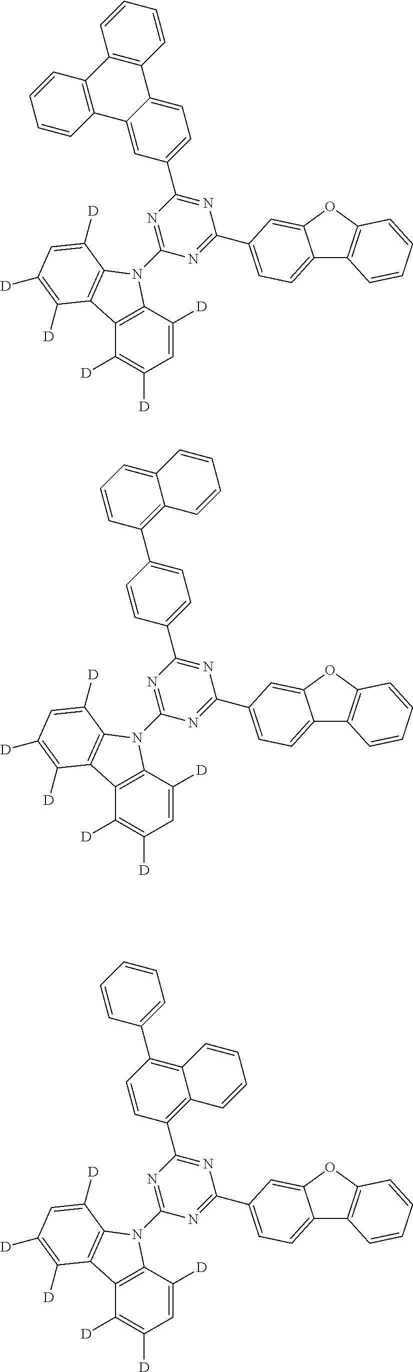 [SMILES: [2H]c1cc([2H])c2c(c1[2H])c1c([2H])c([2H])cc([2H])c1n2-c1nc(-c2ccc3c(c2)oc2ccccc23)nc(-c2ccc3c4ccccc4c4ccccc4c3c2)n1 ; [2H]c1cc([2H])c2c(c1[2H])c1c([2H])c([2H])cc([2H])c1n2-c1nc(-c2ccc(-c3cccc4ccccc34)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc(-c3ccccc3)c3ccccc23)n1]

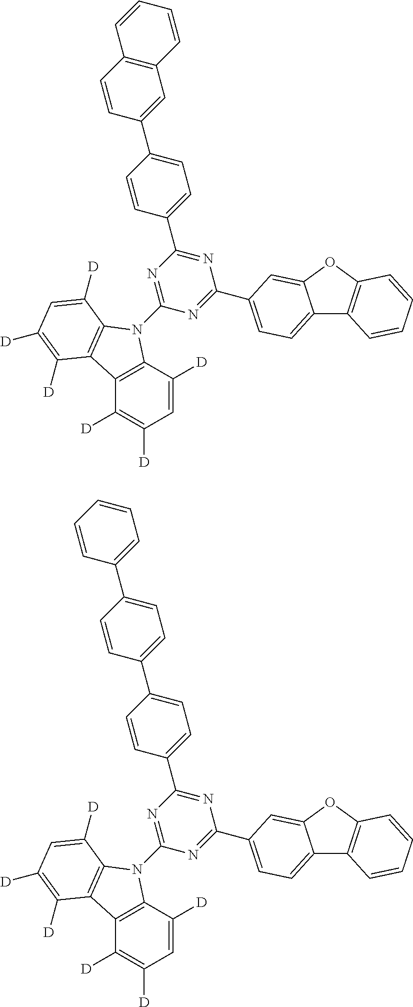 [SMILES: [2H]c1cc([2H])c2c(c1[2H])c1c([2H])c([2H])cc([2H])c1n2-c1nc(-c2ccc(-c3ccc4ccccc4c3)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3ccc(-c4ccccc4)cc3)cc2)nc(-c2ccc3c(c2)oc2ccccc23)n1]

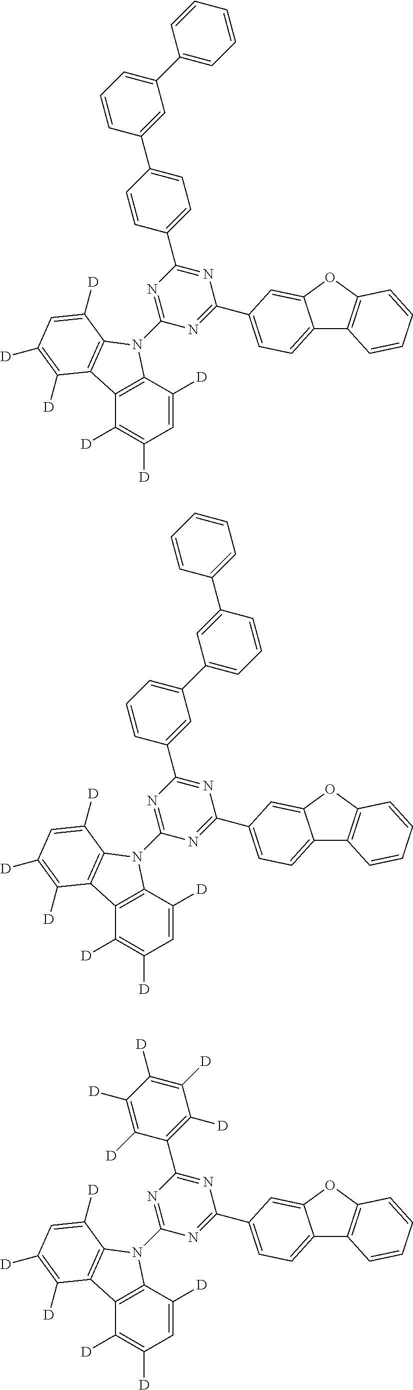 [SMILES: [2H]c1cc([2H])c2c(c1[2H])c1c([2H])c([2H])cc([2H])c1n2-c1nc(-c2ccc(-c3cccc(-c4ccccc4)c3)cc2)nc(-c2ccc3c(c2)oc2ccccc23)n1 ; [2H]c1cc([2H])c2c(c1[2H])c1c([2H])c([2H])cc([2H])c1n2-c1nc(-c2cccc(-c3cccc(-c4ccccc4)c3)c2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2c([2H])c([2H])c([2H])c([2H])c2[2H])n1]

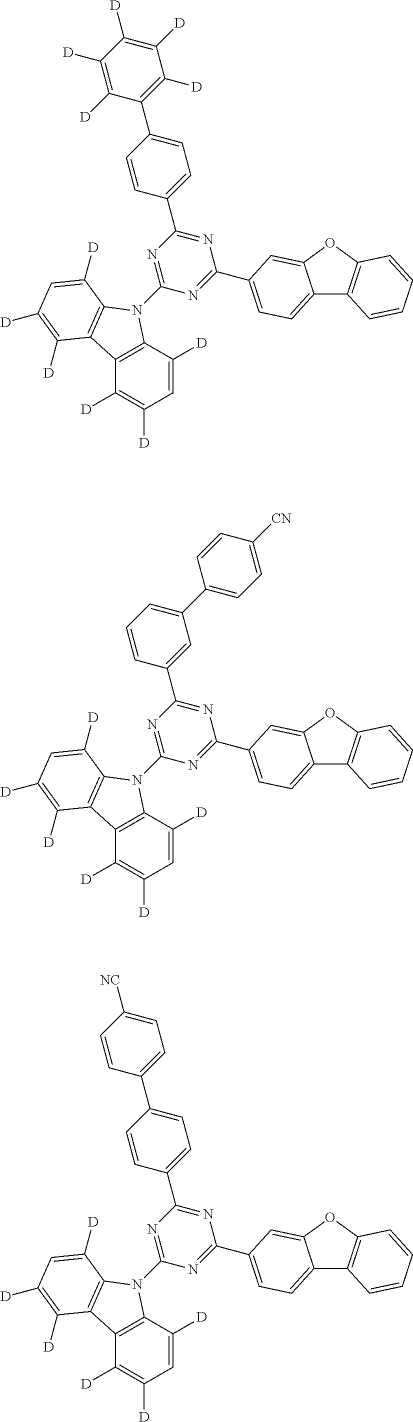 [SMILES: [2H]c1cc([2H])c2c(c1[2H])c1c([2H])c([2H])cc([2H])c1n2-c1nc(-c2ccc(-c3c([2H])c([2H])c([2H])c([2H])c3[2H])cc2)nc(-c2ccc3c(c2)oc2ccccc23)n1 ; [2H]c1cc([2H])c2c(c1[2H])c1c([2H])c([2H])cc([2H])c1n2-c1nc(-c2cccc(-c3ccc(C#N)cc3)c2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3ccc(C#N)cc3)cc2)nc(-c2ccc3c(c2)oc2ccccc23)n1]

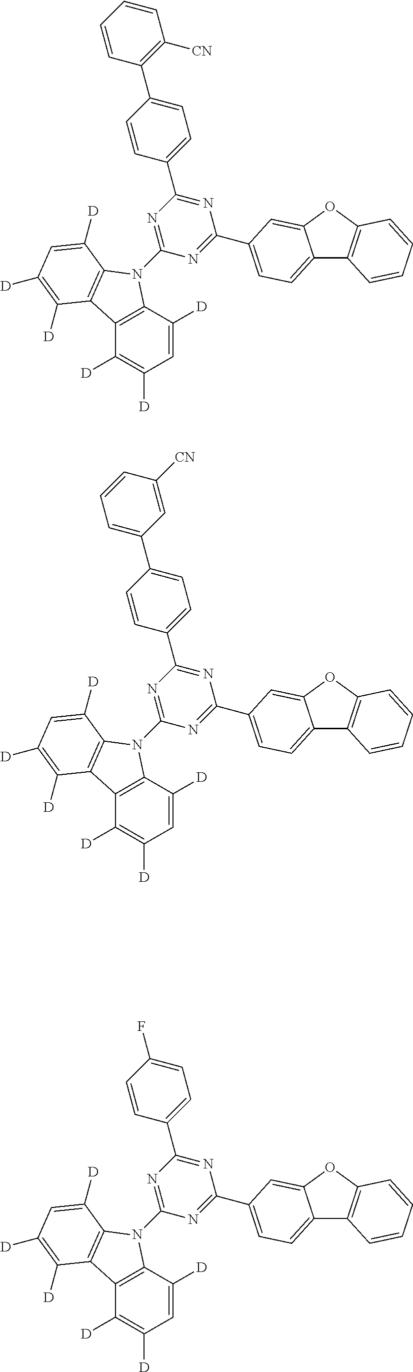 [SMILES: [2H]c1cc([2H])c2c(c1[2H])c1c([2H])c([2H])cc([2H])c1n2-c1nc(-c2ccc(-c3ccccc3C#N)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3cccc(C#N)c3)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc(F)cc2)nc(-c2ccc3c(c2)oc2ccccc23)n1]

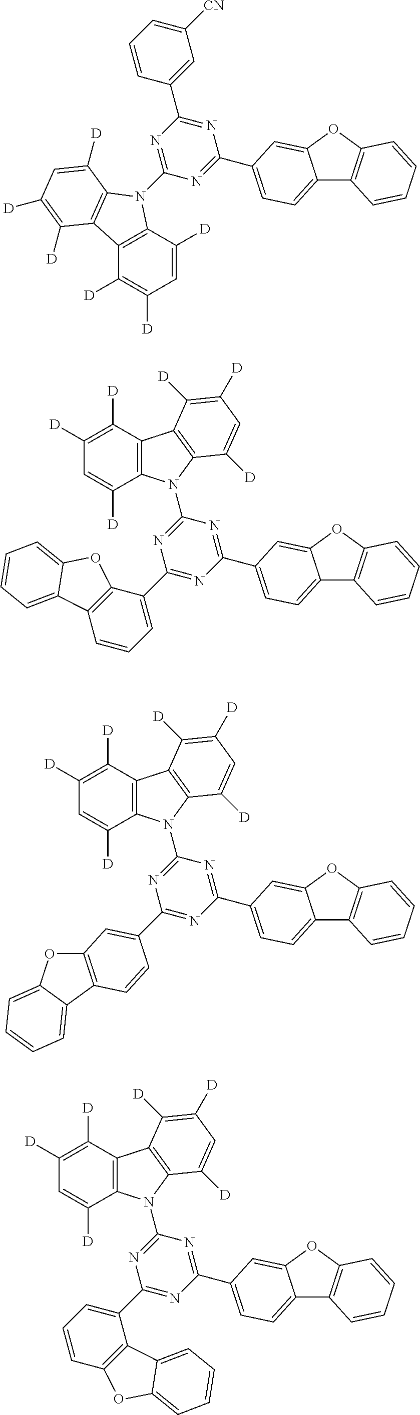 [SMILES: [2H]c1cc([2H])c2c(c1[2H])c1c([2H])c([2H])cc([2H])c1n2-c1nc(-c2cccc(C#N)c2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2cccc3c2oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2cccc3oc4ccccc4c23)n1]

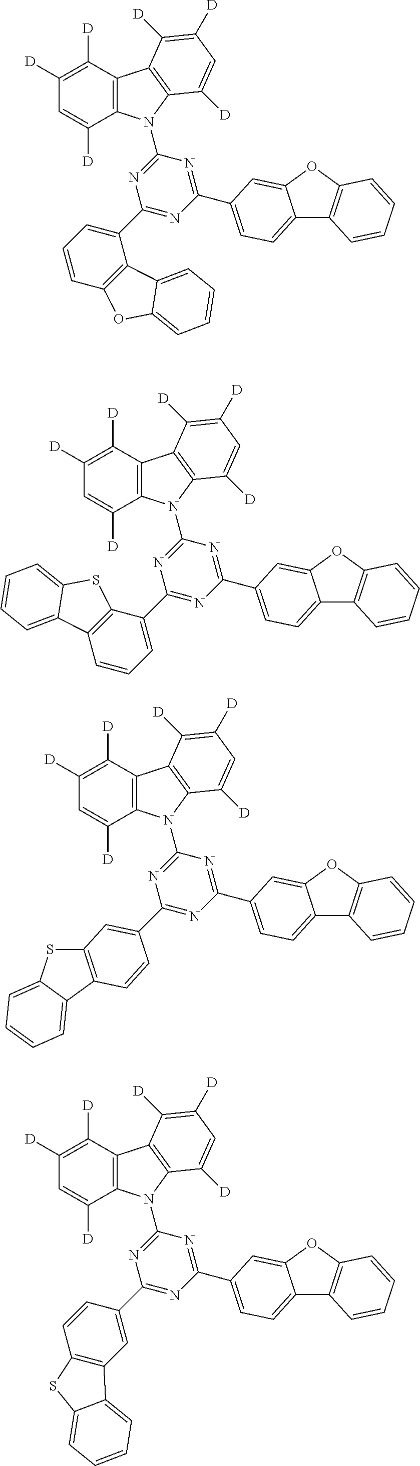 [SMILES: [2H]c1cc([2H])c2c(c1[2H])c1c([2H])c([2H])cc([2H])c1n2-c1nc(-c2ccc3c(c2)oc2ccccc23)nc(-c2cccc3oc4ccccc4c23)n1 ; [2H]c1cc([2H])c2c(c1[2H])c1c([2H])c([2H])cc([2H])c1n2-c1nc(-c2ccc3c(c2)oc2ccccc23)nc(-c2cccc3c2sc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc3c(c2)sc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc3sc4ccccc4c3c2)n1]

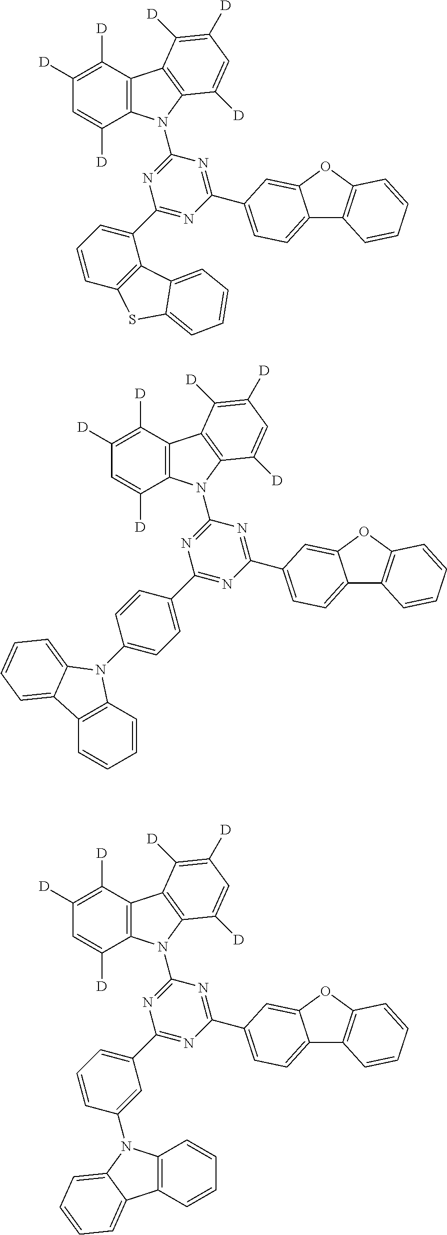

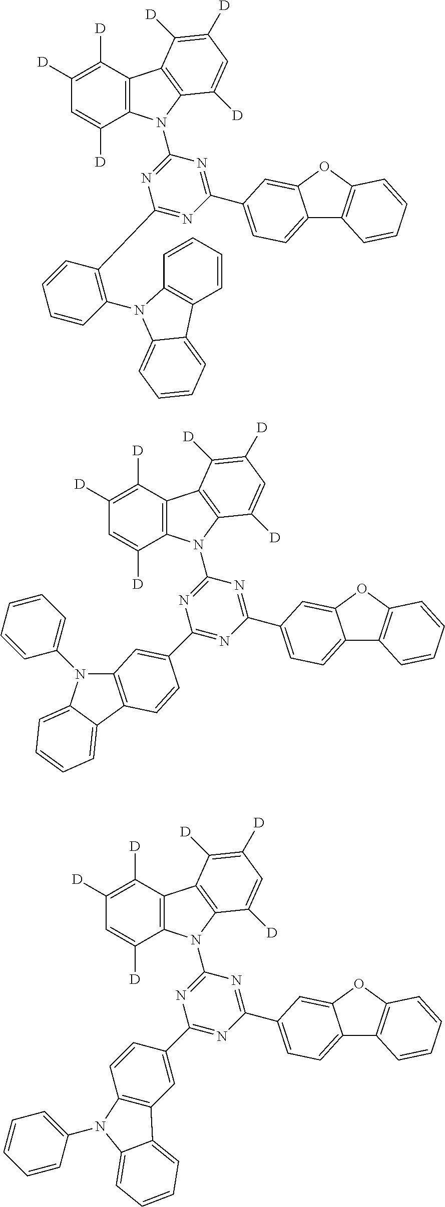

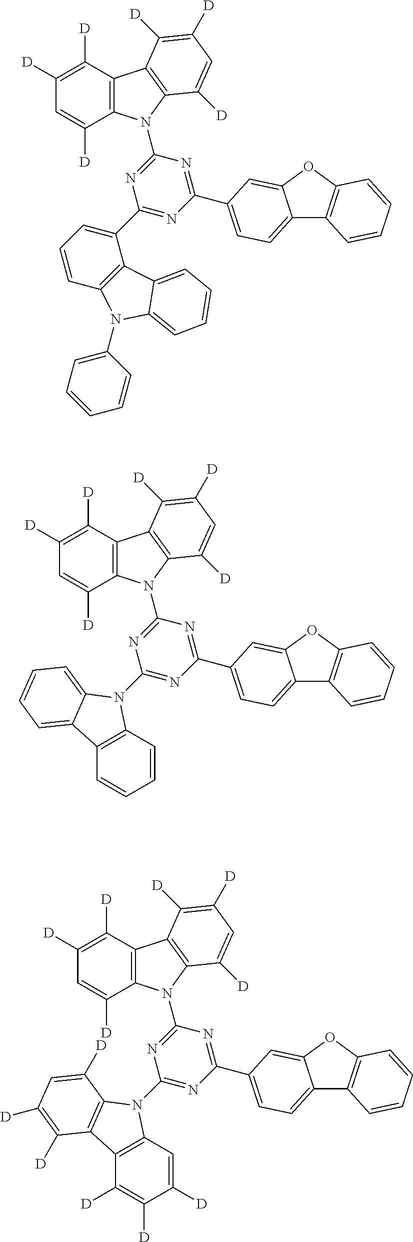 [SMILES: [2H]c1cc([2H])c2c(c1[2H])c1c([2H])c([2H])cc([2H])c1n2-c1nc(-c2ccc3c(c2)oc2ccccc23)nc(-c2cccc3c2c2ccccc2n3-c2ccccc2)n1 ; [2H]c1cc([2H])c2c(c1[2H])c1c([2H])c([2H])cc([2H])c1n2-c1nc(-c2ccc3c(c2)oc2ccccc23)nc(-n2c3ccccc3c3ccccc32)n1 ; [2H]c1cc2c(c([2H])c1[2H])c1c([2H])c([2H])cc([2H])c1n2-c1nc(-c2ccc3c(c2)oc2ccccc23)nc(-n2c3c([2H])cc([2H])c([2H])c3c3c([2H])c([2H])cc([2H])c32)n1]

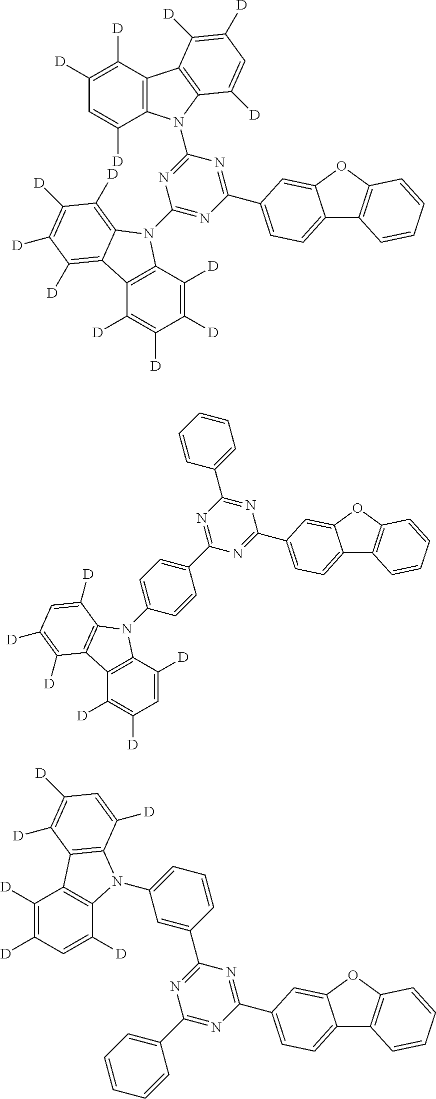 [SMILES: [2H]c1cc([2H])c2c(c1[2H])c1c([2H])c([2H])cc([2H])c1n2-c1nc(-c2ccc3c(c2)oc2ccccc23)nc(-n2c3c([2H])c([2H])c([2H])c([2H])c3c3c([2H])c([2H])c([2H])c([2H])c32)n1 ; [2H]c1cc([2H])c2c(c1[2H])c1c([2H])c([2H])cc([2H])c1n2-c1ccc(-c2nc(-c3ccccc3)nc(-c3ccc4c(c3)oc3ccccc34)n2)cc1 ; [2H]c1cc([2H])c2c(c1[2H])c1c([2H])c([2H])cc([2H])c1n2-c1cccc(-c2nc(-c3ccccc3)nc(-c3ccc4c(c3)oc3ccccc34)n2)c1]

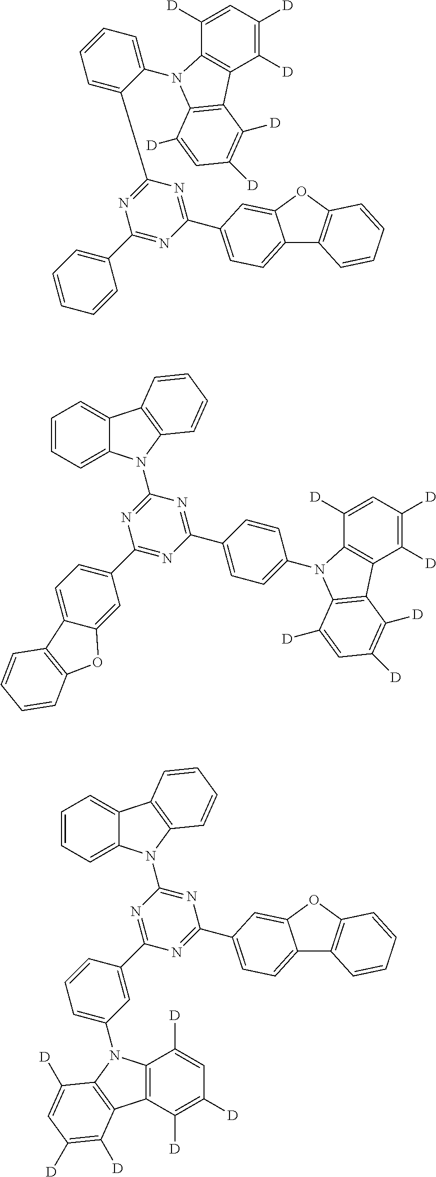 [SMILES: [2H]c1cc([2H])c2c(c1[2H])c1c([2H])c([2H])cc([2H])c1n2-c1ccccc1-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2nc(-c3ccc4c(c3)oc3ccccc34)nc(-n3c4ccccc4c4ccccc43)n2)cc1 ; [2H]c1cc([2H])c2c(c1[2H])c1c([2H])c([2H])cc([2H])c1n2-c1cccc(-c2nc(-c3ccc4c(c3)oc3ccccc34)nc(-n3c4ccccc4c4ccccc43)n2)c1]

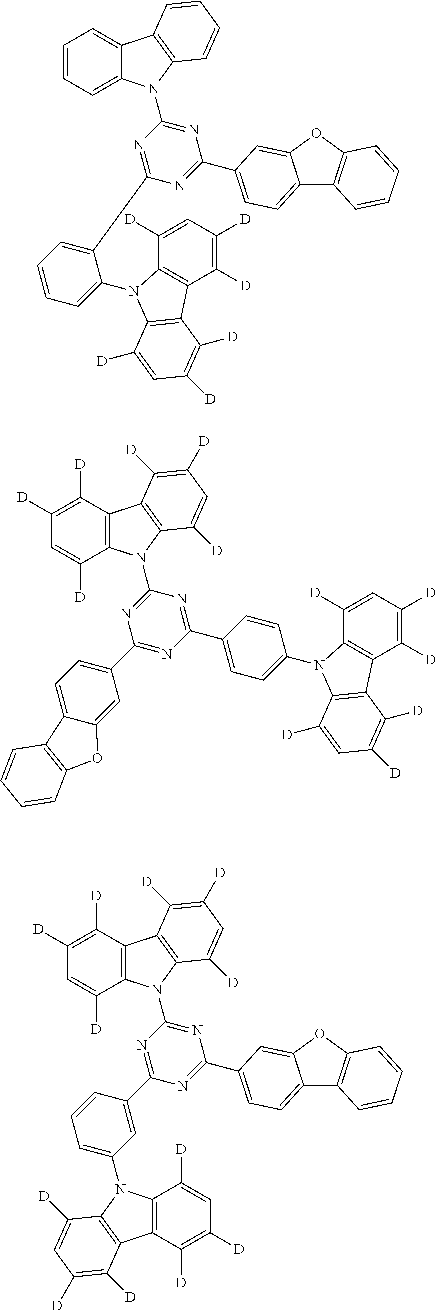

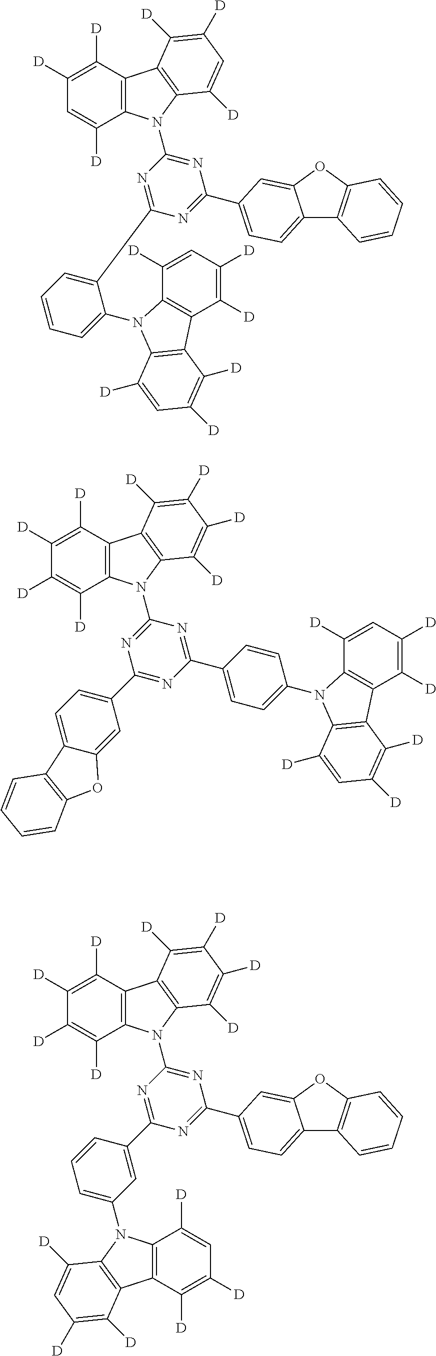

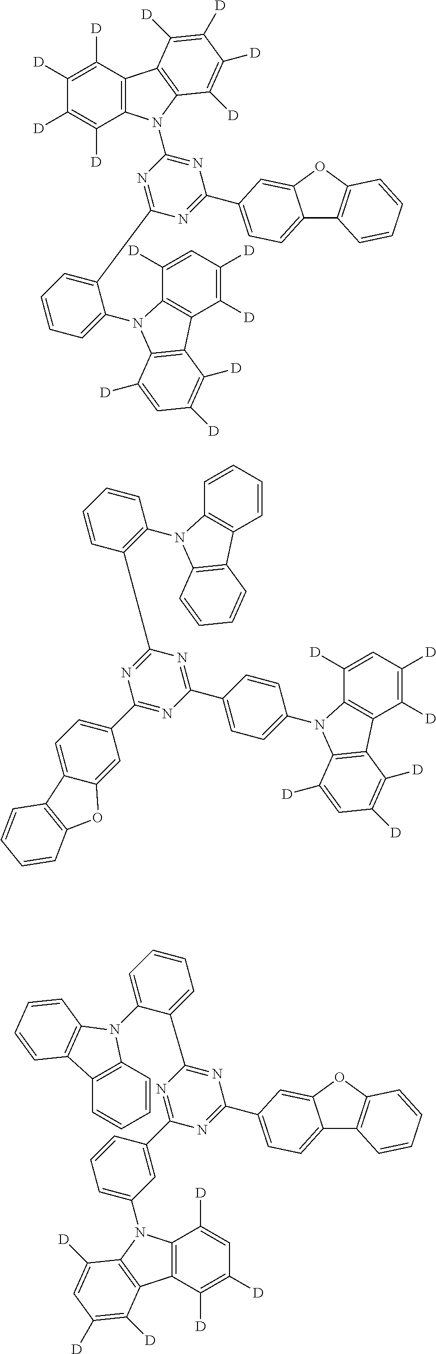

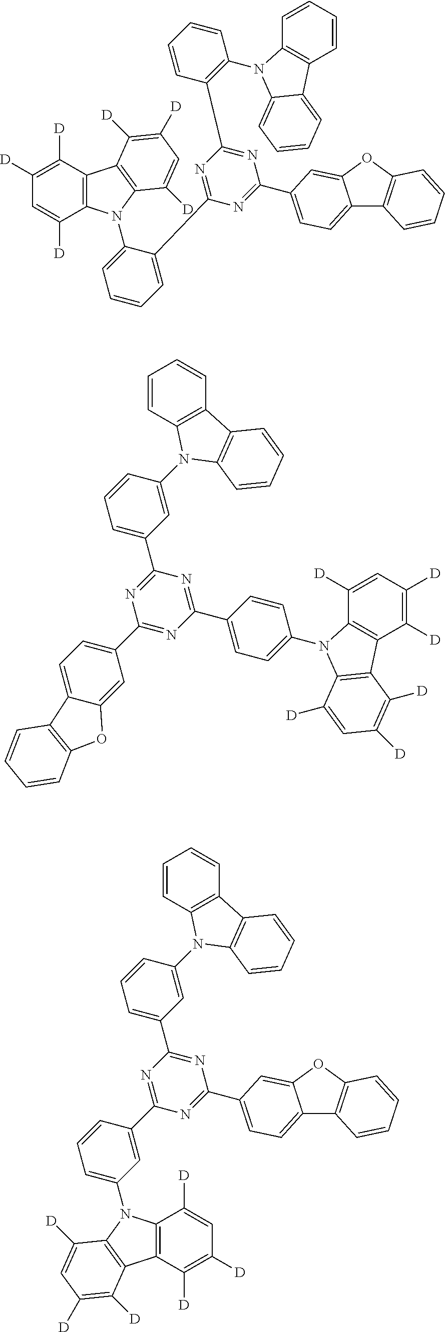 [SMILES: [2H]c1cc([2H])c2c(c1[2H])c1c([2H])c([2H])cc([2H])c1n2-c1ccccc1-c1nc(-c2ccc3c(c2)oc2ccccc23)nc(-c2ccccc2-n2c3ccccc3c3ccccc32)n1 ; [2H]c1cc([2H])c2c(c1[2H])c1c([2H])c([2H])cc([2H])c1n2-c1ccc(-c2nc(-c3cccc(-n4c5ccccc5c5ccccc54)c3)nc(-c3ccc4c(c3)oc3ccccc34)n2)cc1 ; [2H]c1cc([2H])c2c(c1[2H])c1c([2H])c([2H])cc([2H])c1n2-c1cccc(-c2nc(-c3cccc(-n4c5ccccc5c5ccccc54)c3)nc(-c3ccc4c(c3)oc3ccccc34)n2)c1]

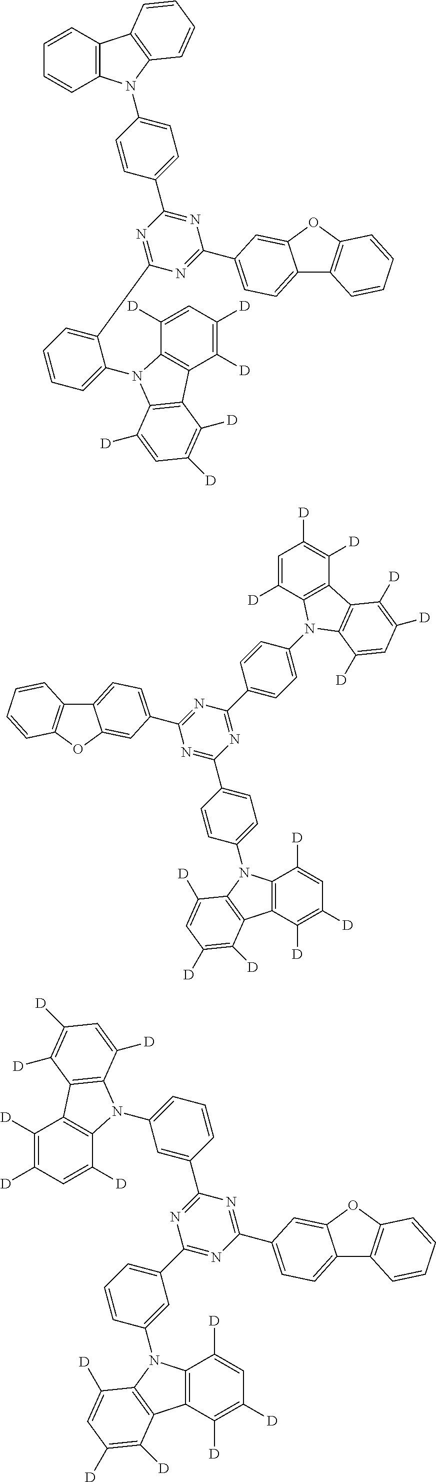 [SMILES: [2H]c1cc([2H])c2c(c1[2H])c1c([2H])c([2H])cc([2H])c1n2-c1ccccc1-c1nc(-c2ccc(-n3c4ccccc4c4ccccc43)cc2)nc(-c2ccc3c(c2)oc2ccccc23)n1 ; [2H]c1cc([2H])c2c(c1[2H])c1c([2H])c([2H])cc([2H])c1n2-c1ccc(-c2nc(-c3ccc(-n4c5c([2H])cc([2H])c([2H])c5c5c([2H])c([2H])cc([2H])c54)cc3)nc(-c3ccc4c(c3)oc3ccccc34)n2)cc1 ; [2H]c1cc([2H])c2c(c1[2H])c1c([2H])c([2H])cc([2H])c1n2-c1cccc(-c2nc(-c3cccc(-n4c5c([2H])cc([2H])c([2H])c5c5c([2H])c([2H])cc([2H])c54)c3)nc(-c3ccc4c(c3)oc3ccccc34)n2)c1]

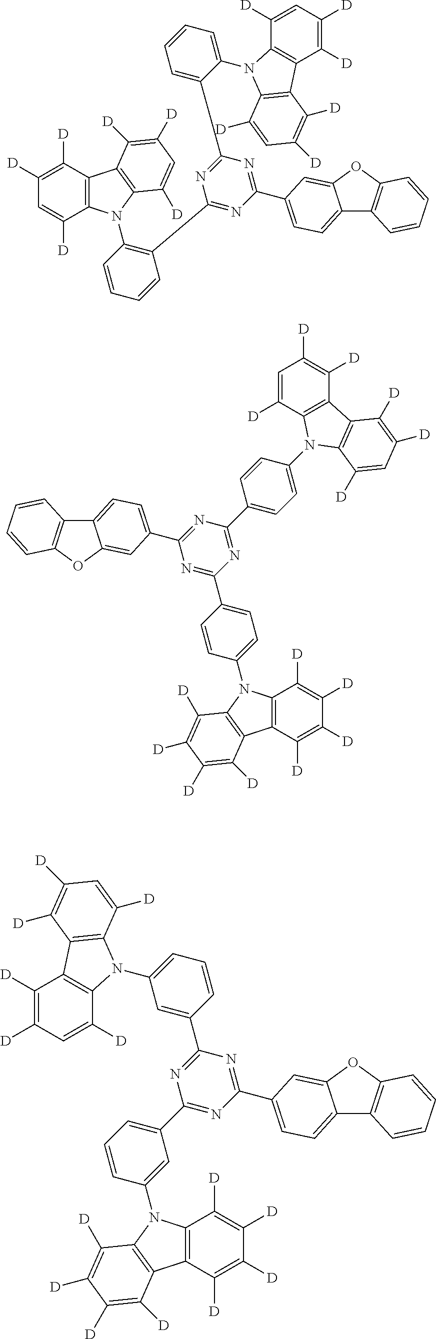

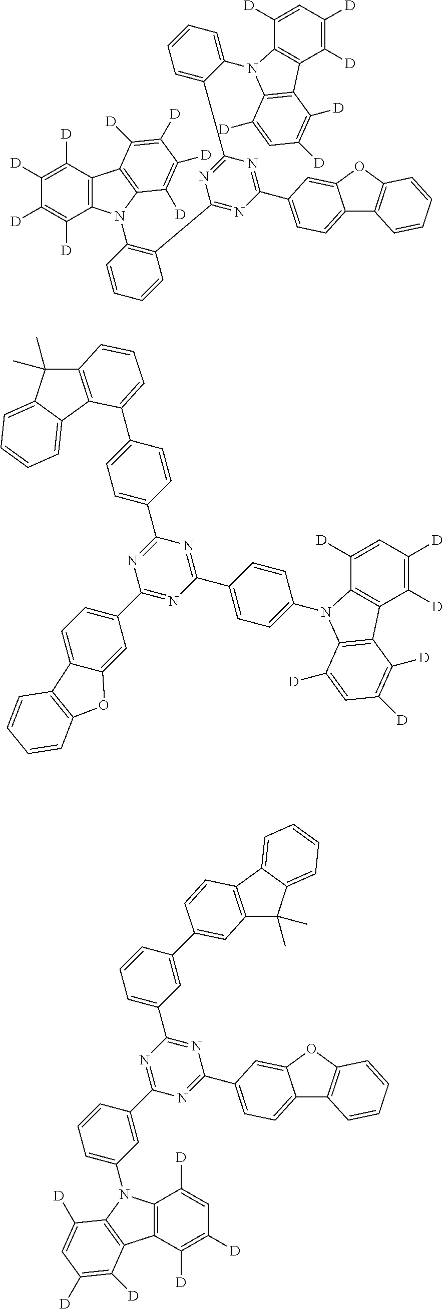 [SMILES: [2H]c1cc([2H])c2c(c1[2H])c1c([2H])c([2H])cc([2H])c1n2-c1ccccc1-c1nc(-c2ccc3c(c2)oc2ccccc23)nc(-c2ccccc2-n2c3c([2H])c([2H])c([2H])c([2H])c3c3c([2H])c([2H])c([2H])c([2H])c32)n1 ; [2H]c1cc([2H])c2c(c1[2H])c1c([2H])c([2H])cc([2H])c1n2-c1ccc(-c2nc(-c3ccc(-c4cccc5c4-c4ccccc4C5(C)C)cc3)nc(-c3ccc4c(c3)oc3ccccc34)n2)cc1 ; [2H]c1cc([2H])c2c(c1[2H])c1c([2H])c([2H])cc([2H])c1n2-c1cccc(-c2nc(-c3cccc(-c4ccc5c(c4)C(C)(C)c4ccccc4-5)c3)nc(-c3ccc4c(c3)oc3ccccc34)n2)c1]

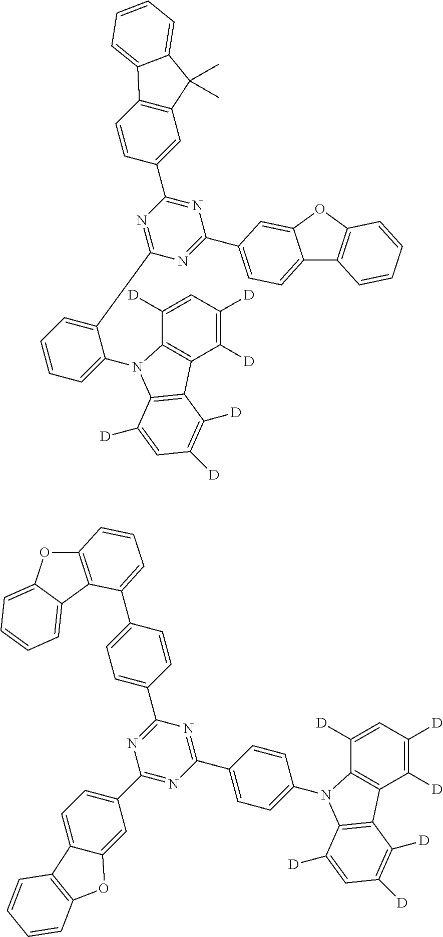 [SMILES: [2H]c1cc([2H])c2c(c1[2H])c1c([2H])c([2H])cc([2H])c1n2-c1ccccc1-c1nc(-c2ccc3c(c2)C(C)(C)c2ccccc2-3)nc(-c2ccc3c(c2)oc2ccccc23)n1 ; [2H]c1cc([2H])c2c(c1[2H])c1c([2H])c([2H])cc([2H])c1n2-c1ccc(-c2nc(-c3ccc(-c4cccc5oc6ccccc6c45)cc3)nc(-c3ccc4c(c3)oc3ccccc34)n2)cc1]

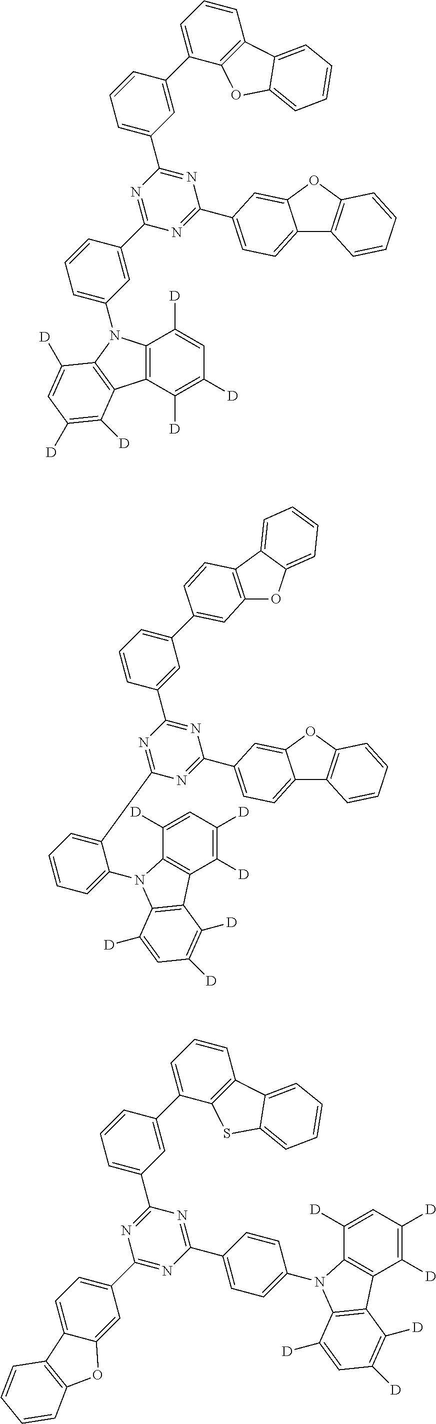 [SMILES: [2H]c1cc([2H])c2c(c1[2H])c1c([2H])c([2H])cc([2H])c1n2-c1cccc(-c2nc(-c3cccc(-c4cccc5c4oc4ccccc45)c3)nc(-c3ccc4c(c3)oc3ccccc34)n2)c1 ; [2H]c1cc([2H])c2c(c1[2H])c1c([2H])c([2H])cc([2H])c1n2-c1ccccc1-c1nc(-c2cccc(-c3ccc4c(c3)oc3ccccc34)c2)nc(-c2ccc3c(c2)oc2ccccc23)n1 ; [2H]c1cc([2H])c2c(c1[2H])c1c([2H])c([2H])cc([2H])c1n2-c1ccc(-c2nc(-c3cccc(-c4cccc5c4sc4ccccc45)c3)nc(-c3ccc4c(c3)oc3ccccc34)n2)cc1]

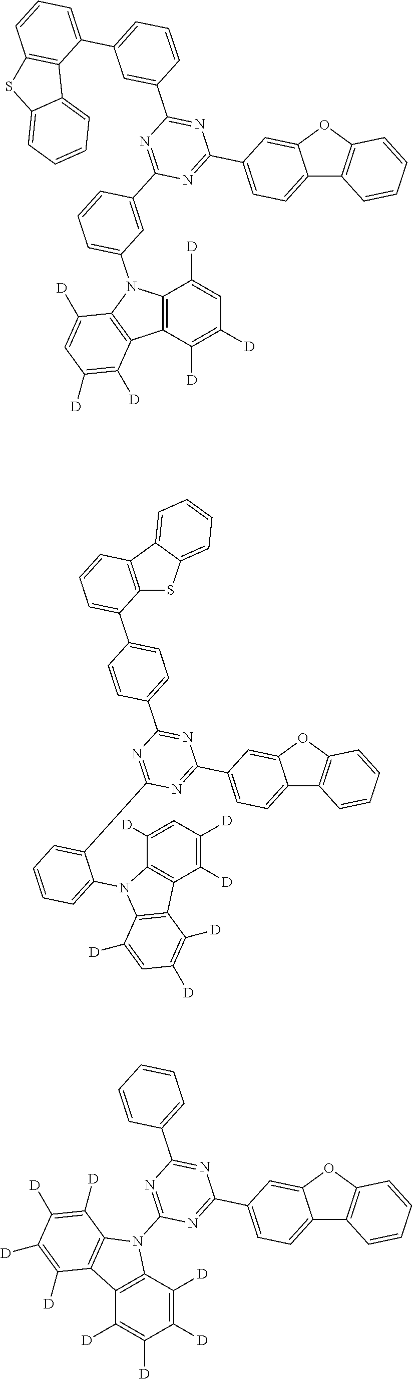 [SMILES: [2H]c1cc([2H])c2c(c1[2H])c1c([2H])c([2H])cc([2H])c1n2-c1cccc(-c2nc(-c3cccc(-c4cccc5sc6ccccc6c45)c3)nc(-c3ccc4c(c3)oc3ccccc34)n2)c1 ; [2H]c1cc([2H])c2c(c1[2H])c1c([2H])c([2H])cc([2H])c1n2-c1ccccc1-c1nc(-c2ccc(-c3cccc4c3sc3ccccc34)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccccc2)nc(-c2ccc3c(c2)oc2ccccc23)n1]

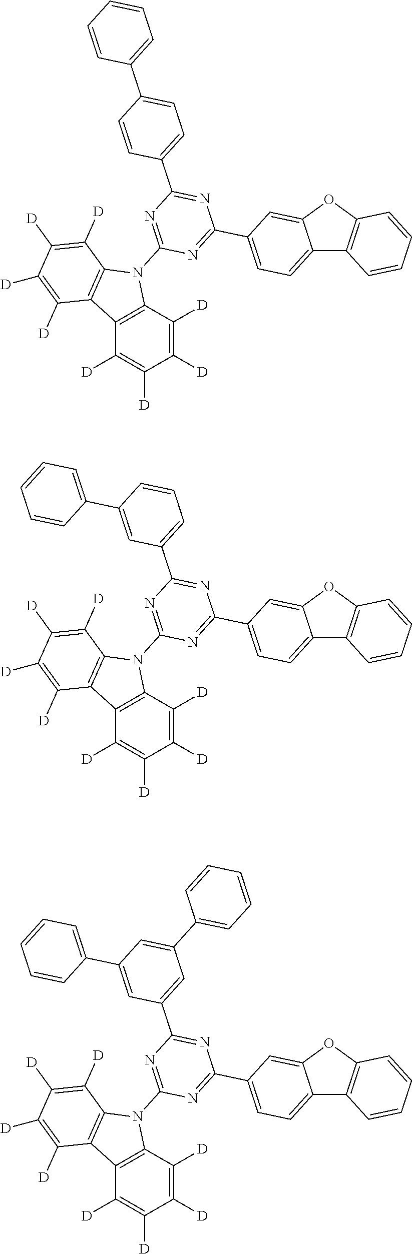 [SMILES: [2H]c1c([2H])c([2H])c2c(c1[2H])c1c([2H])c([2H])c([2H])c([2H])c1n2-c1nc(-c2ccc(-c3ccccc3)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2cccc(-c3ccccc3)c2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2cc(-c3ccccc3)cc(-c3ccccc3)c2)nc(-c2ccc3c(c2)oc2ccccc23)n1]

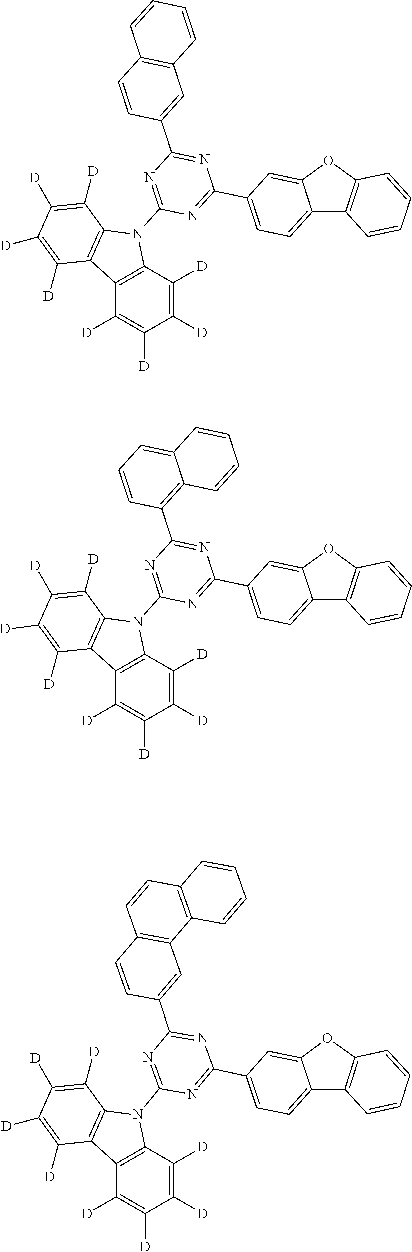 [SMILES: [2H]c1c([2H])c([2H])c2c(c1[2H])c1c([2H])c([2H])c([2H])c([2H])c1n2-c1nc(-c2ccc3ccccc3c2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2cccc3ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2ccc3ccc4ccccc4c3c2)n1]

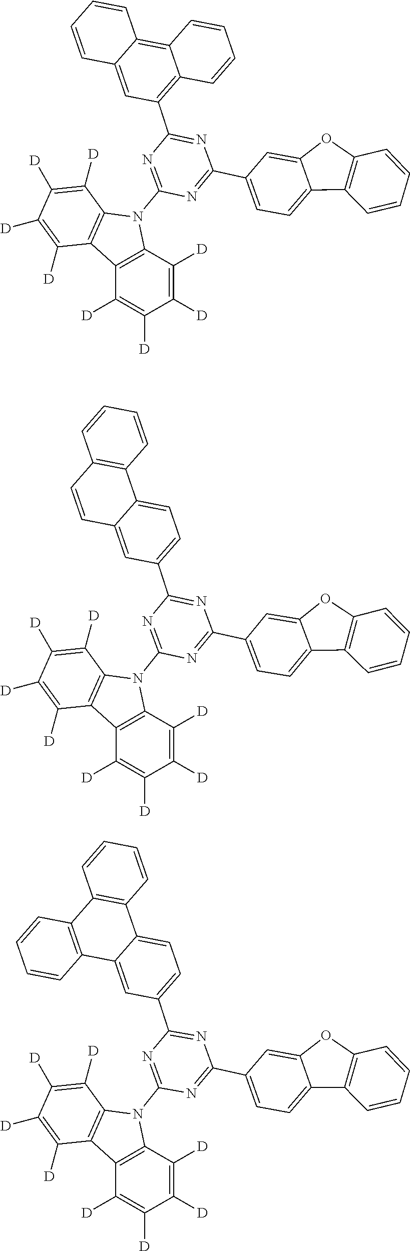

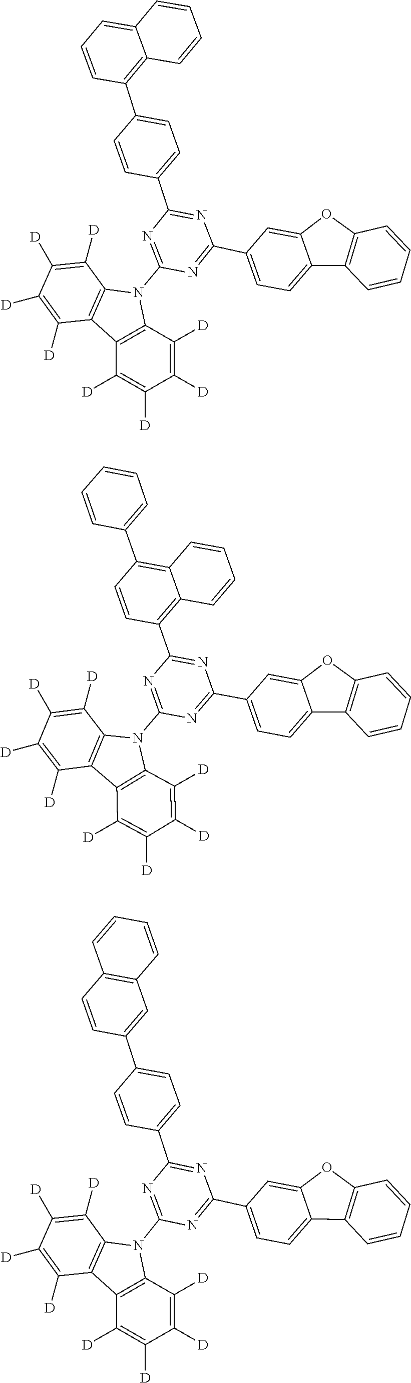 [SMILES: [2H]c1c([2H])c([2H])c2c(c1[2H])c1c([2H])c([2H])c([2H])c([2H])c1n2-c1nc(-c2ccc(-c3cccc4ccccc34)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2ccc(-c3ccccc3)c3ccccc23)n1 ; [2H]c1c([2H])c([2H])c2c(c1[2H])c1c([2H])c([2H])c([2H])c([2H])c1n2-c1nc(-c2ccc(-c3ccc4ccccc4c3)cc2)nc(-c2ccc3c(c2)oc2ccccc23)n1]

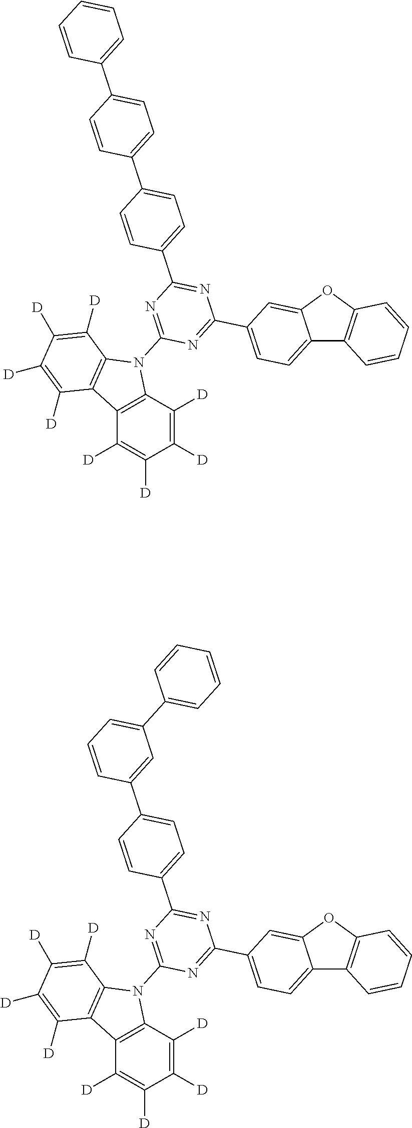 [SMILES: [2H]c1c([2H])c([2H])c2c(c1[2H])c1c([2H])c([2H])c([2H])c([2H])c1n2-c1nc(-c2ccc(-c3ccc(-c4ccccc4)cc3)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc(-c3cccc(-c4ccccc4)c3)cc2)nc(-c2ccc3c(c2)oc2ccccc23)n1]

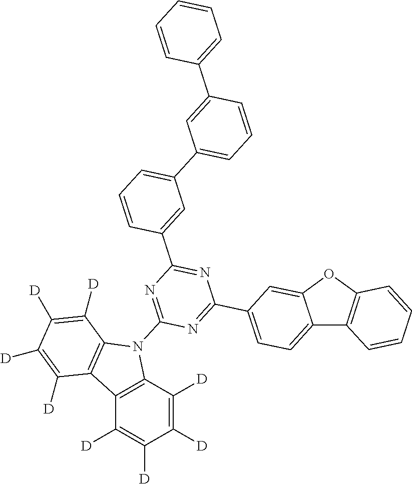 [SMILES: [2H]c1c([2H])c([2H])c2c(c1[2H])c1c([2H])c([2H])c([2H])c([2H])c1n2-c1nc(-c2cccc(-c3cccc(-c4ccccc4)c3)c2)nc(-c2ccc3c(c2)oc2ccccc23)n1]

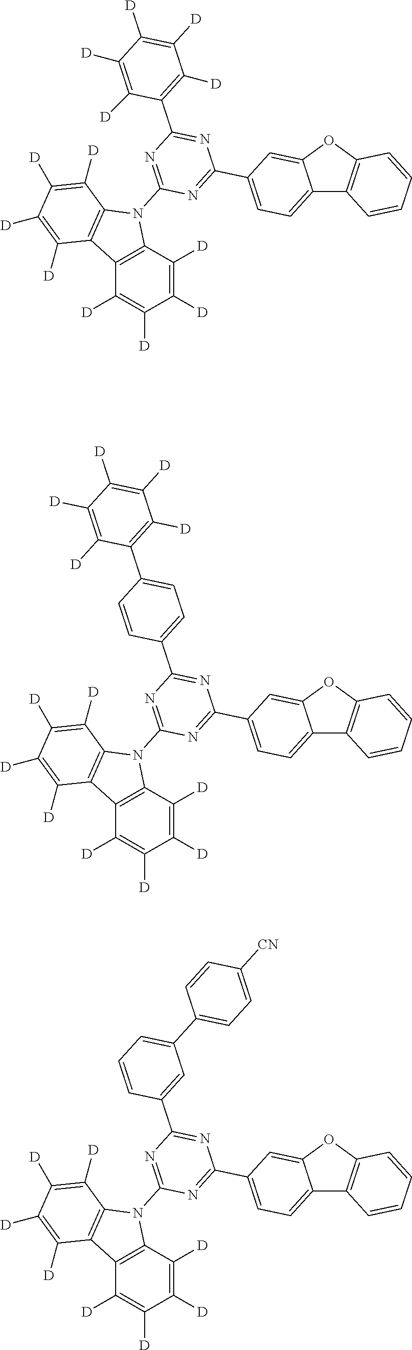 [SMILES: [2H]c1c([2H])c([2H])c(-c2nc(-c3ccc4c(c3)oc3ccccc34)nc(-n3c4c([2H])c([2H])c([2H])c([2H])c4c4c([2H])c([2H])c([2H])c([2H])c43)n2)c([2H])c1[2H] ; [2H]c1c([2H])c([2H])c(-c2ccc(-c3nc(-c4ccc5c(c4)oc4ccccc45)nc(-n4c5c([2H])c([2H])c([2H])c([2H])c5c5c([2H])c([2H])c([2H])c([2H])c54)n3)cc2)c([2H])c1[2H] ; [2H]c1c([2H])c([2H])c2c(c1[2H])c1c([2H])c([2H])c([2H])c([2H])c1n2-c1nc(-c2cccc(-c3ccc(C#N)cc3)c2)nc(-c2ccc3c(c2)oc2ccccc23)n1]

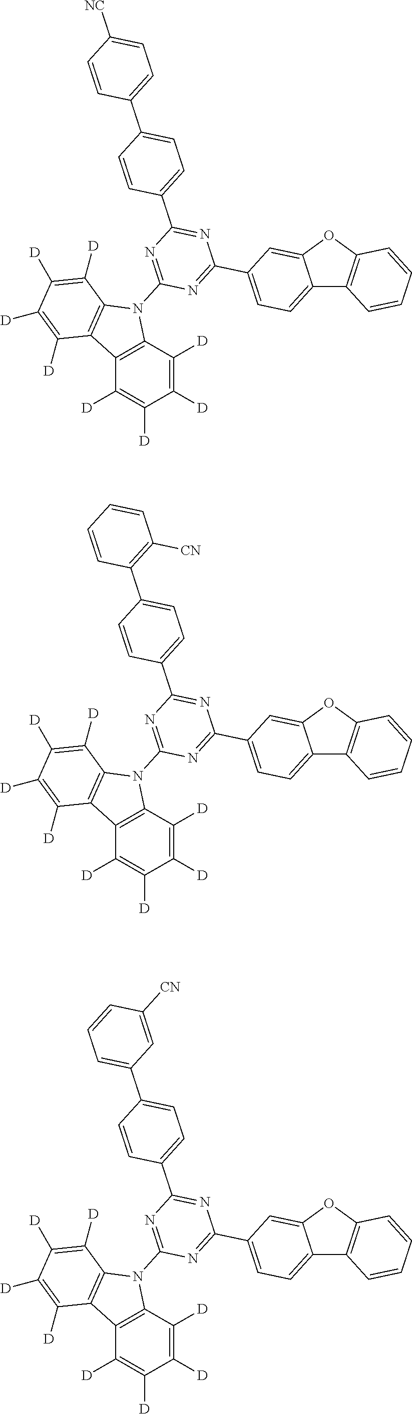 [SMILES: [2H]c1c([2H])c([2H])c2c(c1[2H])c1c([2H])c([2H])c([2H])c([2H])c1n2-c1nc(-c2ccc(-c3ccc(C#N)cc3)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc(-c3ccccc3C#N)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc(-c3cccc(C#N)c3)cc2)nc(-c2ccc3c(c2)oc2ccccc23)n1]

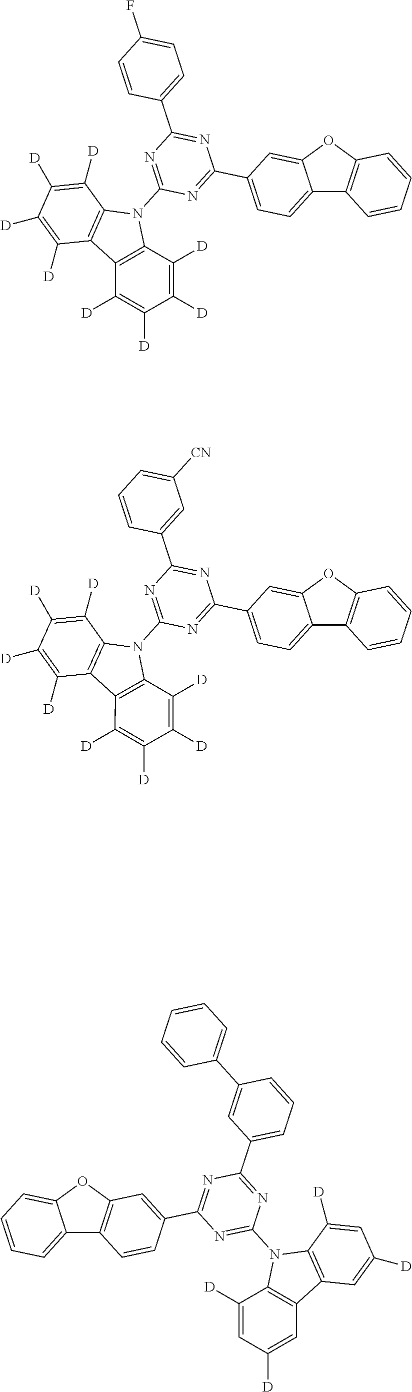 [SMILES: [2H]c1c([2H])c([2H])c2c(c1[2H])c1c([2H])c([2H])c([2H])c([2H])c1n2-c1nc(-c2ccc(F)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2cccc(C#N)c2)nc(-c2ccc3c(c2)oc2ccccc23)n1 ; [2H]c1cc([2H])c2c(c1)c1cc([2H])cc([2H])c1n2-c1nc(-c2cccc(-c3ccccc3)c2)nc(-c2ccc3c(c2)oc2ccccc23)n1]

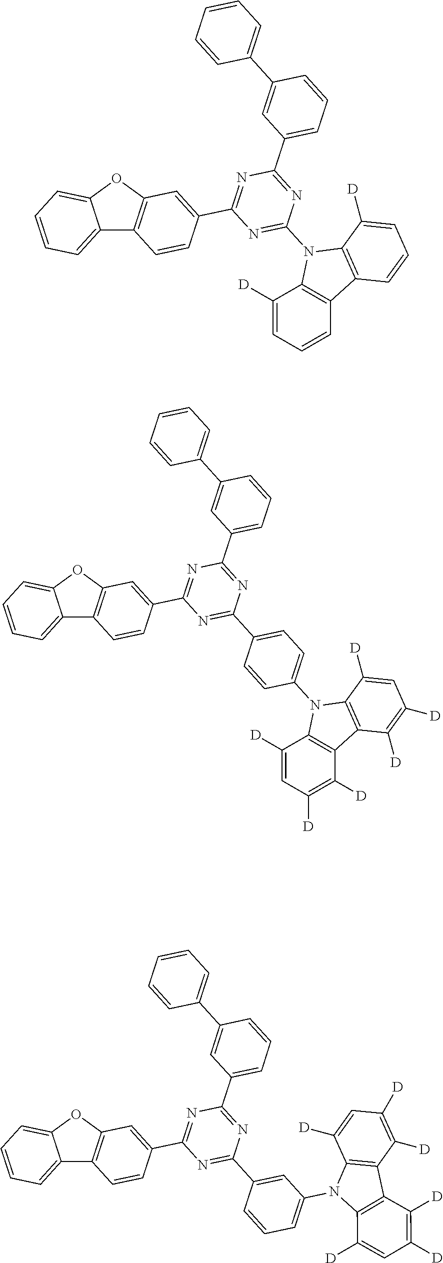 [SMILES: [2H]c1cccc2c3cccc([2H])c3n(-c3nc(-c4cccc(-c5ccccc5)c4)nc(-c4ccc5c(c4)oc4ccccc45)n3)c12 ; [2H]c1cc([2H])c2c(c1[2H])c1c([2H])c([2H])cc([2H])c1n2-c1ccc(-c2nc(-c3cccc(-c4ccccc4)c3)nc(-c3ccc4c(c3)oc3ccccc34)n2)cc1 ; [2H]c1cc([2H])c2c(c1[2H])c1c([2H])c([2H])cc([2H])c1n2-c1cccc(-c2nc(-c3cccc(-c4ccccc4)c3)nc(-c3ccc4c(c3)oc3ccccc34)n2)c1]

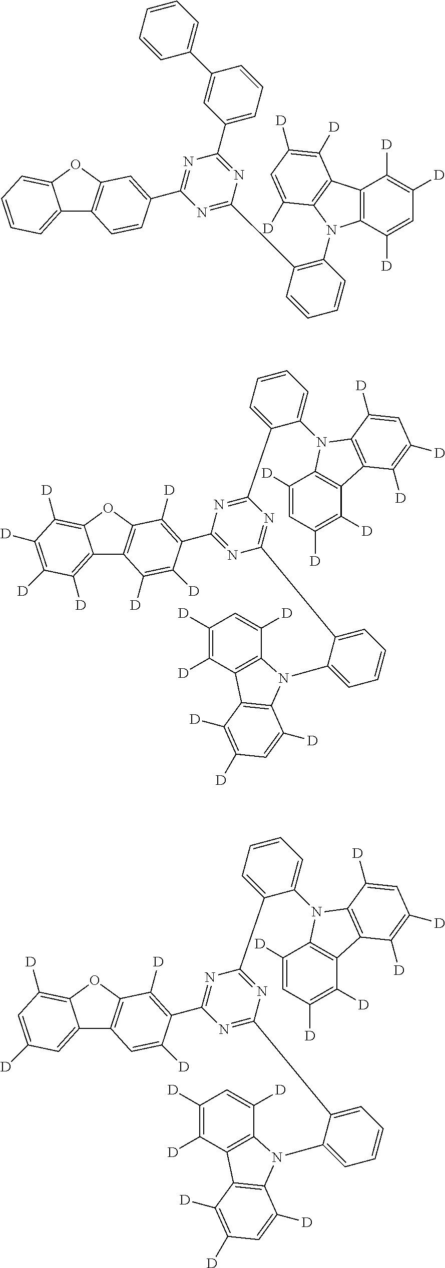

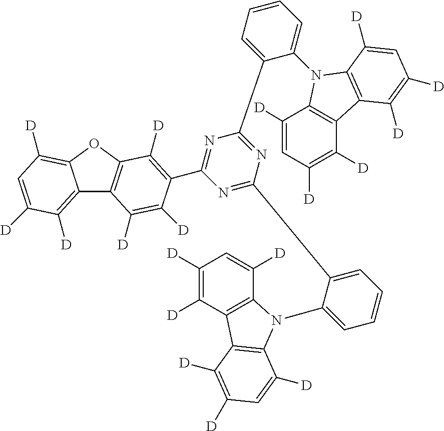 [SMILES: [2H]c1cc([2H])c2oc3c([2H])c(-c4nc(-c5ccccc5-n5c6c([2H])cc([2H])c([2H])c6c6c([2H])c([2H])cc([2H])c65)nc(-c5ccccc5-n5c6c([2H])cc([2H])c([2H])c6c6c([2H])c([2H])cc([2H])c65)n4)c([2H])c([2H])c3c2c1[2H]]

According to yet another embodiment of the present disclosure, there is provided a method for preparing the compound represented by Chemical Formula 1, as shown in the following Reaction Scheme 1.

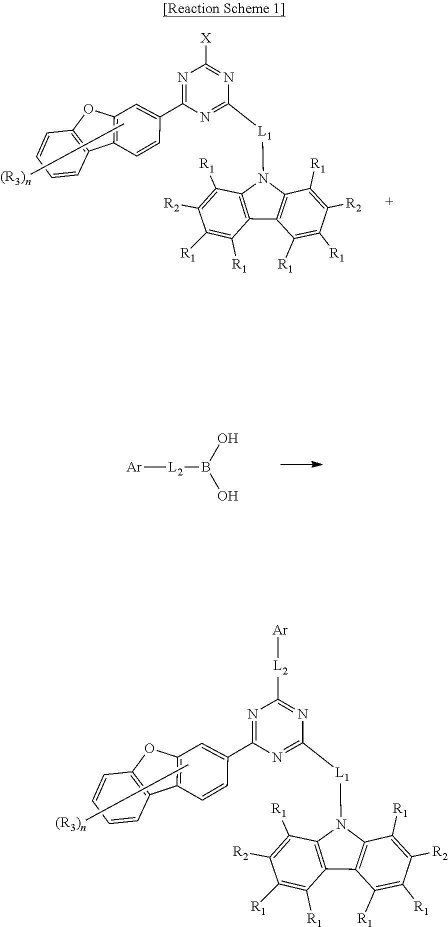

in Reaction Scheme 1, L₁, L₂, Ar, R₁, R₂, R₃, and n are the same as defined in Chemical Formula 1, and X is halogen, preferably chloro or bromo.

Reaction Scheme 1 is a Suzuki coupling reaction, which is preferably carried out in the presence of a palladium catalyst and a base, and a reactive group for the Suzuki coupling reaction can be changed as known in the art. The above preparation method may be further embodied in Preparation Examples described hereinafter.

### Organic Light Emitting Device

In another embodiment of the present disclosure, there is provided an organic light emitting device comprising: a first electrode; a second electrode provided to face the first electrode; and an organic material layer including one or more layers provided between the first electrode and the second electrode, wherein one or more layers of the organic material layer comprise the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, an electron blocking layer, a light emitting layer, an electron transport layer, an electron injection layer and the like as the organic material layer. However, the structure of the organic light emitting device is not limited thereto, and it may include a smaller number of organic layers.

Further, the organic material layer may include a hole injection layer, a hole transport layer, or a layer that simultaneously performs hole injection and transport, wherein the hole injection layer, the hole transport layer, or the layer that simultaneously performs hole injection and transport may include the compound represented by Chemical Formula 1.

Further, the organic material layer may include a light emitting layer, wherein the light emitting layer may include the compound represented by Chemical Formula 1. Particularly, the compound according to the present disclosure may be used as a dopant of the red light emitting layer.

Further, the organic material layer may include a electron transport layer, a electron injection layer, or a layer that simultaneously performs electron transport and electron injection, wherein the electron transport layer, the electron injection layer, or the layer that simultaneously performs electron transport and electron injection may include the compound represented by Chemical Formula 1.

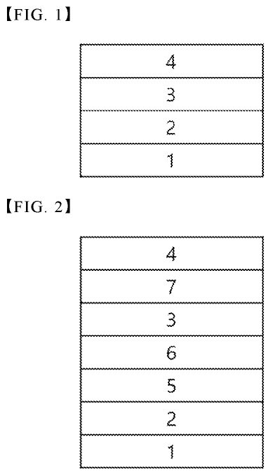

Further, the organic light emitting device according to the present disclosure may be a normal type organic light emitting device in which an anode, one or more organic material layers and a cathode are sequentially stacked on a substrate. Further, the organic light emitting device according to the present disclosure may be an inverted type organic light emitting device in which a cathode, one or more organic material layers and an anode are sequentially stacked on a substrate. For example, the structure of an organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. In such a structure, the compound represented by Chemical Formula 1 may be included in the light emitting layer.

FIG. 2 shows an example of an organic light emitting device comprising a substrate 1, an anode 2, a hole injection layer 5, a hole transport layer 6, a light emitting layer 3, an electron transport layer 7, and a cathode 4. In such a structure, the compound represented by Chemical Formula 1 may be included in the hole transport layer.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers may be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Wherein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzthiazole and benzimidazole-based compound; a poly(p-phenylenevinylene)(PPV)-based polymer; a spiro compound; polyfluorene, lubrene, and the like, but are not limited thereto.

The light emitting layer may include a host material and a dopant material. The host material may be a fused aromatic ring derivative, a heterocycle-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound of an arylamine, which is unsubstituted or substituted with one or two or more substituent groups selected from the group consisting of an aryl group, a silyl group, an alkyl group, a cycloalkyl group, and an arylamino group, and which is substituted with at least one arylvinyl group. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material is suitably a material which may receive electrons well from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure may be a bottom emission device, a top emission device, or a double-sided light emitting device.

In addition, the compound according to the present disclosure may be included in an organic solar cell or an organic transistor in addition to an organic light emitting device.

Hereinafter, the preparation of the compound represented by Chemical Formula 1 and the organic light emitting device including the same will be specifically described in the following Examples. However, the following Examples are provided for illustrative purposes only, and are not intended to limit the scope of the present disclosure.

### PREPARATION EXAMPLE

### Preparation Example 1-1: Preparation of Intermediate Compound Sub 1A

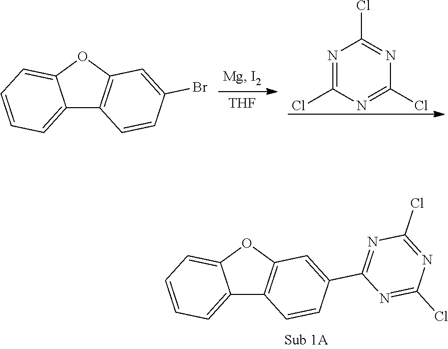 [SMILES: Brc1ccc2c(c1)oc1ccccc12 ; Clc1nc(Cl)nc(Cl)n1 ; Clc1nc(Cl)nc(-c2ccc3c(c2)oc2ccccc23)n1 ; "THF"]

Magnesium (5.4 g, 224 mmol) and iodine (1.0 g, 4.07 mmol) were added to 200 ml of tetrahydrofuran (THF) under a nitrogen environment, and then the mixture was stirred for 30 minutes, and 3-bromodibenzo[b,d]furan (50 g, 203 mmol) dissolved in 200 L of THF was slowly added dropwise thereto at 0° C. for 30 minutes.

The reaction solution was slowly dropwise added to a solution of THF/cyanuric chloride (37.2 g, 203 mmol) at 0° C. for 30 minutes. After completion of the reaction, water was added to the reaction solution, the mixture was extracted with chloroform, then anhydrous magnesium sulfate was added, stirred, filtered, and the filtrate was distilled under reduced pressure. The concentrated compound was purified by a column chromatography to prepare Intermediate Compound Sub 1A (40.3 g, 63%, MS: [M+H]+=316.0).

### Preparation Example 1-2: Preparation of Intermediate Compound Sub 1B

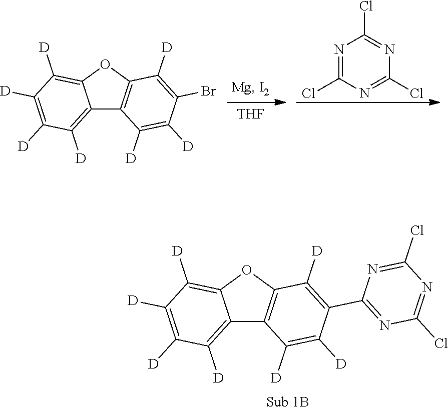 [SMILES: [2H]c1c([2H])c([2H])c2c(oc3c([2H])c(Br)c([2H])c([2H])c32)c1[2H] ; Clc1nc(Cl)nc(Cl)n1 ; [2H]c1c([2H])c([2H])c2c(oc3c([2H])c(-c4nc(Cl)nc(Cl)n4)c([2H])c([2H])c32)c1[2H] ; "THF"]

Magnesium (5.2 g, 217.4 mmol) and iodine (1.0 g, 3.95 mmol) were added to 200 ml of tetrahydrofuran (THF) under a nitrogen environment, and the mixture was stirred for 30 minutes, and then 3-bromodibenzo[b,d]furan-1,2,4,6,7,8,9-d7 (50 g, 197.6 mmol) dissolved in 200 L of THF was slowly added dropwise thereto at 0° C. for 30 minutes. The reaction solution was slowly dropwise added to a solution of THF/cyanuric chloride (36.1 g, 197.6 mmol) at 0° C. for 30 minutes. After completion of the reaction, water was added to the reaction solution, the mixture was extracted with chloroform, then anhydrous magnesium sulfate was added, stirred, filtered, and the filtrate was distilled under reduced pressure. The concentrated compound was purified by a column chromatography to prepare Intermediate Compound Sub 1B (36.7 g, 59%, MS: [M+H]+=323.0).

### Preparation Example 1-3: Preparation of Intermediate Compound Sub 1C

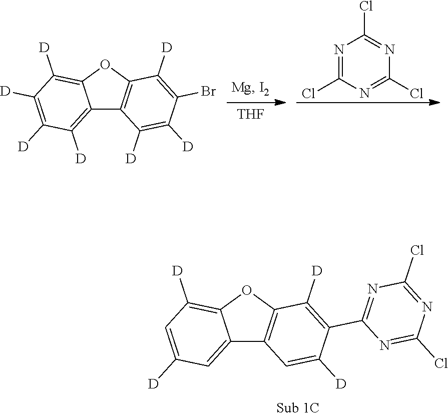 [SMILES: [2H]c1c([2H])c([2H])c2c(oc3c([2H])c(Br)c([2H])c([2H])c32)c1[2H] ; Clc1nc(Cl)nc(Cl)n1 ; [2H]c1cc([2H])c2oc3c([2H])c(-c4nc(Cl)nc(Cl)n4)c([2H])cc3c2c1 ; "THF"]

Magnesium (5.4 g, 224.5 mmol) and iodine(1.0 g, 4.08 mmol) were added to 200 ml of tetrahydrofuran (THF) under a nitrogen environment, and the mixture was stirred for 30 minutes, and then 3-bromodibenzo[b,d]furan-2,4,6,8-d4 (50 g, 204.1 mmol) dissolved in 200 L of THF was slowly added dropwise thereto at 0° C. for 30 minutes. The reaction solution was slowly dropwise added to a solution of THF/cyanuric chloride (37.3 g, 204.1 mmol) at 0° C. for 30 minutes. After completion of the reaction, water was added to the reaction solution, the mixture was extracted with chloroform, then anhydrous magnesium sulfate was added, stirred, filtered, and the filtrate was distilled under reduced pressure. The concentrated compound was purified by a column chromatography to prepare Intermediate Compound Sub 1C (38.6 g, 60%, MS: [M+H]+=320.0).

### Preparation Example 1-4: Preparation of Intermediate Compound Sub 1D

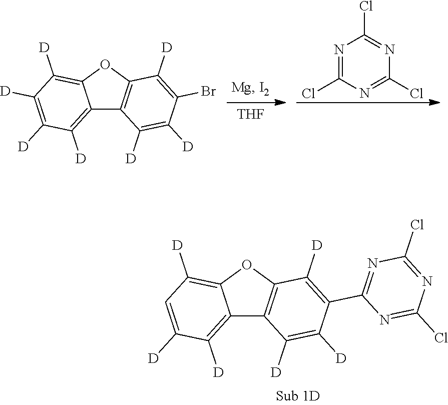 [SMILES: [2H]c1c([2H])c([2H])c2c(oc3c([2H])c(Br)c([2H])c([2H])c32)c1[2H] ; Clc1nc(Cl)nc(Cl)n1 ; [2H]c1cc([2H])c2oc3c([2H])c(-c4nc(Cl)nc(Cl)n4)c([2H])c([2H])c3c2c1[2H] ; "THF"]

Magnesium (5.1 g, 217.6 mmol) and iodine (1.0 g, 3.99 mmol) were added to 200 ml of tetrahydrofuran (THF) under a nitrogen environment, and the mixture was stirred for 30 minutes, and then 3-bromodibenzo[b,d]furan-1,2,4,6,8,9-d6 (50 g, 197.9 mmol) dissolved in 200 L of THF was slowly added dropwise thereto at 0° C. for 30 minutes. The reaction solution was slowly dropwise added to a solution of THF/cyanuric chloride (36.1 g, 197.9 mmol) at 0° C. for 30 minutes. After completion of the reaction, water was added to the reaction solution, the mixture was extracted with chloroform, then anhydrous magnesium sulfate was added, stirred, filtered, and the filtrate was distilled under reduced pressure. The concentrated compound was purified by a column chromatography to prepare Intermediate Compound Sub 1D (39.8 g, 65%, MS: [M+H]+=322.0).

### Preparation Example 1-5: Preparation of Intermediate Compound Sub 2-1

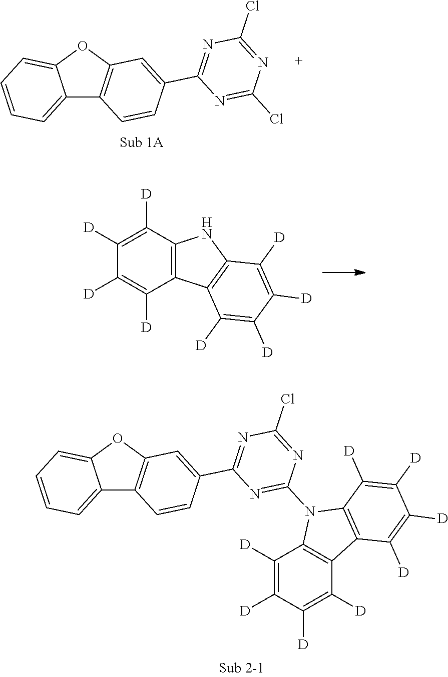 [SMILES: Clc1nc(Cl)nc(-c2ccc3c(c2)oc2ccccc23)n1 ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1nc(Cl)nc(-c2ccc3c(c2)oc2ccccc23)n1]

Sub 1A (10 g, 31.7 mmol) and 9H-carbazole-1,2,3,4,5,6,7,8-d8 (5.6 g, 31.7 mmol) were added to 200 ml of xylene under a nitrogen atmosphere, and the mixture was stirred and refluxed. Then, sodium tert-butoxide (9.2 g, 95.2 mmol) was added thereto, and the mixture was sufficiently stirred, and then bis(tri-tert-butylphosphine)palladium (0.5 g, 1 mmol) was added. After the reaction for 4 hours, the reaction mixture was cooled to room temperature, and the resulting solid was filtered. The solid was dissolved in 433 mL of chloroform, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare a white solid compound Sub 2-1 (9.8 g, 68%, MS: [M+H]+=455.1).

### Preparation Example 1-6: Preparation of Intermediate Compound Sub 2-2

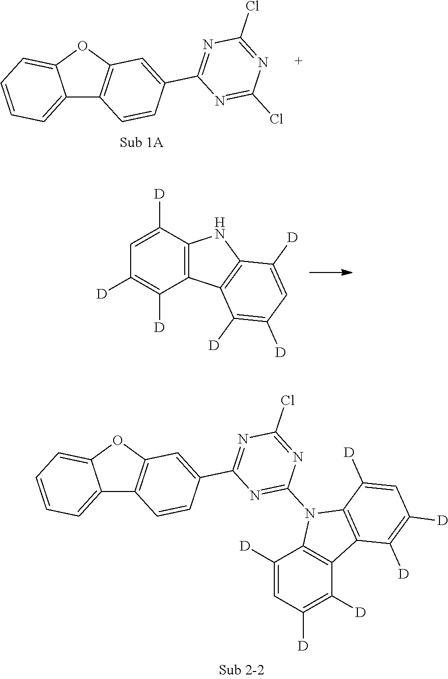 [SMILES: Clc1nc(Cl)nc(-c2ccc3c(c2)oc2ccccc23)n1 ; [2H]c1cc([2H])c2[nH]c3c([2H])cc([2H])c([2H])c3c2c1[2H] ; [2H]c1cc([2H])c2c(c1[2H])c1c([2H])c([2H])cc([2H])c1n2-c1nc(Cl)nc(-c2ccc3c(c2)oc2ccccc23)n1]

Sub 1A (50 g, 83.7 mmol) and 9H-carbazole-1,3,4,5,6,8-d6 (14.5 g, 83.7 mmol) were added to 1000 ml of xylene under a nitrogen atmosphere, and the mixture was stirred and refluxed. Then, sodium tert-butoxide (24.1 g, 251.2 mmol) was added thereto, and the mixture was sufficiently stirred, and then bis(tri-tert-butylphosphine)palladium (1.3 g, 2.5 mmol) was added. After the reaction for 3 hours, the reaction mixture was cooled to room temperature, and the resulting solid was filtered. The solid was dissolved in 1136 mL of chloroform, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare a white solid compound Sub 2-2 (23.8 g, 63%, MS: [M+H]+=453.1).

### Preparation Example 1-7: Preparation of Intermediate Compound Sub 2-3

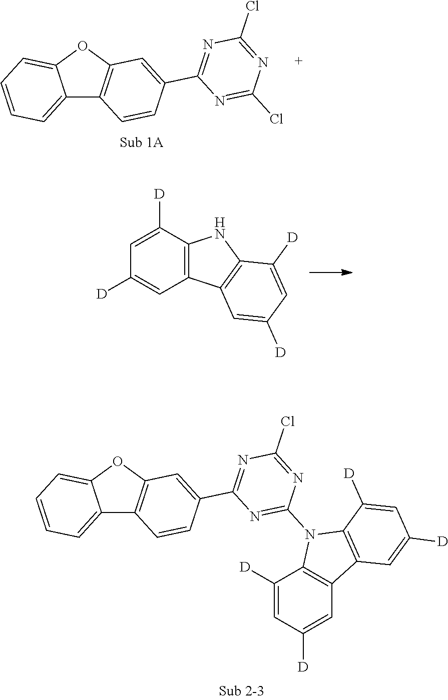 [SMILES: Clc1nc(Cl)nc(-c2ccc3c(c2)oc2ccccc23)n1 ; [2H]c1cc([2H])c2[nH]c3c([2H])cc([2H])cc3c2c1 ; [2H]c1cc([2H])c2c(c1)c1cc([2H])cc([2H])c1n2-c1nc(Cl)nc(-c2ccc3c(c2)oc2ccccc23)n1]

Sub 1A (50 g, 125 mmol) and 9H-carbazole-1,3,6,8-d4 (21.4 g, 125 mmol) were added to 1000 ml of xylene under a nitrogen atmosphere, and the mixture was stirred and refluxed. Then, sodium tert-butoxide (36 g, 375 mmol) was added thereto, and the mixture was sufficiently stirred, and then bis(tri-tert-butylphosphine)palladium (1.9 g, 3.8 mmol) was added. After the reaction for 1 hour, the reaction mixture was cooled to room temperature, and the resulting solid was filtered. The solid was dissolved in 1688 mL of chloroform, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare white solid compound Sub 2-3 (39.4 g, 70%, MS: [M+H]+=451.1).

### Preparation Example 1-8: Preparation of Intermediate Compound Sub 2-4

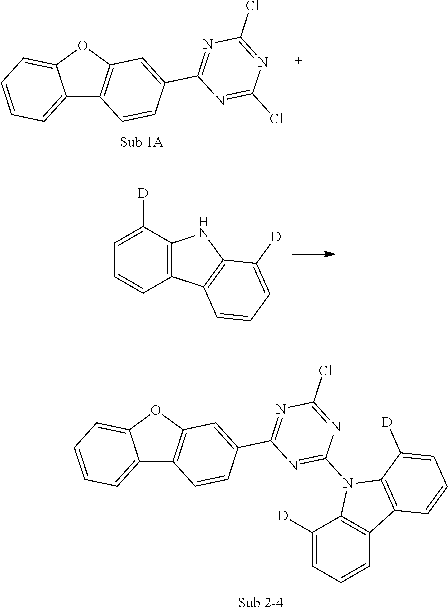 [SMILES: Clc1nc(Cl)nc(-c2ccc3c(c2)oc2ccccc23)n1 ; [2H]c1cccc2c1[nH]c1c([2H])cccc12 ; [2H]c1cccc2c3cccc([2H])c3n(-c3nc(Cl)nc(-c4ccc5c(c4)oc4ccccc45)n3)c12]

Sub 1A (50 g, 100 mmol) and 9H-carbazole-1,8-d2 (16.9 g, 100 mmol) were added to 1000 ml of xylene under a nitrogen atmosphere, and the mixture was stirred and refluxed. Then, sodium tert-butoxide (28.8 g, 300 mmol) was added thereto, and the mixture was sufficiently stirred, and then bis(tri-tert-butylphosphine)palladium (1.5 g, 3 mmol) was added. After the reaction for 4 hours, the reaction mixture was cooled to room temperature, and the resulting solid was filtered. The solid was dissolved in 1344 mL of chloroform, washed twice with water, the organic layer was separated, anhydrous magnesium sulfate was added, stirred, filtered, and the filtrate was distilled under reduced pressure. The concentrated compound was purified through a silica column using chloroform and ethyl acetate to prepare a white solid compound Sub 2-4 (35.4 g, 79%, MS: [M+H]+=449.1).

### Preparation Example 1-9: Preparation of Intermediate Compound Sub 2-5

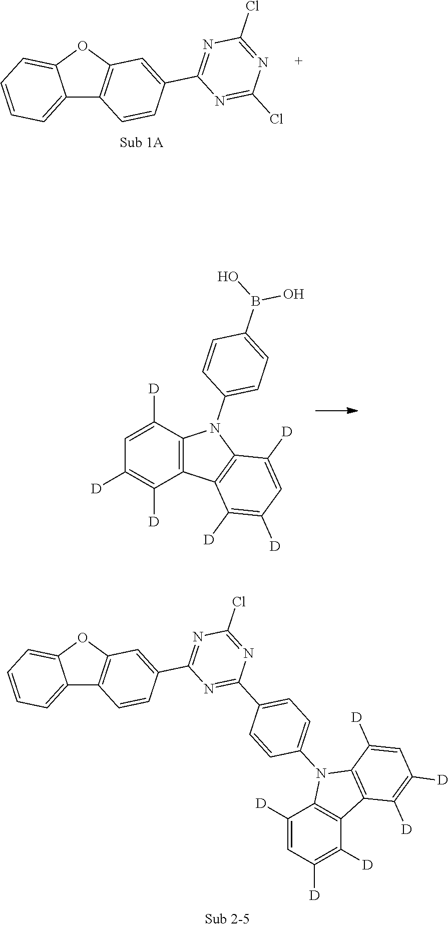 [SMILES: Clc1nc(Cl)nc(-c2ccc3c(c2)oc2ccccc23)n1 ; [2H]c1cc([2H])c2c(c1[2H])c1c([2H])c([2H])cc([2H])c1n2-c1ccc(B(O)O)cc1 ; [2H]c1cc([2H])c2c(c1[2H])c1c([2H])c([2H])cc([2H])c1n2-c1ccc(-c2nc(Cl)nc(-c3ccc4c(c3)oc3ccccc34)n2)cc1]

Sub 1A (30 g, 95.2 mmol) and (4-(9H-carbazol-9-yl-1,3,4,5,6,8-d6)phenyl)boronic acid (58.6 g, 200 mmol) were added to 900 ml of tetrahydrofuran under a nitrogen atmosphere, and the mixture was stirred and refluxed. Then, potassium carbonate (39.5 g, 285.7 mmol) was dissolved in 39 ml of water and added thereto, and the mixture was sufficiently stirred, and then tetrakistriphenyl-phosphinopalladium (4.4 g, 3.8 mmol) was added. After the reaction for 3 hours, the reaction mixture was cooled to room temperature, and then the organic layer and the aqueous layer were separated, and the organic layer was distilled. This was again added to 1006 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound Sub 2-5 (34.7 g, 69%, MS: [M+H]+=529.2).

### Preparation Example 1-10: Preparation of Intermediate Compound Sub 2-6

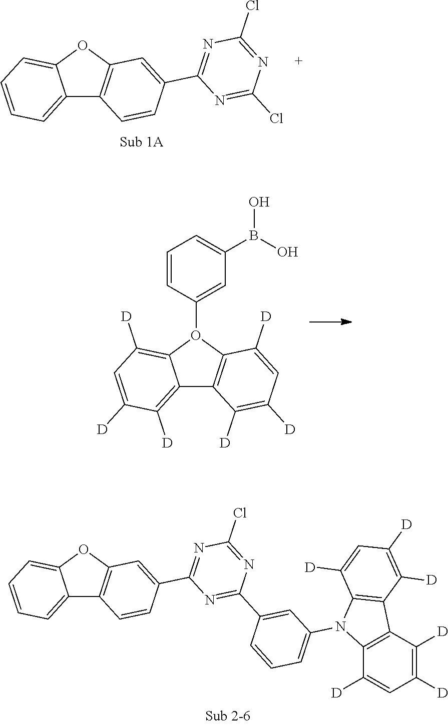

Sub 1A (30 g, 95.2 mmol) and (3-(9H-carbazol-9-yl-1,3,4,5,6,8-d6)phenyl)boronic acid (58.6 g, 200 mmol) were added to 900 ml of tetrahydrofuran under a nitrogen atmosphere, and the mixture was stirred and refluxed. Then, potassium carbonate (39.5 g, 285.7 mmol) was dissolved in 39 ml of water and added thereto, and the mixture was sufficiently stirred, and then tetrakistriphenyl-phosphinopalladium (4.4 g, 3.8 mmol) was added. After the reaction for 2 hours, the reaction mixture was cooled to room temperature, and then the organic layer and the aqueous layer were separated, and the organic layer was distilled. This was again added to 1006 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound Sub 2-6(30.2 g, 60%, MS: [M+H]+=529.2).

### Preparation Example 1-11: Preparation of Intermediate Compound Sub 2-7

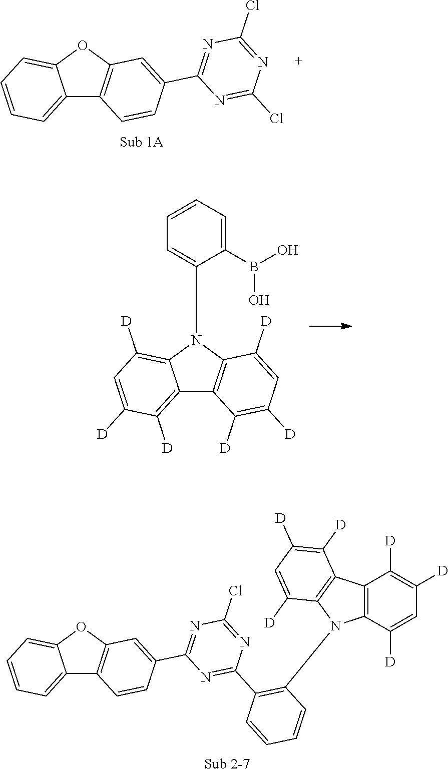 [SMILES: Clc1nc(Cl)nc(-c2ccc3c(c2)oc2ccccc23)n1 ; [2H]c1cc([2H])c2c(c1[2H])c1c([2H])c([2H])cc([2H])c1n2-c1ccccc1B(O)O ; [2H]c1cc([2H])c2c(c1[2H])c1c([2H])c([2H])cc([2H])c1n2-c1ccccc1-c1nc(Cl)nc(-c2ccc3c(c2)oc2ccccc23)n1]

Sub 1A (30 g, 95.2 mmol) and (2-(9H-carbazol-9-yl-1,3,4,5,6,8-d6)phenyl)boronic acid (58.6 g, 200 mmol) were added to 900 ml of tetrahydrofuran under a nitrogen atmosphere, and the mixture was stirred and refluxed. Then, potassium carbonate (39.5 g, 285.7 mmol) was dissolved in 39 ml of water and added thereto, and the mixture was sufficiently stirred, and then tetrakistriphenyl-phosphinopalladium (4.4 g, 3.8 mmol) was added. After the reaction for 1 hour, the reaction mixture was cooled to room temperature, and then the organic layer and the aqueous layer were separated, and the organic layer was distilled. This was again added to 1006 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound Sub 2-7 (25.7 g, 51%, MS: [M+H]+=529.2).

### EXAMPLE

### Example 1: Preparation of Compound 1

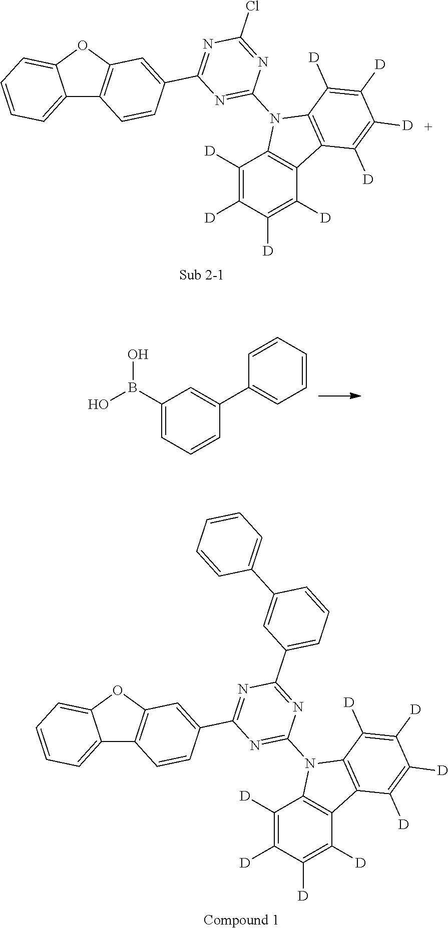 [SMILES: [2H]c1c([2H])c([2H])c2c(c1[2H])c1c([2H])c([2H])c([2H])c([2H])c1n2-c1nc(Cl)nc(-c2ccc3c(c2)oc2ccccc23)n1 ; OB(O)c1cccc(-c2ccccc2)c1 ; [2H]c1c([2H])c([2H])c2c(c1[2H])c1c([2H])c([2H])c([2H])c([2H])c1n2-c1nc(-c2cccc(-c3ccccc3)c2)nc(-c2ccc3c(c2)oc2ccccc23)n1]

Sub 2-1 (20 g, 44 mmol) and [1,1′-biphenyl]-3-ylboronic acid (8.7 g, 44 mmol) were added to 400 ml of tetrahydrofuran under a nitrogen atmosphere, and the mixture was stirred and refluxed. Then, potassium carbonate (18.3 g, 132.1 mmol) was dissolved in 18 ml of water and added thereto, and the mixture was sufficiently stirred, and then tetrakistriphenyl-phosphinopalladium (1.5 g, 1.3 mmol) was added. After the reaction for 1 hour, the reaction mixture was cooled to room temperature, and then the organic layer and the aqueous layer were separated, and the organic layer was distilled. This was again added to 507 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 1 (20.3 g, 80%, MS: [M+H]+=576.3).

### Example 2: Preparation of Compound 2

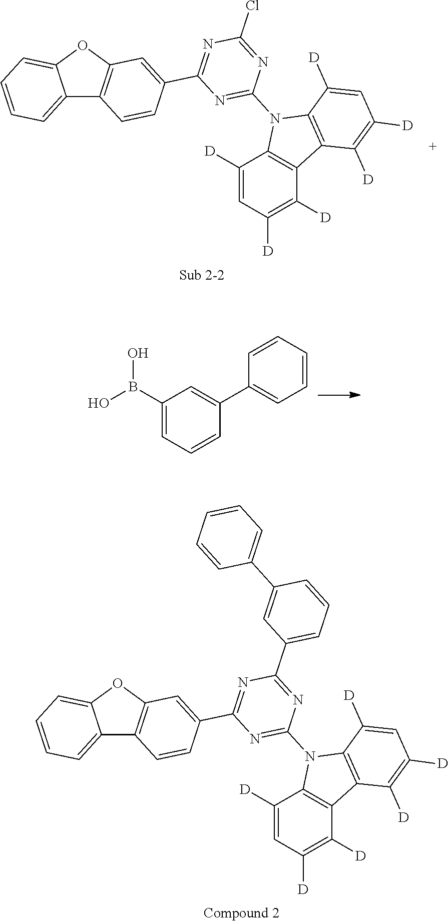 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1cccc(-c2ccccc2)c1 ; [2H]c1cc([2H])c2c(c1[2H])c1c([2H])c([2H])cc([2H])c1n2-c1nc(-c2cccc(-c3ccccc3)c2)nc(-c2ccc3c(c2)oc2ccccc23)n1]

Sub 2-2 (20 g, 44.2 mmol) and [1,1′-biphenyl]-3-ylboronic acid (8.8 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 507 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 2 (18.5 g, 73%, MS: [M+H]+=574.3).

### Example 3: Preparation of Compound 3

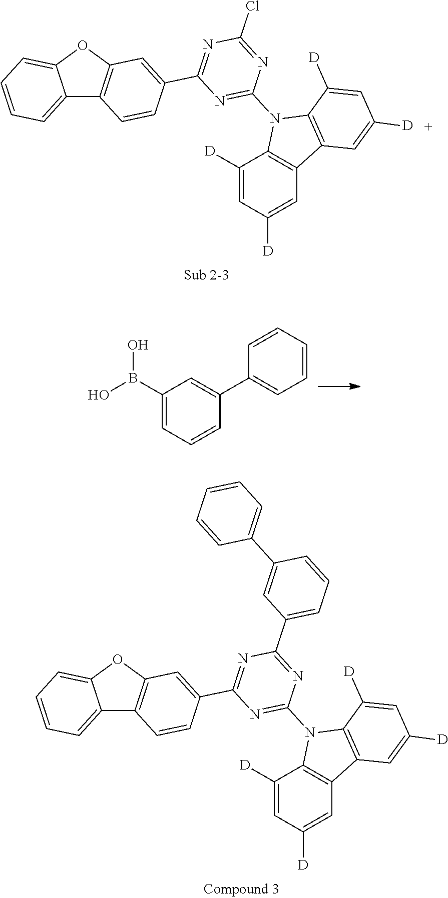 [SMILES: [2H]c1cc([2H])c2c(c1)c1cc([2H])cc([2H])c1n2-c1nc(Cl)nc(-c2ccc3c(c2)oc2ccccc23)n1 ; OB(O)c1cccc(-c2ccccc2)c1 ; [2H]c1cc([2H])c2c(c1)c1cc([2H])cc([2H])c1n2-c1nc(-c2cccc(-c3ccccc3)c2)nc(-c2ccc3c(c2)oc2ccccc23)n1]

Sub 2-3 (20 g, 44.4 mmol) and [1,1′-biphenyl]-3-ylboronic acid (8.8 g, 44.4 mmol) were added to 400 ml of tetrahydrofuran under a nitrogen atmosphere, and the mixture was stirred and refluxed. Then, potassium carbonate (18.4 g, 133.3 mmol) was dissolved in 18 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 508 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 3 (14.5 g, 57%, MS: [M+H]+=572.3).

### Example 4: Preparation of Compound 4

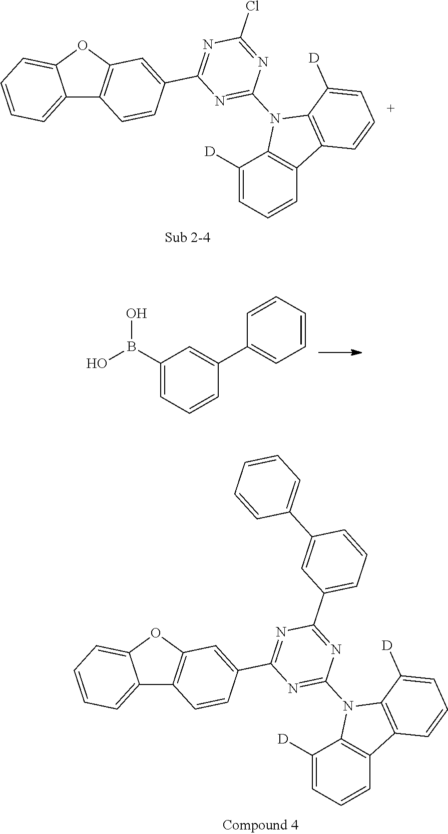

Sub 2-4 (20 g, 44.6 mmol) and [1,1′-biphenyl]-3-ylboronic acid (8.8 g, 44.6 mmol) were added to 400 ml of tetrahydrofuran under a nitrogen atmosphere, and the mixture was stirred and refluxed. Then, potassium carbonate (18.5 g, 133.9 mmol) was dissolved in 19 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 508 mL of chloroform, dissolved and washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 4 (17.3 g, 68%, MS: [M+H]+=570.3).

### Example 5: Preparation of Compound 5

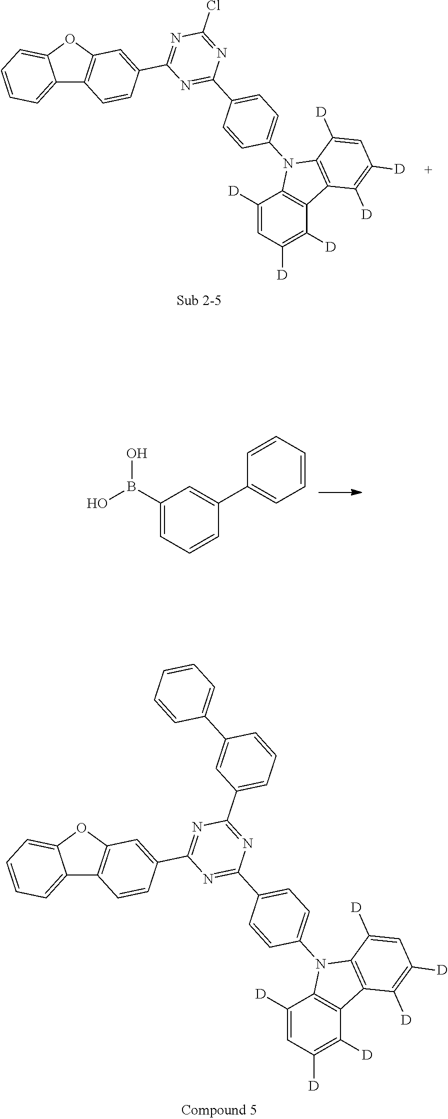

Sub 2-5 (20 g, 37.9 mmol) and [1,1′-biphenyl]-3-ylboronic acid (7.5 g, 37.9 mmol) were added to 400 ml of tetrahydrofuran under a nitrogen atmosphere, and the mixture was stirred and refluxed. Then, potassium carbonate (15.7 g, 113.6 mmol) was dissolved in 16 ml of water and added thereto, and the mixture was sufficiently stirred, and then tetrakistriphenyl-phosphinopalladium (1.3 g, 1.1 mmol) was added. After the reaction for 2 hours, the reaction mixture was cooled to room temperature, and then the organic layer and the aqueous layer were separated, and the organic layer was distilled. This was again added to 489 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 5 (15.7 g, 64%, MS: [M+H]+=647.3).

### Example 6: Preparation of Compound 6

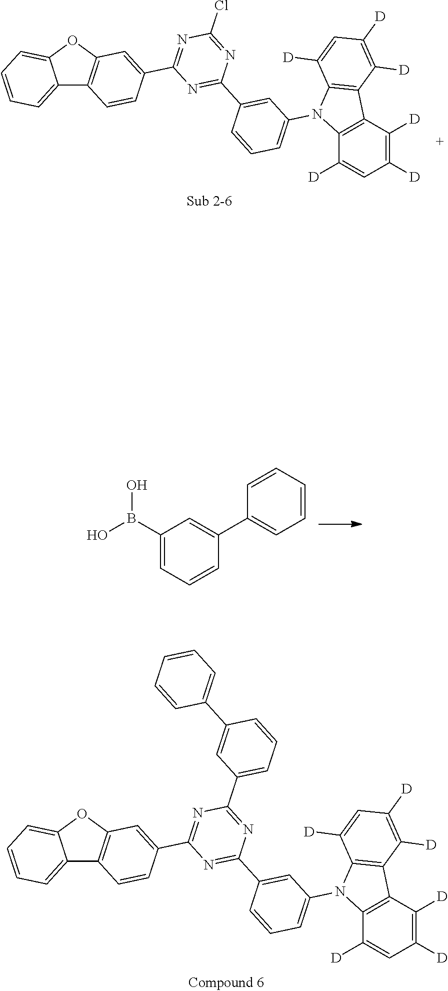

Sub 2-6 (20 g, 37.9 mmol) and [1,1′-biphenyl]-3-ylboronic acid (7.5 g, 37.9 mmol) were added to 400 ml of tetrahydrofuran under a nitrogen atmosphere, and the mixture was stirred and refluxed. Then, potassium carbonate (15.7 g, 113.6 mmol) was dissolved in 16 ml of water and added thereto, and the mixture was sufficiently stirred, and then tetrakistriphenyl-phosphinopalladium (1.3 g, 1.1 mmol) was added. After the reaction for 3 hours, the reaction mixture was cooled to room temperature, and then the organic layer and the aqueous layer were separated, and the organic layer was distilled. This was again added to 489 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 6 (12.2 g, 50%, MS: [M+H]+=647.3).

### Example 7: Preparation of Compound 7

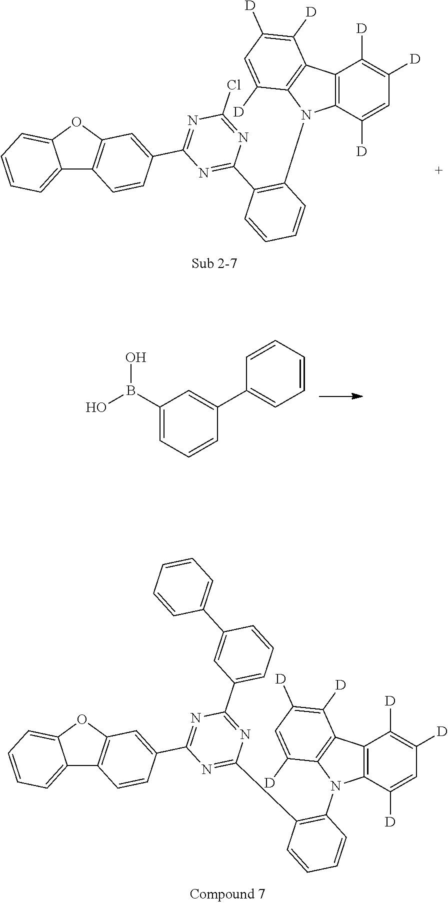

Sub 2-7 (20 g, 37.9 mmol) and [1,1′-biphenyl]-3-ylboronic acid (7.5 g, 37.9 mmol) were added to 400 ml of tetrahydrofuran under a nitrogen atmosphere, and the mixture was stirred and refluxed. Then, potassium carbonate (15.7 g, 113.6 mmol) was dissolved in 16 ml of water and added thereto, and the mixture was sufficiently stirred, and then tetrakistriphenyl-phosphinopalladium (1.3 g, 1.1 mmol) was added. After the reaction for 1 hour, the reaction mixture was cooled to room temperature, and then the organic layer and the aqueous layer were separated, and the organic layer was distilled. This was again added to 489 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 7 (13.5 g, 55%, MS: [M+H]+=647.3).

### Example 8: Preparation of Compound 8

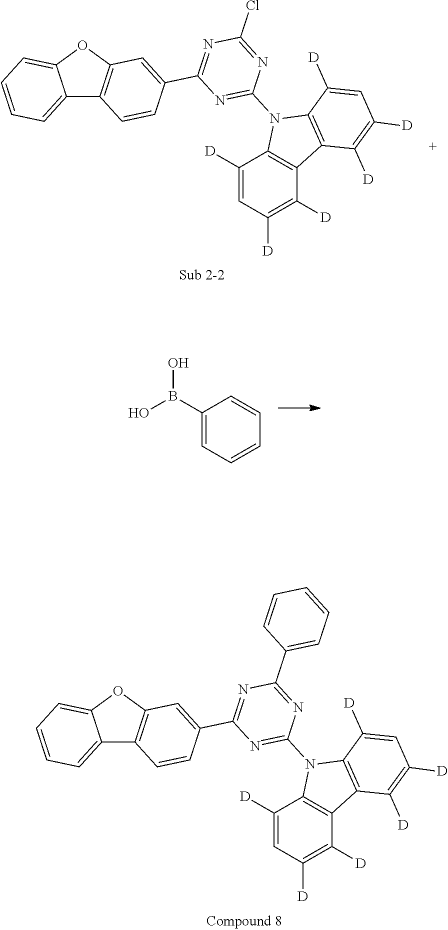 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1ccccc1 ; [2H]c1cc([2H])c2c(c1[2H])c1c([2H])c([2H])cc([2H])c1n2-c1nc(-c2ccccc2)nc(-c2ccc3c(c2)oc2ccccc23)n1]

Sub 2-2 (20 g, 44.2 mmol) and phenylboronic acid (5.4 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 437 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 8 (12.9 g, 59%, MS: [M+H]+=495.2).

### Example 9: Preparation of Compound 9

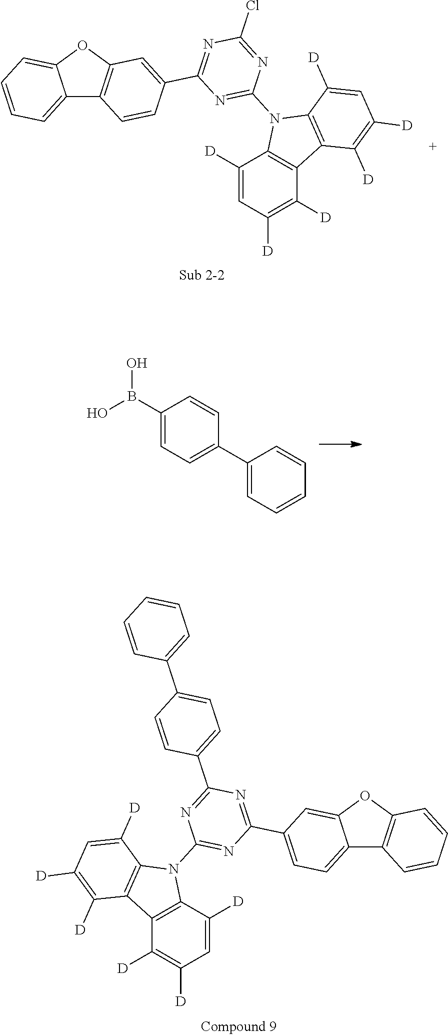 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1ccc(-c2ccccc2)cc1 ; [2H]c1cc([2H])c2c(c1[2H])c1c([2H])c([2H])cc([2H])c1n2-c1nc(-c2ccc(-c3ccccc3)cc2)nc(-c2ccc3c(c2)oc2ccccc23)n1]

Sub 2-2 (20 g, 44.2 mmol) and [1,1′-biphenyl]-4-ylboronic acid (8.8 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 1 hour, the reaction mixture was cooled to room temperature, and then the organic layer and the aqueous layer were separated, and the organic layer was distilled. This was again added to 504 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 9 (17.9 g, 71%, MS: [M+H]+=571.2).

### Example 10: Preparation of Compound 10

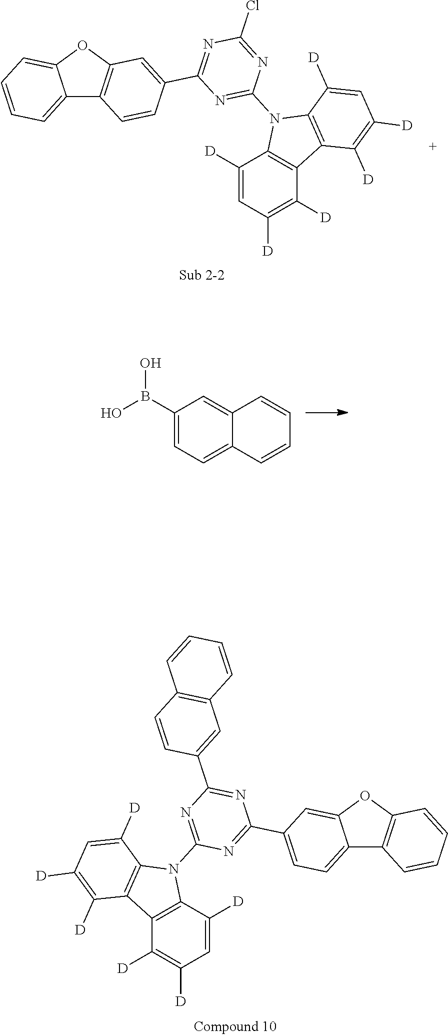 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1ccc2ccccc2c1 ; [2H]c1cc([2H])c2c(c1[2H])c1c([2H])c([2H])cc([2H])c1n2-c1nc(-c2ccc3ccccc3c2)nc(-c2ccc3c(c2)oc2ccccc23)n1]

Sub 2-2 (20 g, 44.2 mmol) and naphthalen-2-ylboronic acid (7.6 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 3 hours, the reaction mixture was cooled to room temperature, and then the organic layer and the aqueous layer were separated, and the organic layer was distilled. This was again added to 481 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 10 (16.6 g, 69%, MS: [M+H]+=545.2).

### Example 11: Preparation of Compound 11

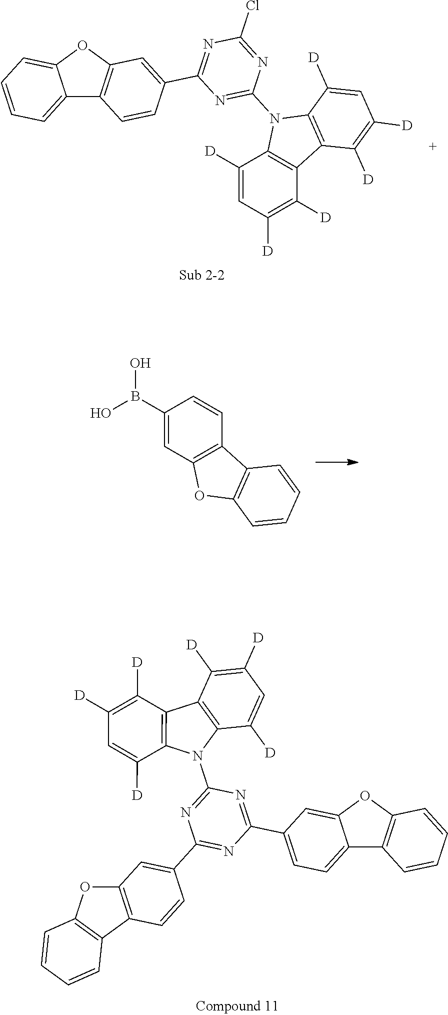 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1ccc2c(c1)oc1ccccc12 ; [2H]c1cc([2H])c2c(c1[2H])c1c([2H])c([2H])cc([2H])c1n2-c1nc(-c2ccc3c(c2)oc2ccccc23)nc(-c2ccc3c(c2)oc2ccccc23)n1]

Sub 2-2 (20 g, 44.2 mmol) and dibenzo[b,d]furan-3-ylboronic acid (9.4 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 517 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 11 (19.6 g, 76%, MS: [M+H]+=585.2).

### Example 12: Preparation of Compound 12

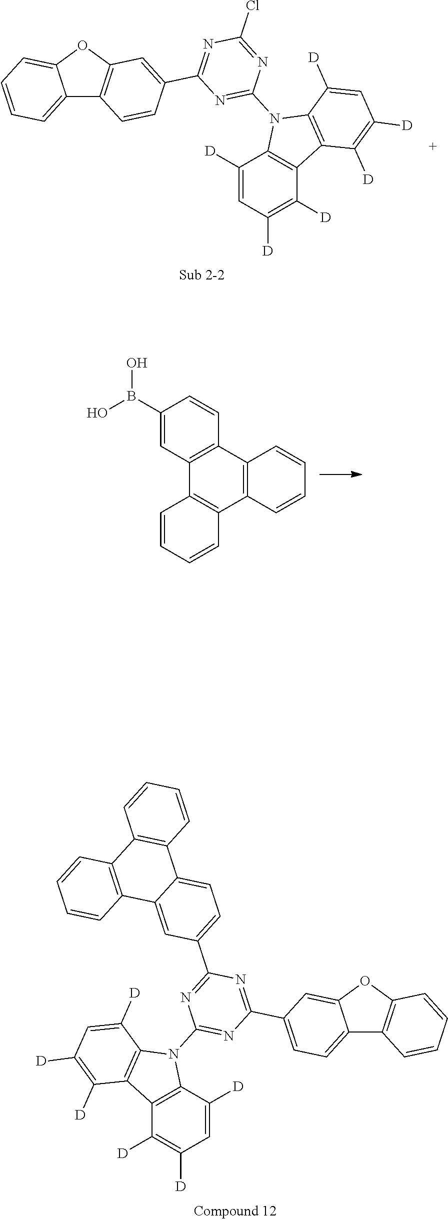 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1ccc2c3ccccc3c3ccccc3c2c1 ; [2H]c1cc([2H])c2c(c1[2H])c1c([2H])c([2H])cc([2H])c1n2-c1nc(-c2ccc3c(c2)oc2ccccc23)nc(-c2ccc3c4ccccc4c4ccccc4c3c2)n1]

Sub 2-2 (20 g, 44.2 mmol) and triphenylen-2-ylboronic acid (12 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 570 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 12 (21.7 g, 76%, MS: [M+H]+=645.3).

### Example 13: Preparation of Compound 13

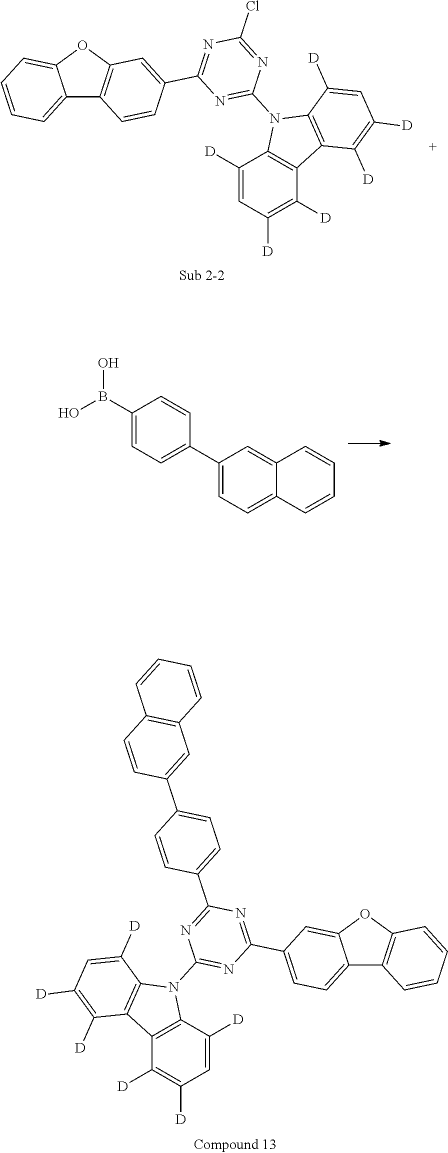 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; OB(O)c1ccc(-c2ccc3ccccc3c2)cc1 ; [2H]c1cc([2H])c2c(c1[2H])c1c([2H])c([2H])cc([2H])c1n2-c1nc(-c2ccc(-c3ccc4ccccc4c3)cc2)nc(-c2ccc3c(c2)oc2ccccc23)n1]

Sub 2-2 (20 g, 44.2 mmol) and (4-(naphthalen-2-yl)phenyl)boronic acid (5.4 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 2 hours, the reaction mixture was cooled to room temperature, and then the organic layer and the aqueous layer were separated, and the organic layer was distilled. This was again added to 549 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 13 (15.6 g, 57%, MS: [M+H]+=621.3).

### Example 14: Preparation of Compound 14

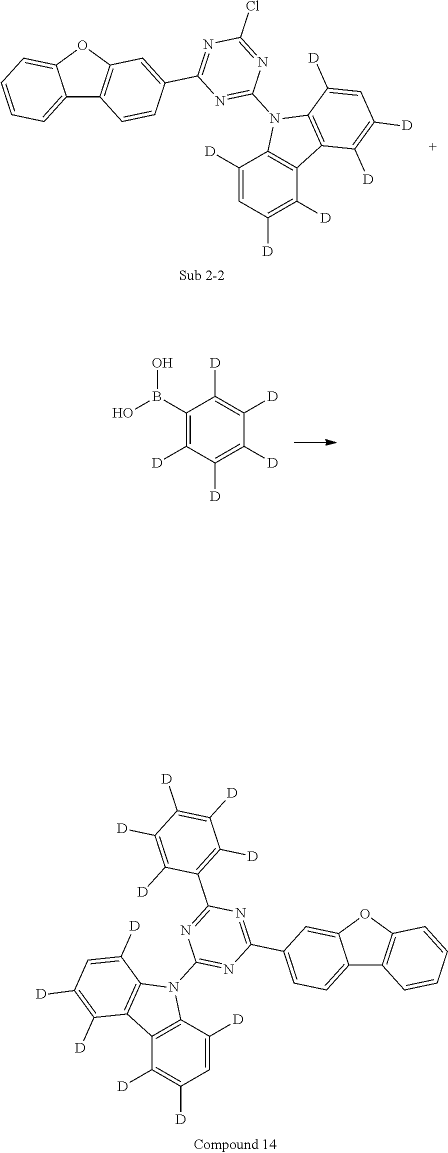 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; [2H]c1c([2H])c([2H])c(B(O)O)c([2H])c1[2H] ; [2H]c1cc([2H])c2c(c1[2H])c1c([2H])c([2H])cc([2H])c1n2-c1nc(-c2ccc3c(c2)oc2ccccc23)nc(-c2c([2H])c([2H])c([2H])c([2H])c2[2H])n1]

Sub 2-2 (20 g, 44.2 mmol) and (phenyl-d5)boronic acid (5.6 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 3 hours, the reaction mixture was cooled to room temperature, and then the organic layer and the aqueous layer were separated, and the organic layer was distilled. This was again added to 504 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 14 (13.4 g, 53%, MS: [M+H]+=571.2).

### Example 15: Preparation of Compound 15

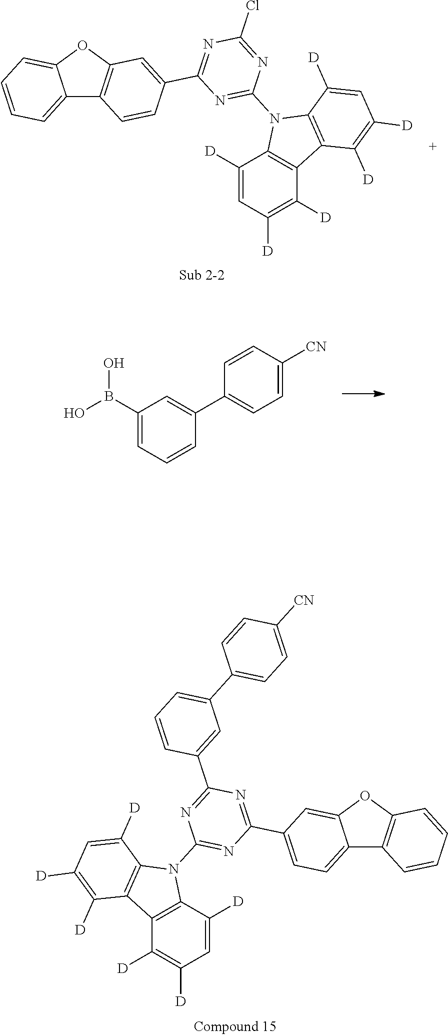 [SMILES: [2H]c1cc([2H])c2c(c1[2H])c1c([2H])c([2H])cc([2H])c1n2-c1nc(Cl)nc(-c2ccc3c(c2)oc2ccccc23)n1 ; N#Cc1ccc(-c2cccc(B(O)O)c2)cc1 ; [2H]c1cc([2H])c2c(c1[2H])c1c([2H])c([2H])cc([2H])c1n2-c1nc(-c2cccc(-c3ccc(C#N)cc3)c2)nc(-c2ccc3c(c2)oc2ccccc23)n1]

Sub 2-2 (20 g, 44.2 mmol) and (4′-cyano-[1,1′-biphenyl]-3-yl)boronic acid (9.9 g, 44.2 mmol) were added to 400 ml of tetrahydrofuran under a nitrogen atmosphere, and the mixture was stirred and refluxed. Then, potassium carbonate (18.3 g, 132.7 mmol) was dissolved in 18 ml of water and added thereto, and the mixture was sufficiently stirred, and then tetrakistriphenyl-phosphinopalladium (1.5 g, 1.3 mmol) was added. After the reaction for 1 hour, the reaction mixture was cooled to room temperature, and then the organic layer and the aqueous layer were separated, and the organic layer was distilled. This was again added to 527 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 15 (19.2 g, 73%, MS: [M+H]+=596.2).

### Example 16: Preparation of Compound 16

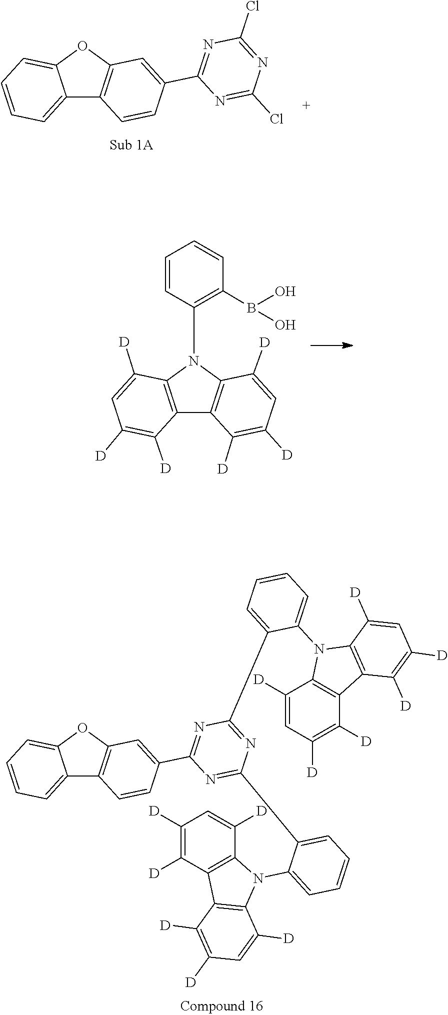 [SMILES: Clc1nc(Cl)nc(-c2ccc3c(c2)oc2ccccc23)n1 ; [2H]c1cc([2H])c2c(c1[2H])c1c([2H])c([2H])cc([2H])c1n2-c1ccccc1B(O)O ; [2H]c1cc([2H])c2c(c1[2H])c1c([2H])c([2H])cc([2H])c1n2-c1ccccc1-c1nc(-c2ccc3c(c2)oc2ccccc23)nc(-c2ccccc2-n2c3c([2H])cc([2H])c([2H])c3c3c([2H])c([2H])cc([2H])c32)n1]

Sub 1A (20 g, 63.5 mmol) and (2-(9H-carbazol-9-yl-1,3,4,5,6,8-d6)phenyl)boronic acid (39.1 g, 133.3 mmol) were added to 600 ml of tetrahydrofuran under a nitrogen atmosphere, and the mixture was stirred and refluxed. Then, potassium carbonate (26.3 g, 190.5 mmol) was dissolved in 26 ml of water and added thereto, and the mixture was sufficiently stirred, and then tetrakistriphenyl-phosphinopalladium (2.9 g, 2.5 mmol) was added. After the reaction for 2 hours, the reaction mixture was cooled to room temperature, and then the organic layer and the aqueous layer were separated, and the organic layer was distilled. This was again added to 941 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 16 (33.4 g, 71%, MS: [M+H]+=742.3).

### Example 17: Preparation of Compound 17

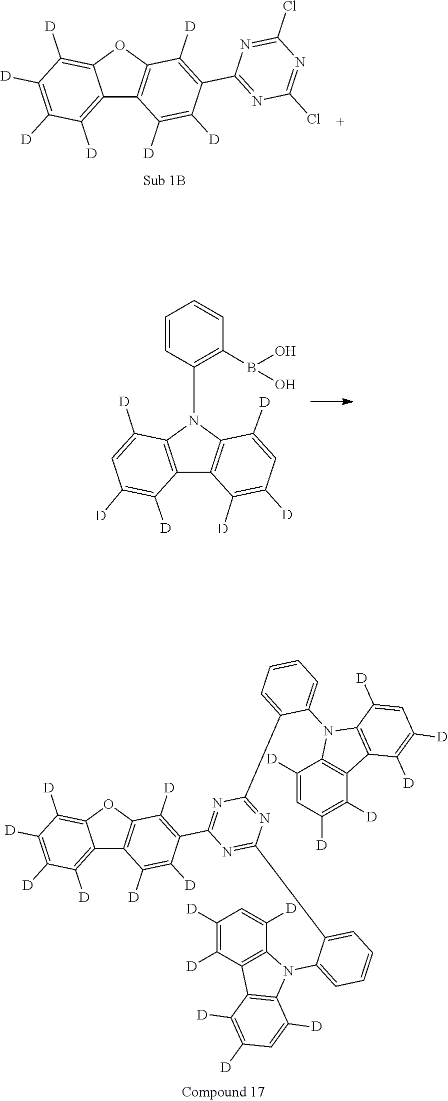 [SMILES: [2H]c1c([2H])c([2H])c2c(oc3c([2H])c(-c4nc(Cl)nc(Cl)n4)c([2H])c([2H])c32)c1[2H] ; [2H]c1cc([2H])c2c(c1[2H])c1c([2H])c([2H])cc([2H])c1n2-c1ccccc1B(O)O ; [2H]c1cc([2H])c2c(c1[2H])c1c([2H])c([2H])cc([2H])c1n2-c1ccccc1-c1nc(-c2ccccc2-n2c3c([2H])cc([2H])c([2H])c3c3c([2H])c([2H])cc([2H])c32)nc(-c2c([2H])c([2H])c3c(oc4c([2H])c([2H])c([2H])c([2H])c43)c2[2H])n1]

Sub 1B (20 g, 62.1 mmol) and (2-(9H-carbazol-9-yl-1,3,4,5,6,8-d6)phenyl)boronic acid (38.2 g, 130.4 mmol) were added to 600 ml of tetrahydrofuran under a nitrogen atmosphere, and the mixture was stirred and refluxed. Then, potassium carbonate (25.8 g, 186.3 mmol) was dissolved in 26 ml of water and added thereto, and the mixture was sufficiently stirred, and then tetrakistriphenyl-phosphinopalladium (2.9 g, 2.5 mmol) was added. After the reaction for 2 hours, the reaction mixture was cooled to room temperature, and then the organic layer and the aqueous layer were separated, and the organic layer was distilled. This was again added to 930 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 17 (27.4 g, 59%, MS: [M+H]+=749.4).

### Example 18: Preparation of Compound 18

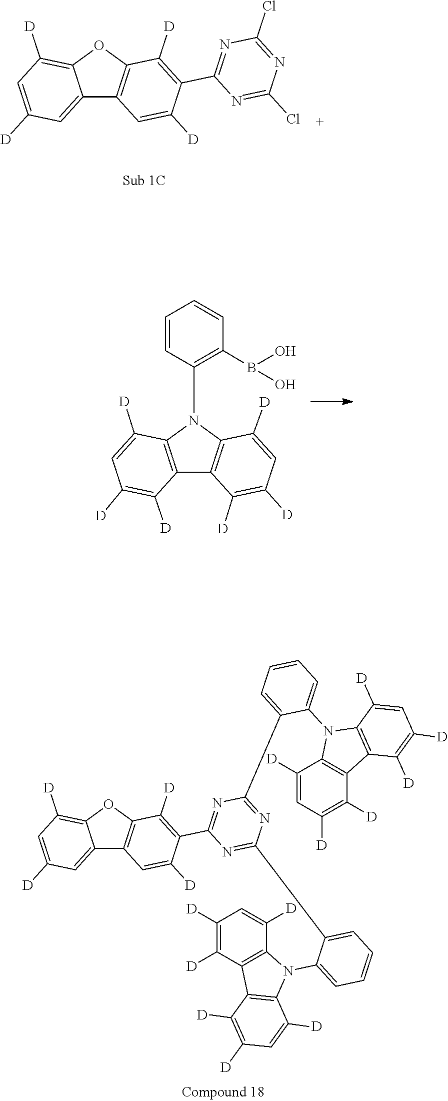 [SMILES: [2H]c1cc([2H])c2oc3c([2H])c(-c4nc(Cl)nc(Cl)n4)c([2H])cc3c2c1 ; [2H]c1cc([2H])c2c(c1[2H])c1c([2H])c([2H])cc([2H])c1n2-c1ccccc1B(O)O ; [2H]c1cc([2H])c2oc3c([2H])c(-c4nc(-c5ccccc5-n5c6c([2H])cc([2H])c([2H])c6c6c([2H])c([2H])cc([2H])c65)nc(-c5ccccc5-n5c6c([2H])cc([2H])c([2H])c6c6c([2H])c([2H])cc([2H])c65)n4)c([2H])cc3c2c1]

Sub 1C (20 g, 62.7 mmol) and (2-(9H-carbazol-9-yl-1,3,4,5,6,8-d6)phenyl)boronic acid (38.6 g, 131.7 mmol) were added to 600 ml of tetrahydrofuran under a nitrogen atmosphere, and the mixture was stirred and refluxed. Then, potassium carbonate (26 g, 188.1 mmol) was dissolved in 26 ml of water and added thereto, and the mixture was sufficiently stirred, and then tetrakistriphenyl-phosphinopalladium (2.9 g, 2.5 mmol) was added. After the reaction for 3 hours, the reaction mixture was cooled to room temperature, and then the organic layer and the aqueous layer were separated, and the organic layer was distilled. This was again added to 935 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 18 (36.9 g, 79%, MS: [M+H]+=746.4).

### Example 19: Preparation of Compound 19

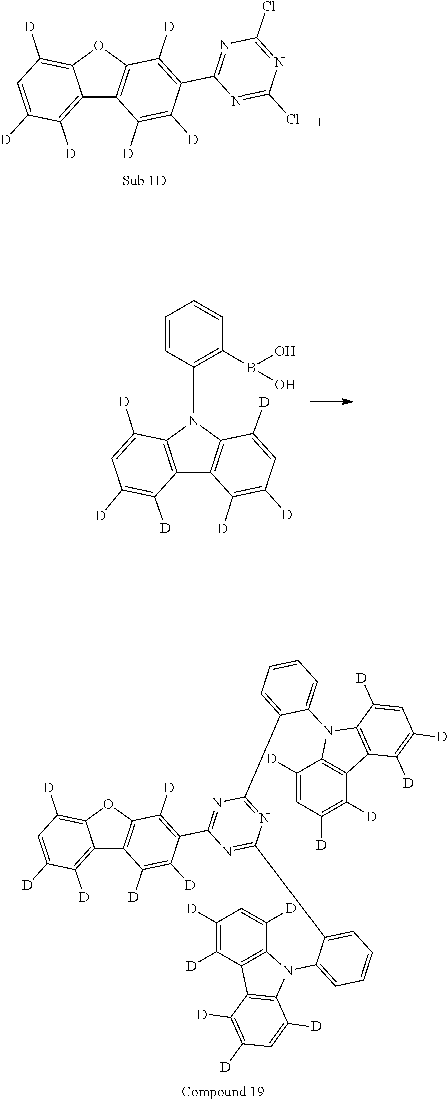 [SMILES: [2H]c1cc([2H])c2oc3c([2H])c(-c4nc(Cl)nc(Cl)n4)c([2H])c([2H])c3c2c1[2H] ; [2H]c1cc([2H])c2c(c1[2H])c1c([2H])c([2H])cc([2H])c1n2-c1ccccc1B(O)O ; [2H]c1cc([2H])c2oc3c([2H])c(-c4nc(-c5ccccc5-n5c6c([2H])cc([2H])c([2H])c6c6c([2H])c([2H])cc([2H])c65)nc(-c5ccccc5-n5c6c([2H])cc([2H])c([2H])c6c6c([2H])c([2H])cc([2H])c65)n4)c([2H])c([2H])c3c2c1[2H]]

Sub 1D (20 g, 62.3 mmol) and (2-(9H-carbazol-9-yl-1,3,4,5,6,8-d6)phenyl)boronic acid (38.4 g, 130.8 mmol) were added to 600 ml of tetrahydrofuran under a nitrogen atmosphere, and the mixture was stirred and refluxed. Then, potassium carbonate (25.8 g, 186.9 mmol) was dissolved in 26 ml of water and added thereto, and the mixture was sufficiently stirred, and then tetrakistriphenyl-phosphinopalladium (2.9 g, 2.5 mmol) was added. After the reaction for 3 hours, the reaction mixture was cooled to room temperature, and then the organic layer and the aqueous layer were separated, and the organic layer was distilled. This was again added to 931 mL of chloroform, dissolved, washed twice with water, and then the organic layer was separated, anhydrous magnesium sulfate was added, stirred, then filtered, and the filtrate was distilled under reduced pressure. The concentrated compound was recrystallized from chloroform and ethyl acetate to prepare a yellow solid compound 19 (30.7 g, 66%, MS: [M+H]+=748.4).

### EXPERIMENTAL EXAMPLE

### Experimental Example 1

A glass substrate on which ITO (indium tin oxide) was coated as a thin film to a thickness of 1,300 Å was put into distilled water in which a detergent was dissolved, and ultrasonically cleaned. At this time, a product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was cleaned with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was thermally vacuum-deposited to a thickness of 50 Å to form a hole injection layer. The following Compound HT-1 was thermally vacuum-deposited to a thickness of 250 Å on the hole injection layer to form a hole transport layer, and then, the following Compound HT-2 was vacuum-deposited to a thickness of 50 Å on the HT-1 deposited layer to form a hole blocking layer. Compound 1 prepared in the previous Example 1 as a light emitting layer, the following Compound YGH-1 and the phosphorescent dopant YGD-1 were co-deposited at a weight ratio of 44:44:12 on the HT-2 deposited layer to form a light emitting layer with a thickness of 400 Å. The following Compound ET-1 was vacuum-deposited to a thickness of 250 Å on the light emitting layer to form an electron transport layer. Then, the following Compound ET-2 and L₁ were vacuum-deposited at a ratio of 98:2 on the electron transport layer to form an electron injection layer with a thickness of 100 Å. Aluminum was deposited to a thickness of 1000 Å on the electron injection layer to form a cathode.

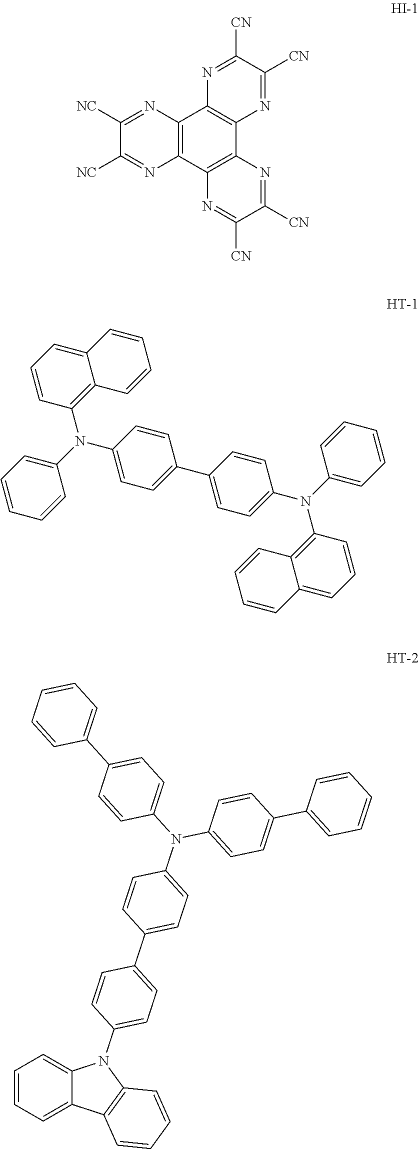 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; c1ccc(-c2ccc(N(c3ccc(-c4ccccc4)cc3)c3ccc(-c4ccc(-n5c6ccccc6c6ccccc65)cc4)cc3)cc2)cc1]

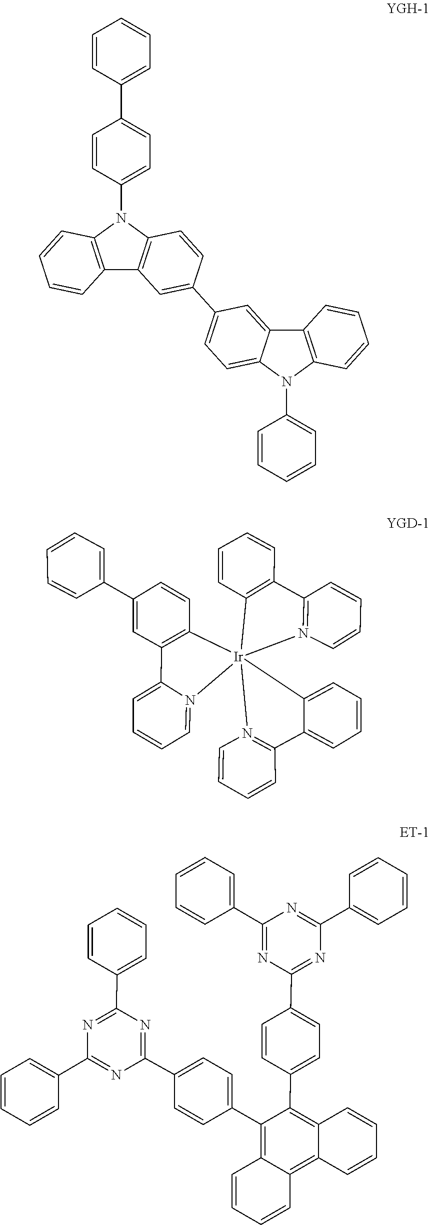

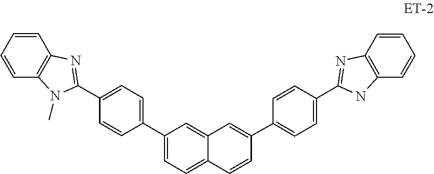

In the above-mentioned process, the vapor deposition rate of the organic material was maintained at 0.4˜0.7 Å/sec, the deposition rate of aluminum was maintained at 2 Å/sec, and the degree of vacuum during the deposition was maintained at 1×10⁻⁷˜5×10⁻⁸ torr.

### Experimental Examples 2 to 16

The organic light emitting devices were manufactured in the same manner as in Experimental Example 1, except that in Experimental Example 1, the compounds shown in Table 1 below were used instead of Compound 1 of Example 1.

### Comparative Experimental Examples 1 and 2

The organic light emitting devices were manufactured in the same manner as in Experimental Example 1, except that in Experimental Example 1, the compounds shown in Table 1 below were used instead of Compound 1 of Example 1. Compounds CE1 and CE2 of Table 1 below are as follows.

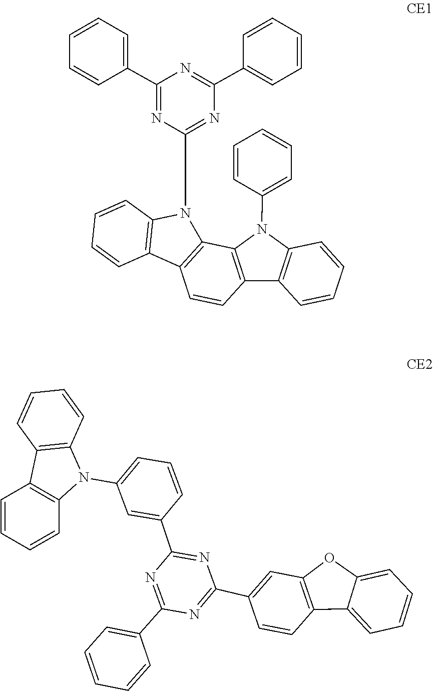 [SMILES: c1ccc(-c2nc(-c3ccccc3)nc(-n3c4ccccc4c4ccc5c6ccccc6n(-c6ccccc6)c5c43)n2)cc1 ; c1ccc(-c2nc(-c3cccc(-n4c5ccccc5c5ccccc54)c3)nc(-c3ccc4c(c3)oc3ccccc34)n2)cc1]

For the organic light emitting device manufactured in the Experimental Examples and Comparative Experimental Examples, the voltage and efficiency were measured at a current density of 10 mA/cm², the lifetime was measured at a current density of 50 mA/cm², and the results are shown in Table 1 below. At this time, LT95 means the time required for the luminance to be reduced to 95% of the initial luminance.

TABLE 1

| | | Voltage | Efficiency | | LT95 |
|---|---|---|---|---|---|
| | | (V@10 | (Cd/A@10 | Color | (hr@50 |
| | | mA/ | mA/ | coordinate | mA/ |
| | Compound | cm²) | cm²) | (x, y) | cm²) |
| Experimental | Compound | 4 | 75 | 0.46, 0.53 | 250 |
| Example 1 | 1 | | | | |
| Experimental | Compound | 4.1 | 75 | 0.46, 0.54 | 290 |
| Example 2 | 2 | | | | |
| Experimental | Compound | 4.2 | 74 | 0.44, 0.54 | 320 |
| Example 3 | 3 | | | | |
| Experimental | Compound | 3.9 | 76 | 0.46, 0.53 | 225 |
| Example 4 | 4 | | | | |
| Experimental | Compound | 4 | 77 | 0.46, 0.54 | 155 |
| Example 5 | 5 | | | | |
| Experimental | Compound | 4.1 | 72 | 0.46, 0.54 | 350 |
| Example 6 | 6 | | | | |
| Experimental | Compound | 3.9 | 77 | 0.46, 0.54 | 260 |
| Example 7 | 7 | | | | |
| Experimental | Compound | 4.1 | 74 | 0.46, 0.54 | 335 |
| Example 8 | 8 | | | | |
| Experimental | Compound | 4 | 76 | 0.46, 0.54 | 340 |
| Example 9 | 9 | | | | |
| Experimental | Compound | 4.1 | 73 | 0.46, 0.54 | 385 |
| Example 10 | 10 | | | | |
| Experimental | Compound | 4.2 | 74 | 0.46, 0.54 | 325 |
| Example 11 | 11 | | | | |
| Experimental | Compound | 4.1 | 72 | 0.46, 0.54 | 370 |
| Example 12 | 12 | | | | |
| Experimental | Compound | 4 | 75 | 0.46, 0.53 | 285 |
| Example 13 | 13 | | | | |
| Experimental | Compound | 4 | 75 | 0.46, 0.53 | 310 |
| Example 14 | 14 | | | | |
| Experimental | Compound | 4 | 75 | 0.46, 0.53 | 260 |
| Example 15 | 15 | | | | |
| Experimental | Compound | 4.2 | 74 | 0.46, 0.53 | 290 |
| Example 16 | 16 | | | | |
| Comparative | CE 1 | 4.3 | 65 | 0.45, 0.53 | 100 |
| Experimental | | | | | |
| Example 1 | | | | | |
| Comparative | CE 2 | 4.4 | 70 | 0.46, 0.53 | 145 |
| Experimental | | | | | |
| Example 2 | | | | | |

As shown in Table 1, it could be confirmed that when the compound of the present disclosure was used as the organic light emitting layer material, it exhibited excellent characteristics in terms of efficiency and lifetime as compared with Comparative Experimental Examples. This is considered to be because triazine and carbazole groups are substituted in the dibenzofuran group, which is a core substituent, thereby increasing electronic stability. In particular, when at least one deuterium is substituted in additional aryl group and carbazole group, it exhibits excellent properties of increasing the lifetime. This is also considered that the electronic stability is increased.

### DESCRIPTION OF REFERENCE NUMERALS

1: substrate
2: anode
3: light emitting layer
4: cathode
5: hole injection layer
6: hole transport layer
7: electron transport layer

1. A compound represented by the following Chemical Formula 1: 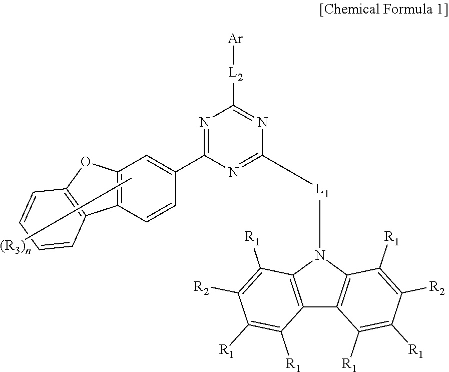
in Chemical Formula 1,
L₁ and L₂ are each independently a single bond, or a substituted or unsubstituted C₆₋₆₀ arylene,
Ar is a substituted or unsubstituted C₆₋₆₀ aryl, or a substituted or unsubstituted C₂₋₆₀ heteroaryl containing a heteroatom selected from the group consisting of N, O and S,
each R₁ is independently hydrogen or deuterium, with the proviso that at least one R₁ is deuterium,
each R₂ is independently hydrogen or deuterium,
each R₃ is independently hydrogen or deuterium, and
n is an integer of 0 to 7.

2. The compound according to claim 1, wherein
L₁ is a single bond; or phenylene.

3. The compound according to claim 1, wherein
L₂ is a single bond, phenylene, or naphthylene.

4. The compound according to claim 1, wherein
Ar is phenyl, biphenylyl, terphenylyl, naphthyl, phenanthrenyl, triphenylenyl, dibenzofuranyl, dibenzothiophenyl, carbazol-9-yl, 9-phenylcarbazolyl, or dimethylfluorenyl,
each of which is unsubstituted or substituted with at least one deuterium, cyano, or halogen.

5. The compound according to claim 1, wherein
at least two R₁s are deuterium.

6. The compound according to claim 1, wherein
all R₂s are hydrogen, or all R₂s are deuterium.

7. The compound according to claim 1, wherein
R₃ is deuterium, and
n is 0, 4, 6, or 7.

8. The compound according to claim 1, wherein:
the substituent 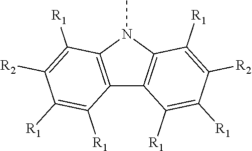
is one selected from the following:

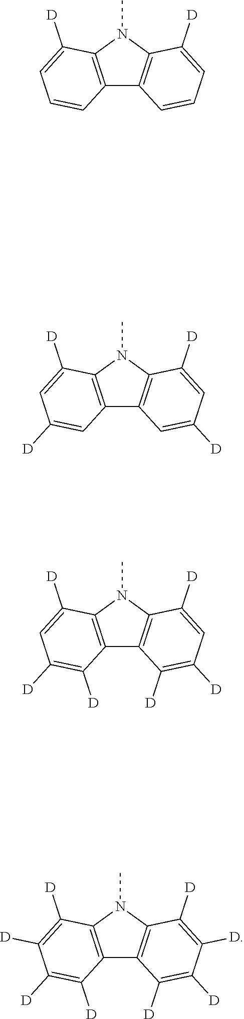

9. The compound according to claim 1, wherein
the compound represented by Chemical Formula 1 is one selected from the the following compounds:

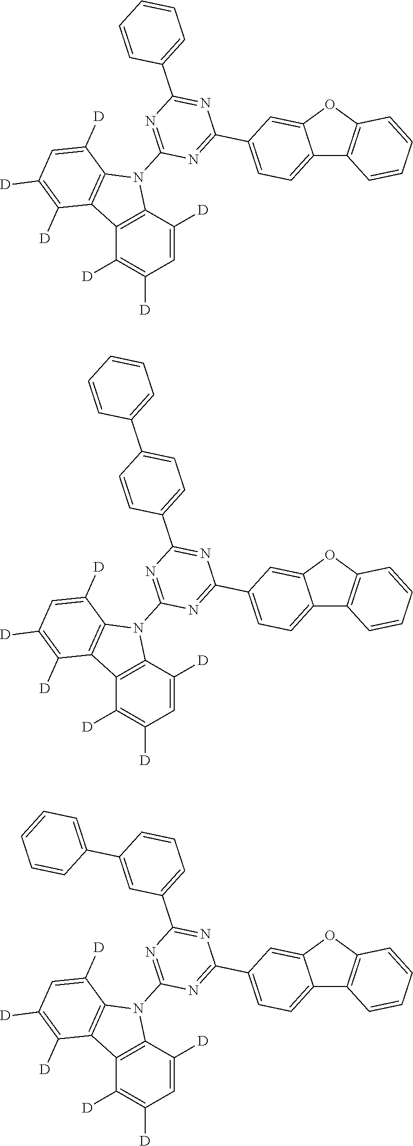 [SMILES: [2H]c1cc([2H])c2c(c1[2H])c1c([2H])c([2H])cc([2H])c1n2-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3ccccc3)cc2)nc(-c2ccc3c(c2)oc2ccccc23)n1 ; [2H]c1cc([2H])c2c(c1[2H])c1c([2H])c([2H])cc([2H])c1n2-c1nc(-c2cccc(-c3ccccc3)c2)nc(-c2ccc3c(c2)oc2ccccc23)n1]

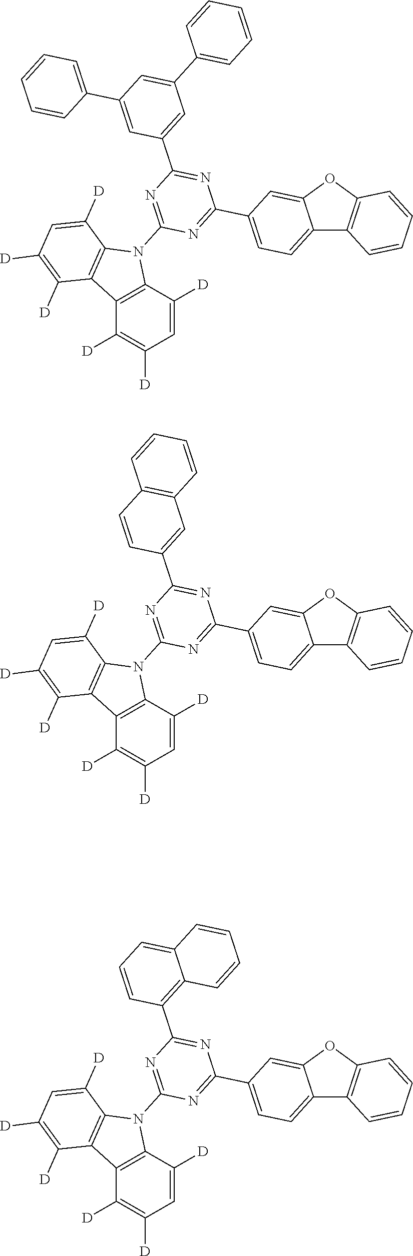 [SMILES: [2H]c1cc([2H])c2c(c1[2H])c1c([2H])c([2H])cc([2H])c1n2-c1nc(-c2cc(-c3ccccc3)cc(-c3ccccc3)c2)nc(-c2ccc3c(c2)oc2ccccc23)n1 ; [2H]c1cc([2H])c2c(c1[2H])c1c([2H])c([2H])cc([2H])c1n2-c1nc(-c2ccc3ccccc3c2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2cccc3ccccc23)n1]

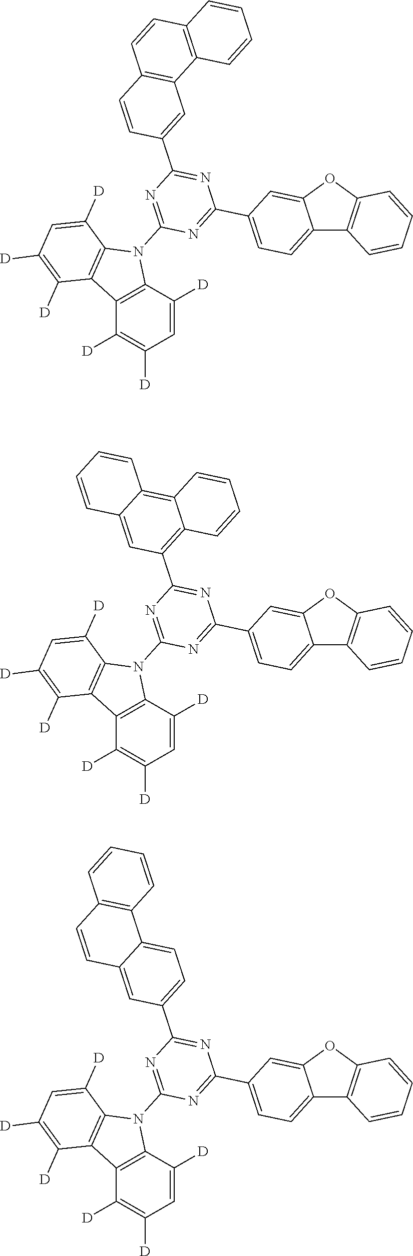 [SMILES: [2H]c1cc([2H])c2c(c1[2H])c1c([2H])c([2H])cc([2H])c1n2-c1nc(-c2ccc3c(c2)oc2ccccc23)nc(-c2ccc3ccc4ccccc4c3c2)n1 ; [2H]c1cc([2H])c2c(c1[2H])c1c([2H])c([2H])cc([2H])c1n2-c1nc(-c2ccc3c(c2)oc2ccccc23)nc(-c2cc3ccccc3c3ccccc23)n1 ; [2H]c1cc([2H])c2c(c1[2H])c1c([2H])c([2H])cc([2H])c1n2-c1nc(-c2ccc3c(ccc4ccccc43)c2)nc(-c2ccc3c(c2)oc2ccccc23)n1]

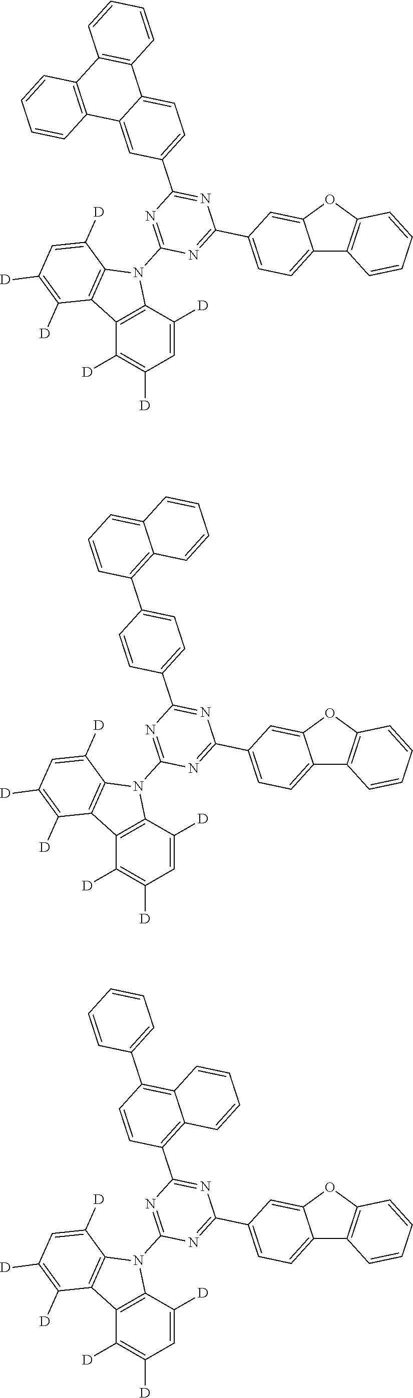 [SMILES: [2H]c1cc([2H])c2c(c1[2H])c1c([2H])c([2H])cc([2H])c1n2-c1nc(-c2ccc3c(c2)oc2ccccc23)nc(-c2ccc3c4ccccc4c4ccccc4c3c2)n1 ; [2H]c1cc([2H])c2c(c1[2H])c1c([2H])c([2H])cc([2H])c1n2-c1nc(-c2ccc(-c3cccc4ccccc34)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc(-c3ccccc3)c3ccccc23)n1]

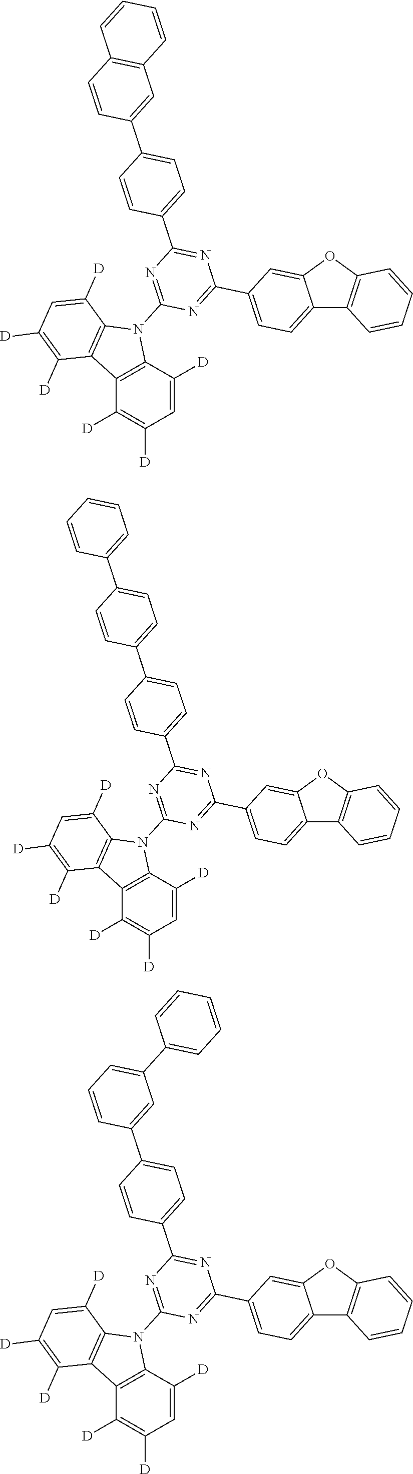

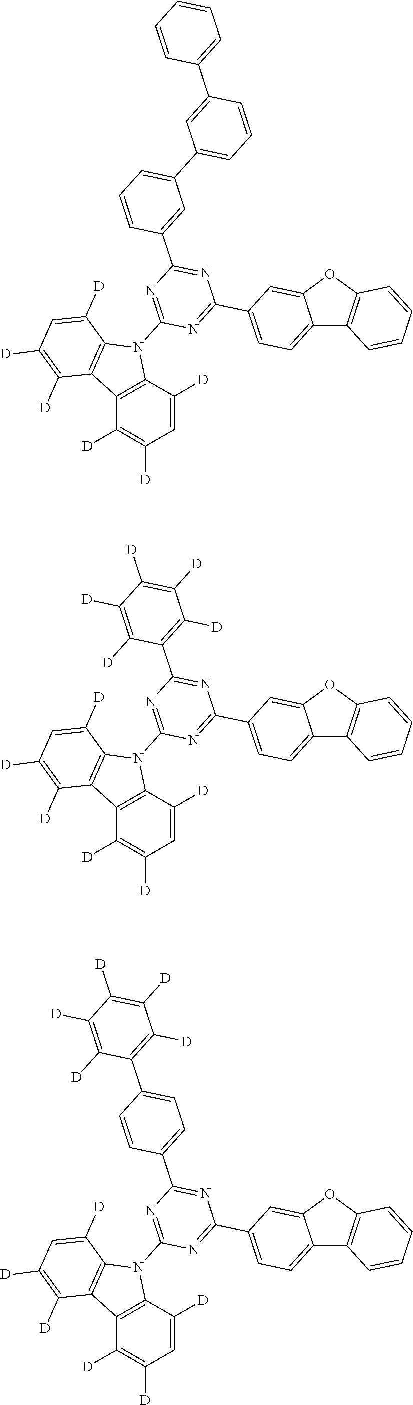 [SMILES: [2H]c1cc([2H])c2c(c1[2H])c1c([2H])c([2H])cc([2H])c1n2-c1nc(-c2cccc(-c3cccc(-c4ccccc4)c3)c2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2c([2H])c([2H])c([2H])c([2H])c2[2H])n1 ; [2H]c1cc([2H])c2c(c1[2H])c1c([2H])c([2H])cc([2H])c1n2-c1nc(-c2ccc(-c3c([2H])c([2H])c([2H])c([2H])c3[2H])cc2)nc(-c2ccc3c(c2)oc2ccccc23)n1]

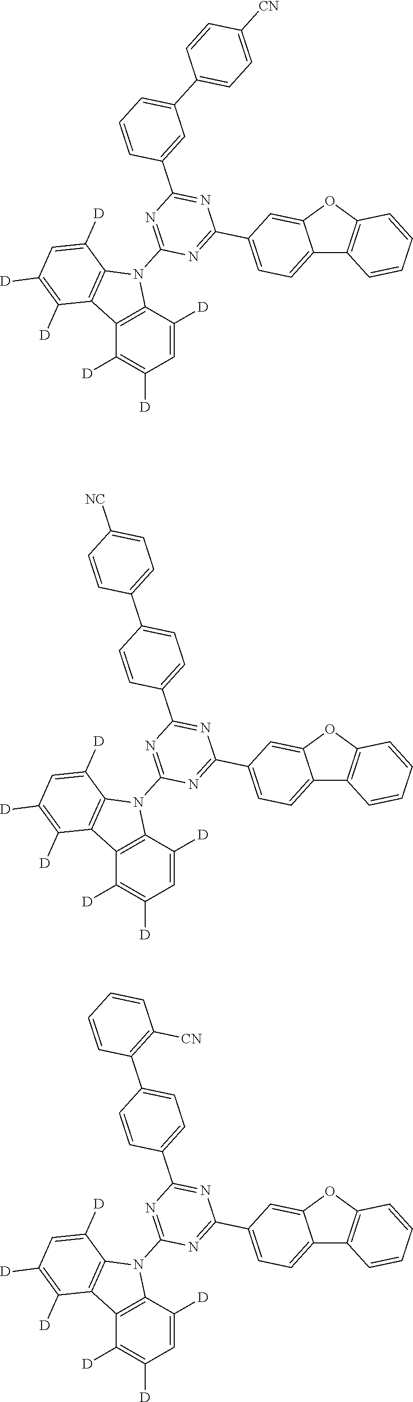 [SMILES: [2H]c1cc([2H])c2c(c1[2H])c1c([2H])c([2H])cc([2H])c1n2-c1nc(-c2cccc(-c3ccc(C#N)cc3)c2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3ccc(C#N)cc3)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc(-c3ccccc3C#N)cc2)nc(-c2ccc3c(c2)oc2ccccc23)n1]

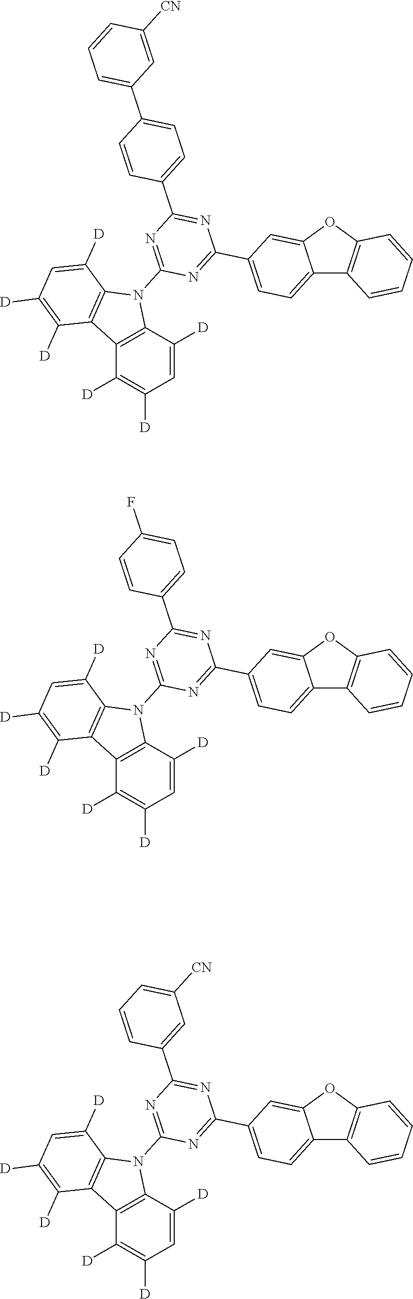 [SMILES: [2H]c1cc([2H])c2c(c1[2H])c1c([2H])c([2H])cc([2H])c1n2-c1nc(-c2ccc(-c3cccc(C#N)c3)cc2)nc(-c2ccc3c(c2)oc2ccccc23)n1 ; [2H]c1cc([2H])c2c(c1[2H])c1c([2H])c([2H])cc([2H])c1n2-c1nc(-c2ccc(F)cc2)nc(-c2ccc3c(c2)oc2ccccc23)n1 ; [2H]c1cc([2H])c2c(c1[2H])c1c([2H])c([2H])cc([2H])c1n2-c1nc(-c2cccc(C#N)c2)nc(-c2ccc3c(c2)oc2ccccc23)n1]

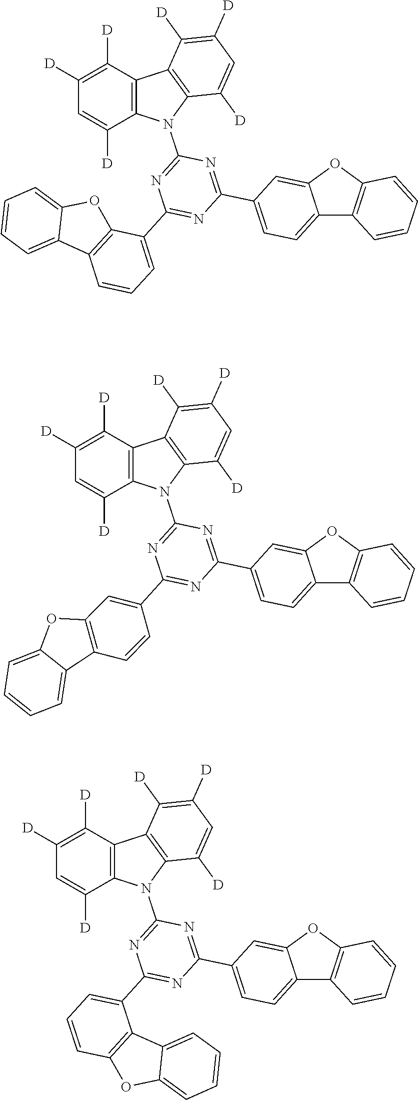 [SMILES: [2H]c1cc([2H])c2c(c1[2H])c1c([2H])c([2H])cc([2H])c1n2-c1nc(-c2ccc3c(c2)oc2ccccc23)nc(-c2cccc3c2oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2cccc3oc4ccccc4c23)n1]

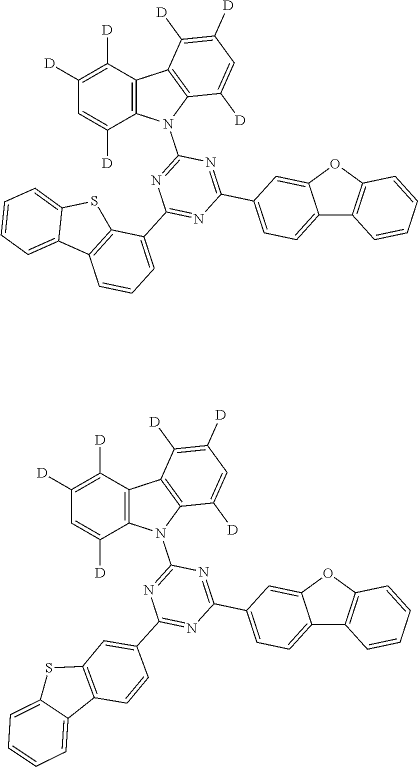 [SMILES: [2H]c1cc([2H])c2c(c1[2H])c1c([2H])c([2H])cc([2H])c1n2-c1nc(-c2ccc3c(c2)oc2ccccc23)nc(-c2cccc3c2sc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccc3c(c2)sc2ccccc23)n1]

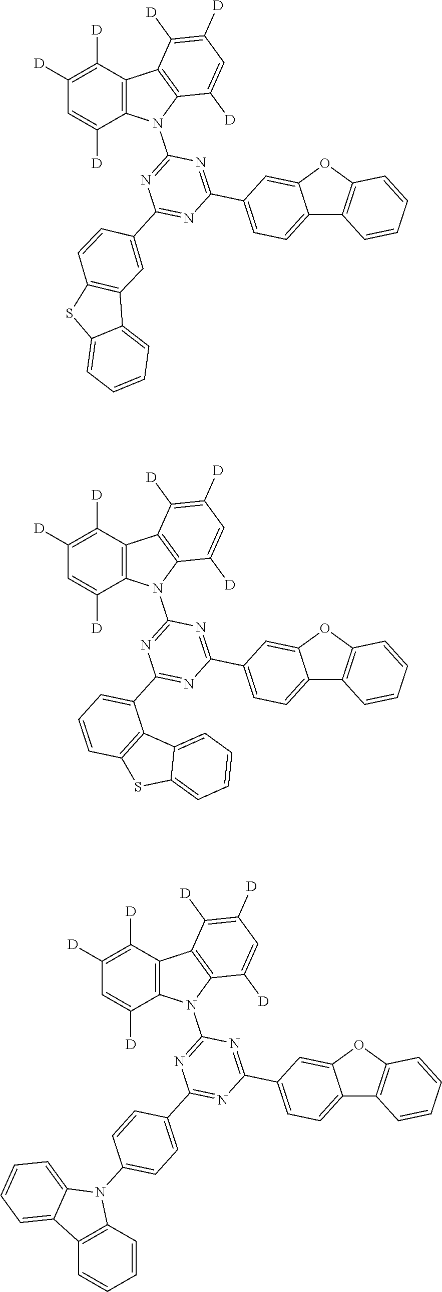 [SMILES: [2H]c1cc([2H])c2c(c1[2H])c1c([2H])c([2H])cc([2H])c1n2-c1nc(-c2ccc3c(c2)oc2ccccc23)nc(-c2ccc3sc4ccccc4c3c2)n1 ; [2H]c1cc([2H])c2c(c1[2H])c1c([2H])c([2H])cc([2H])c1n2-c1nc(-c2ccc3c(c2)oc2ccccc23)nc(-c2cccc3sc4ccccc4c23)n1 ; [2H]c1cc([2H])c2c(c1[2H])c1c([2H])c([2H])cc([2H])c1n2-c1nc(-c2ccc(-n3c4ccccc4c4ccccc43)cc2)nc(-c2ccc3c(c2)oc2ccccc23)n1]

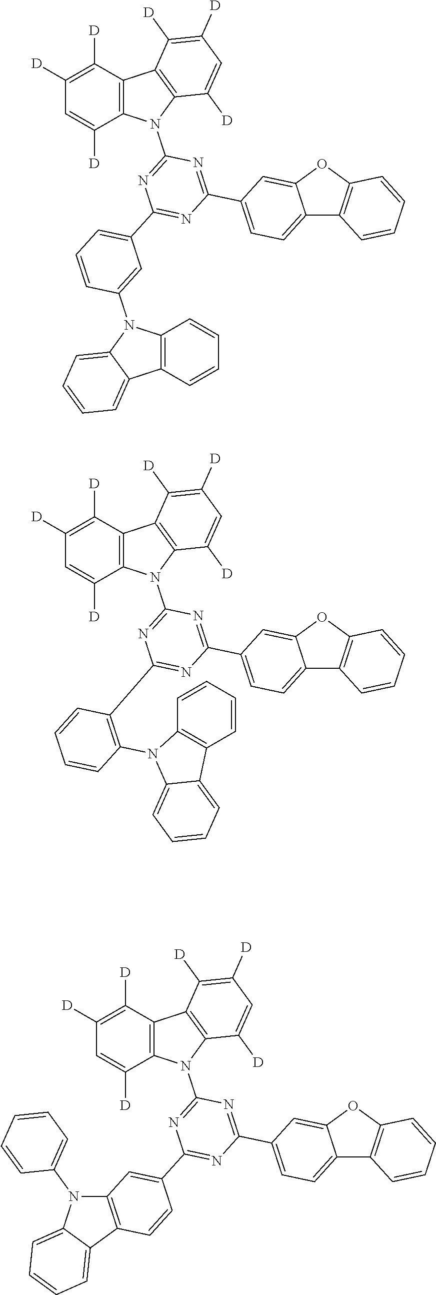 [SMILES: [2H]c1cc([2H])c2c(c1[2H])c1c([2H])c([2H])cc([2H])c1n2-c1nc(-c2cccc(-n3c4ccccc4c4ccccc43)c2)nc(-c2ccc3c(c2)oc2ccccc23)n1 ; [2H]c1cc([2H])c2c(c1[2H])c1c([2H])c([2H])cc([2H])c1n2-c1nc(-c2ccc3c(c2)oc2ccccc23)nc(-c2ccccc2-n2c3ccccc3c3ccccc32)n1 ; [2H]c1cc([2H])c2c(c1[2H])c1c([2H])c([2H])cc([2H])c1n2-c1nc(-c2ccc3c(c2)oc2ccccc23)nc(-c2ccc3c4ccccc4n(-c4ccccc4)c3c2)n1]

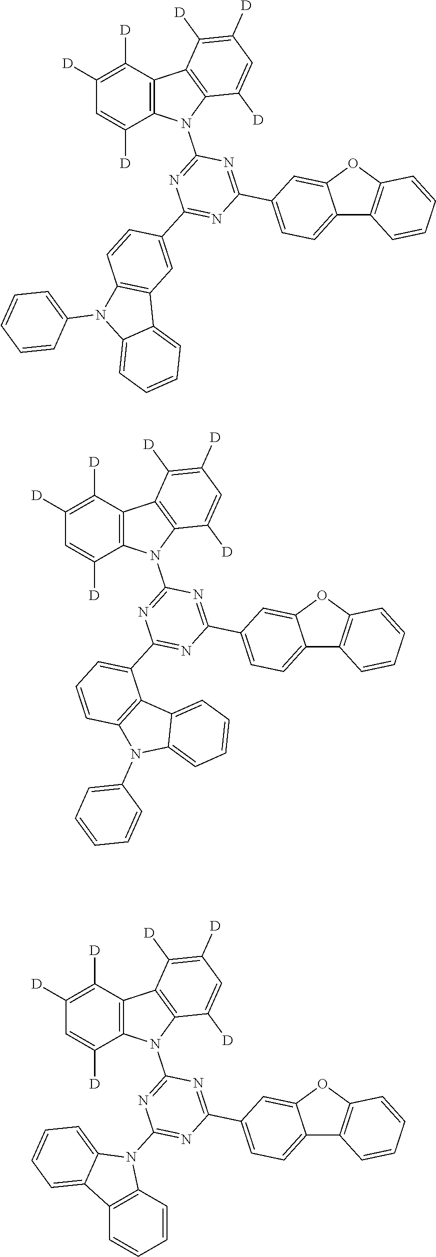 [SMILES: [2H]c1cc([2H])c2c(c1[2H])c1c([2H])c([2H])cc([2H])c1n2-c1nc(-c2ccc3c(c2)oc2ccccc23)nc(-c2ccc3c(c2)c2ccccc2n3-c2ccccc2)n1 ; [2H]c1cc([2H])c2c(c1[2H])c1c([2H])c([2H])cc([2H])c1n2-c1nc(-c2ccc3c(c2)oc2ccccc23)nc(-c2cccc3c2c2ccccc2n3-c2ccccc2)n1 ; [2H]c1cc([2H])c2c(c1[2H])c1c([2H])c([2H])cc([2H])c1n2-c1nc(-c2ccc3c(c2)oc2ccccc23)nc(-n2c3ccccc3c3ccccc32)n1]

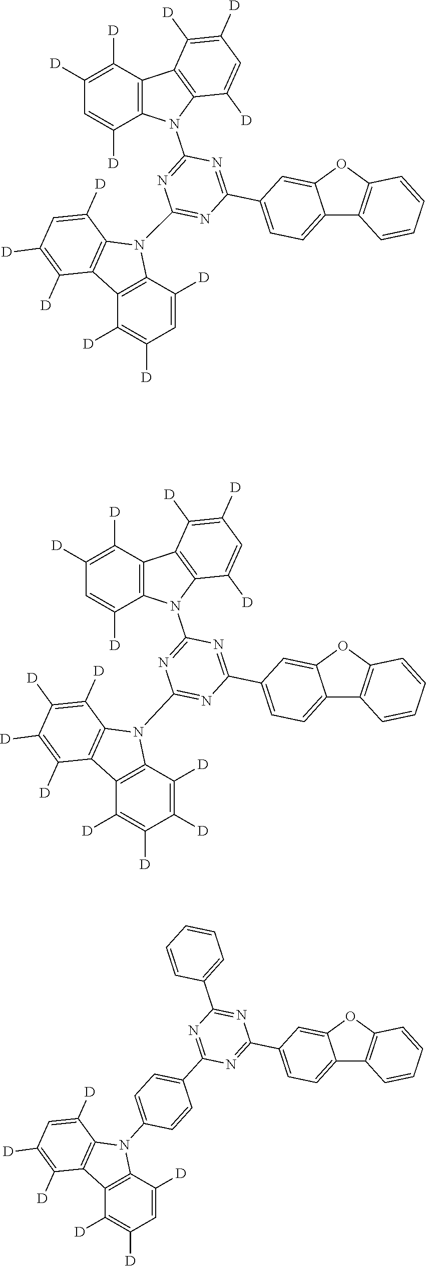 [SMILES: [2H]c1cc([2H])c2c(c1[2H])c1c([2H])c([2H])cc([2H])c1n2-c1nc(-c2ccc3c(c2)oc2ccccc23)nc(-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1nc(-c2ccc3c(c2)oc2ccccc23)nc(-n2c3c([2H])c([2H])c([2H])c([2H])c3c3c([2H])c([2H])c([2H])c([2H])c32)n1 ; [2H]c1cc([2H])c2c(c1[2H])c1c([2H])c([2H])cc([2H])c1n2-c1ccc(-c2nc(-c3ccccc3)nc(-c3ccc4c(c3)oc3ccccc34)n2)cc1]

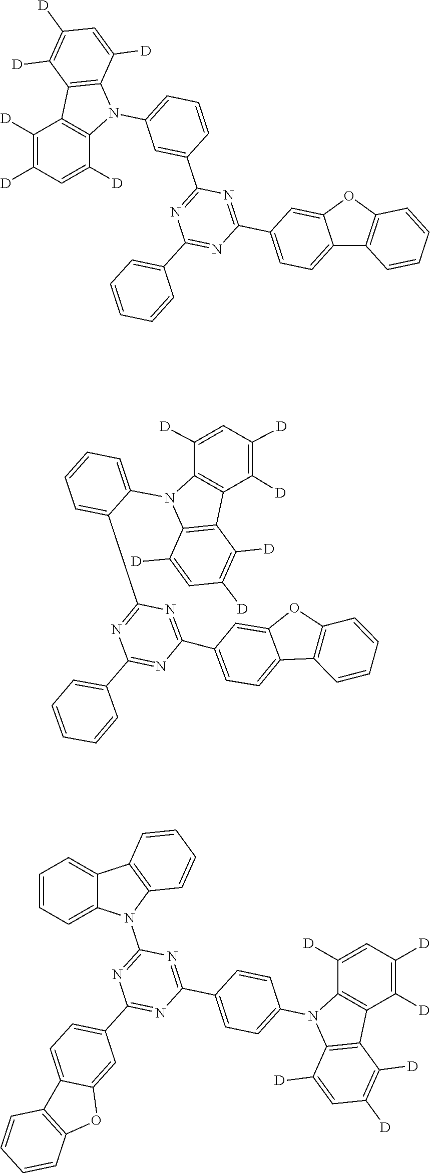 [SMILES: [2H]c1cc([2H])c2c(c1[2H])c1c([2H])c([2H])cc([2H])c1n2-c1cccc(-c2nc(-c3ccccc3)nc(-c3ccc4c(c3)oc3ccccc34)n2)c1 ; [2H]c1cc([2H])c2c(c1[2H])c1c([2H])c([2H])cc([2H])c1n2-c1ccccc1-c1nc(-c2ccccc2)nc(-c2ccc3c(c2)oc2ccccc23)n1 ; [2H]c1cc([2H])c2c(c1[2H])c1c([2H])c([2H])cc([2H])c1n2-c1ccc(-c2nc(-c3ccc4c(c3)oc3ccccc34)nc(-n3c4ccccc4c4ccccc43)n2)cc1]

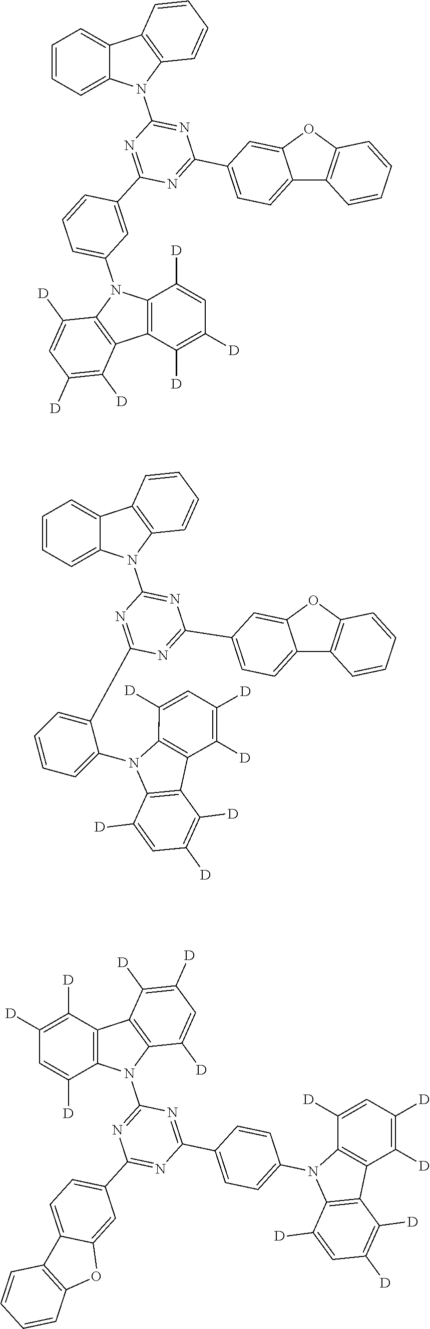 [SMILES: [2H]c1cc([2H])c2c(c1[2H])c1c([2H])c([2H])cc([2H])c1n2-c1cccc(-c2nc(-c3ccc4c(c3)oc3ccccc34)nc(-n3c4ccccc4c4ccccc43)n2)c1 ; [2H]c1cc([2H])c2c(c1[2H])c1c([2H])c([2H])cc([2H])c1n2-c1ccccc1-c1nc(-c2ccc3c(c2)oc2ccccc23)nc(-n2c3ccccc3c3ccccc32)n1 ; [2H]c1cc([2H])c2c(c1[2H])c1c([2H])c([2H])cc([2H])c1n2-c1ccc(-c2nc(-c3ccc4c(c3)oc3ccccc34)nc(-n3c4c([2H])cc([2H])c([2H])c4c4c([2H])c([2H])cc([2H])c43)n2)cc1]

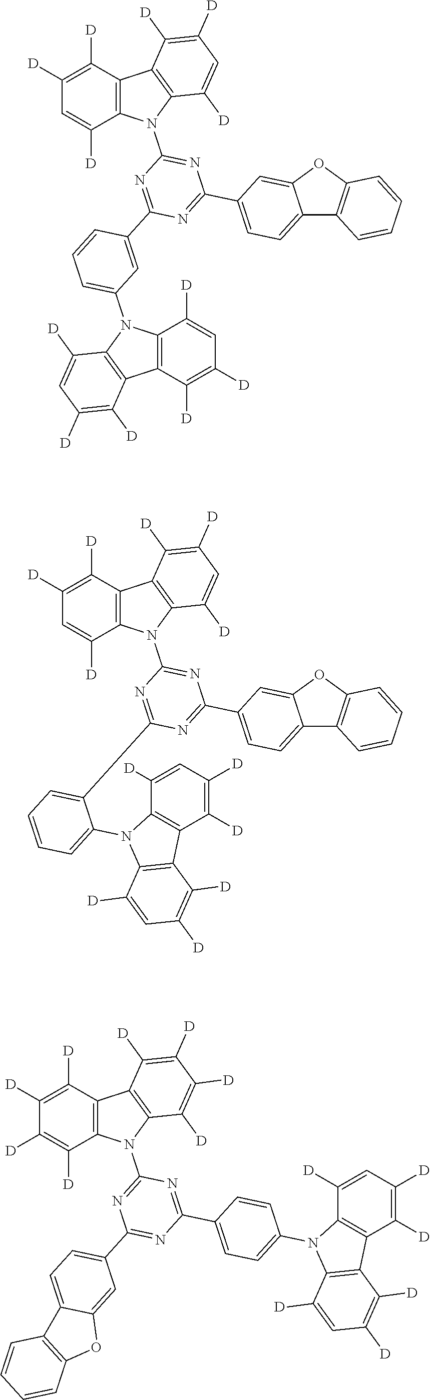 [SMILES: [2H]c1cc([2H])c2c(c1[2H])c1c([2H])c([2H])cc([2H])c1n2-c1cccc(-c2nc(-c3ccc4c(c3)oc3ccccc34)nc(-n3c4c([2H])cc([2H])c([2H])c4c4c([2H])c([2H])cc([2H])c43)n2)c1 ; [2H]c1cc([2H])c2c(c1[2H])c1c([2H])c([2H])cc([2H])c1n2-c1nc(-c2ccc3c(c2)oc2ccccc23)nc(-c2ccccc2-n2c3c([2H])cc([2H])c([2H])c3c3c([2H])c([2H])cc([2H])c32)n1 ; [2H]c1cc([2H])c2c(c1[2H])c1c([2H])c([2H])cc([2H])c1n2-c1ccc(-c2nc(-c3ccc4c(c3)oc3ccccc34)nc(-n3c4c([2H])c([2H])c([2H])c([2H])c4c4c([2H])c([2H])c([2H])c([2H])c43)n2)cc1]

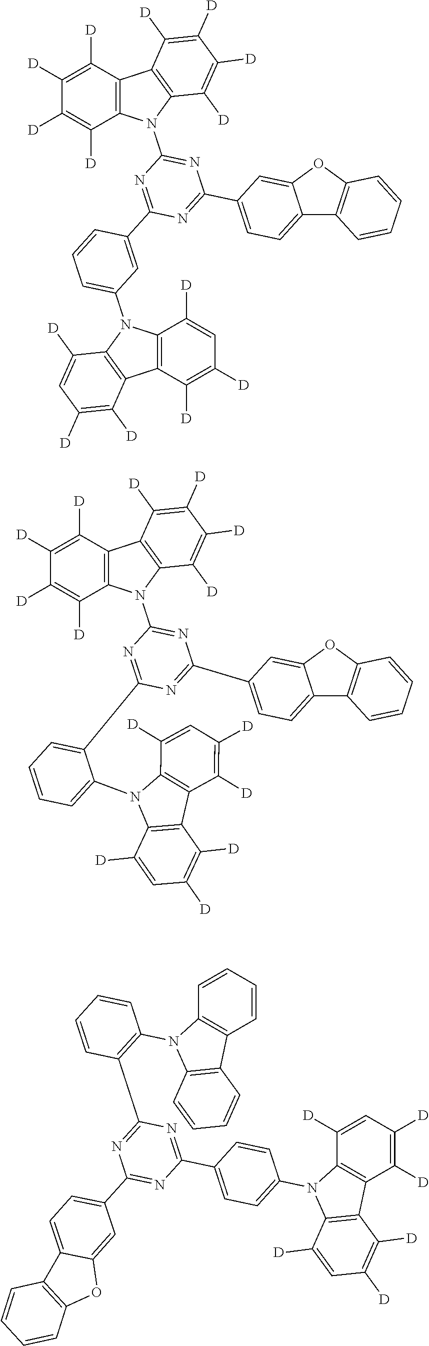 [SMILES: [2H]c1cc([2H])c2c(c1[2H])c1c([2H])c([2H])cc([2H])c1n2-c1cccc(-c2nc(-c3ccc4c(c3)oc3ccccc34)nc(-n3c4c([2H])c([2H])c([2H])c([2H])c4c4c([2H])c([2H])c([2H])c([2H])c43)n2)c1 ; [2H]c1cc([2H])c2c(c1[2H])c1c([2H])c([2H])cc([2H])c1n2-c1ccccc1-c1nc(-c2ccc3c(c2)oc2ccccc23)nc(-n2c3c([2H])c([2H])c([2H])c([2H])c3c3c([2H])c([2H])c([2H])c([2H])c32)n1 ; [2H]c1cc([2H])c2c(c1[2H])c1c([2H])c([2H])cc([2H])c1n2-c1ccc(-c2nc(-c3ccc4c(c3)oc3ccccc34)nc(-c3ccccc3-n3c4ccccc4c4ccccc43)n2)cc1]

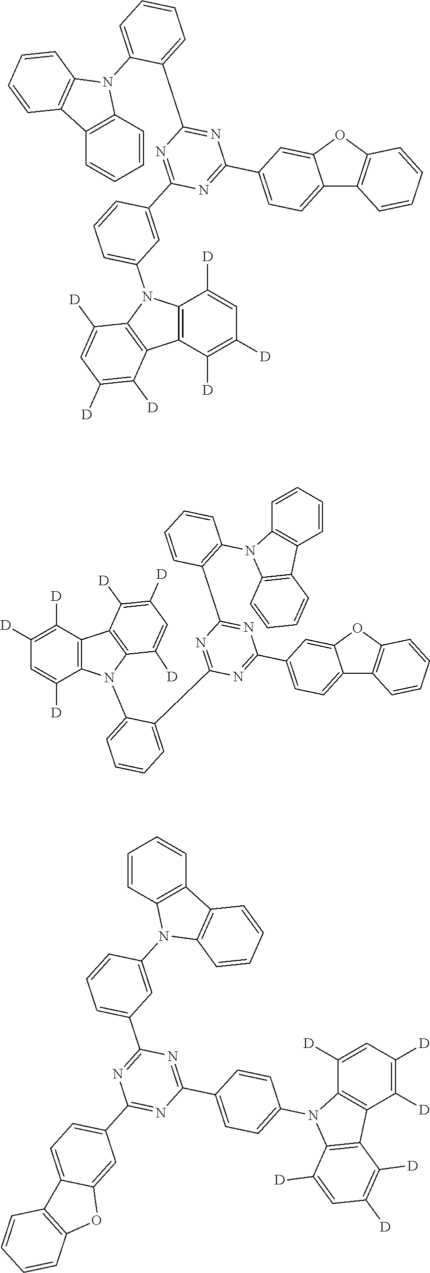 [SMILES: [2H]c1cc([2H])c2c(c1[2H])c1c([2H])c([2H])cc([2H])c1n2-c1cccc(-c2nc(-c3ccc4c(c3)oc3ccccc34)nc(-c3ccccc3-n3c4ccccc4c4ccccc43)n2)c1 ; [2H]c1cc([2H])c2c(c1[2H])c1c([2H])c([2H])cc([2H])c1n2-c1ccccc1-c1nc(-c2ccc3c(c2)oc2ccccc23)nc(-c2ccccc2-n2c3ccccc3c3ccccc32)n1 ; [2H]c1cc([2H])c2c(c1[2H])c1c([2H])c([2H])cc([2H])c1n2-c1ccc(-c2nc(-c3cccc(-n4c5ccccc5c5ccccc54)c3)nc(-c3ccc4c(c3)oc3ccccc34)n2)cc1]

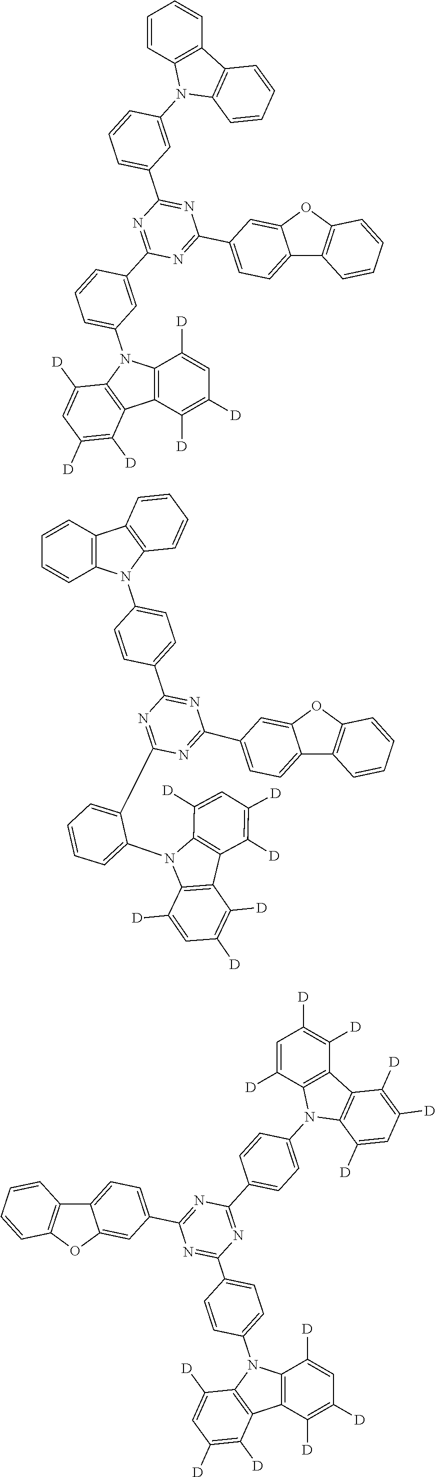

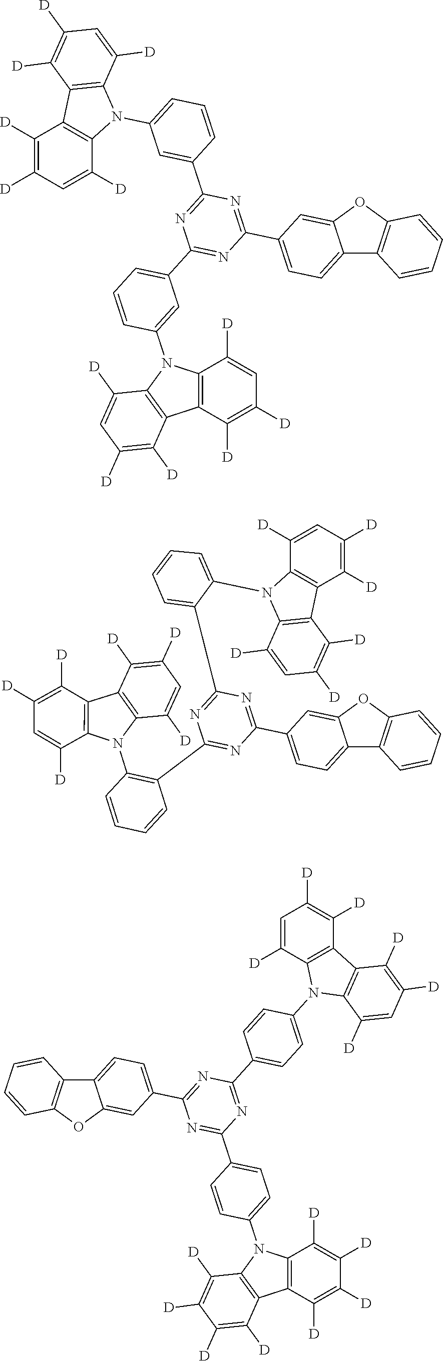

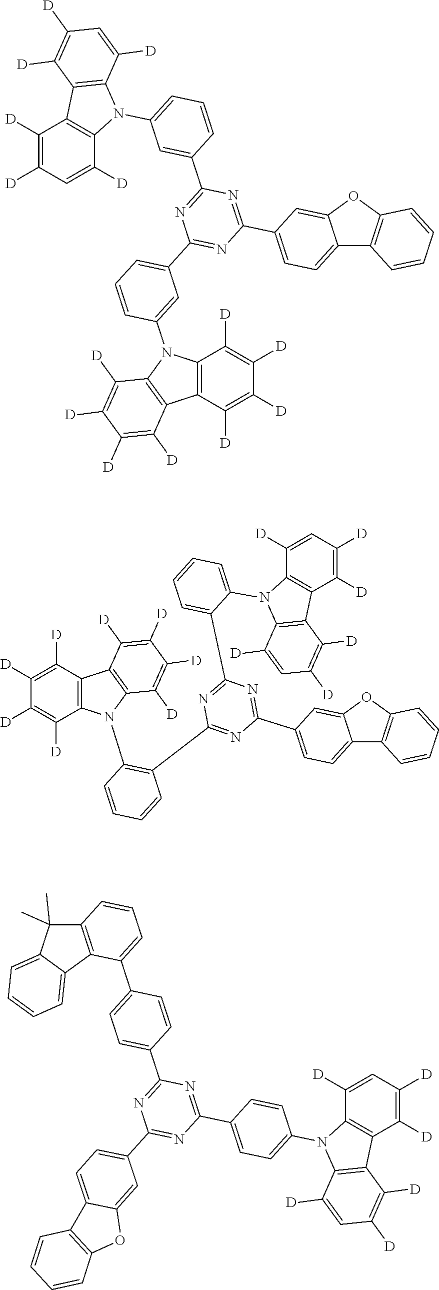 [SMILES: [2H]c1cc([2H])c2c(c1[2H])c1c([2H])c([2H])cc([2H])c1n2-c1cccc(-c2nc(-c3cccc(-n4c5c([2H])c([2H])c([2H])c([2H])c5c5c([2H])c([2H])c([2H])c([2H])c54)c3)nc(-c3ccc4c(c3)oc3ccccc34)n2)c1 ; [2H]c1cc([2H])c2c(c1[2H])c1c([2H])c([2H])cc([2H])c1n2-c1ccccc1-c1nc(-c2ccc3c(c2)oc2ccccc23)nc(-c2ccccc2-n2c3c([2H])c([2H])c([2H])c([2H])c3c3c([2H])c([2H])c([2H])c([2H])c32)n1 ; [2H]c1cc([2H])c2c(c1[2H])c1c([2H])c([2H])cc([2H])c1n2-c1ccc(-c2nc(-c3ccc(-c4cccc5c4-c4ccccc4C5(C)C)cc3)nc(-c3ccc4c(c3)oc3ccccc34)n2)cc1]

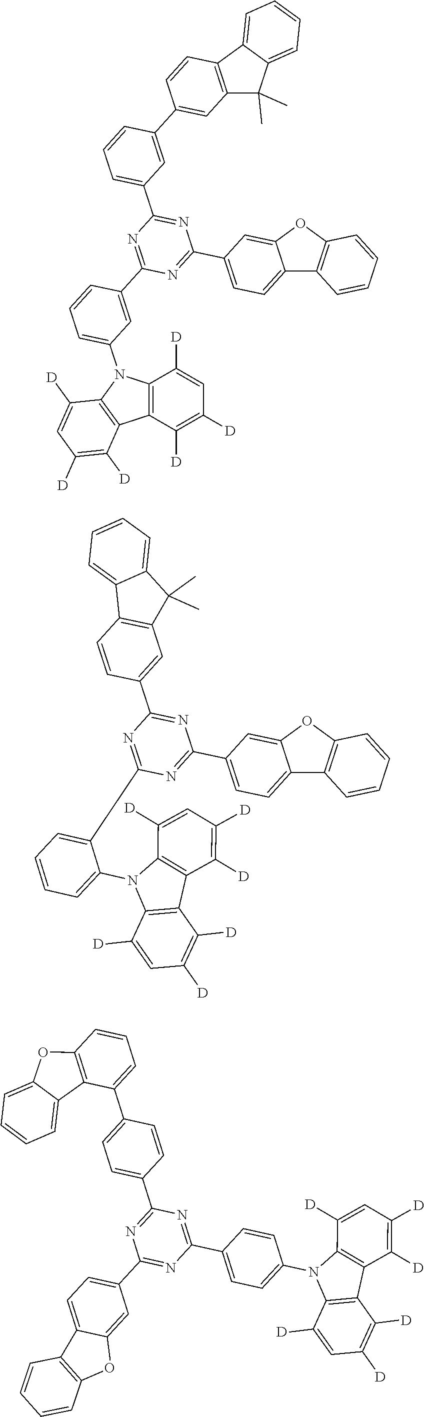

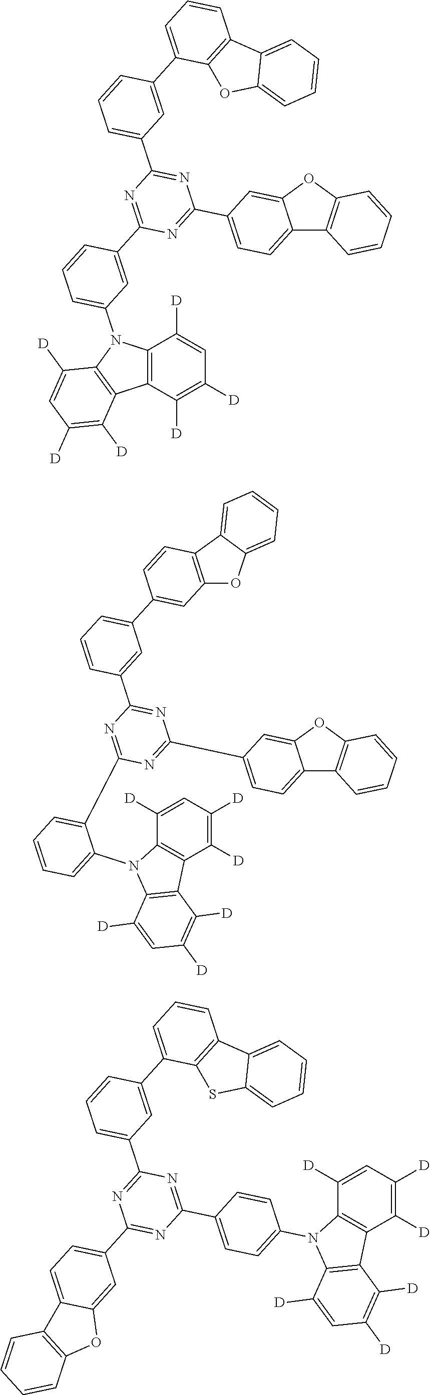

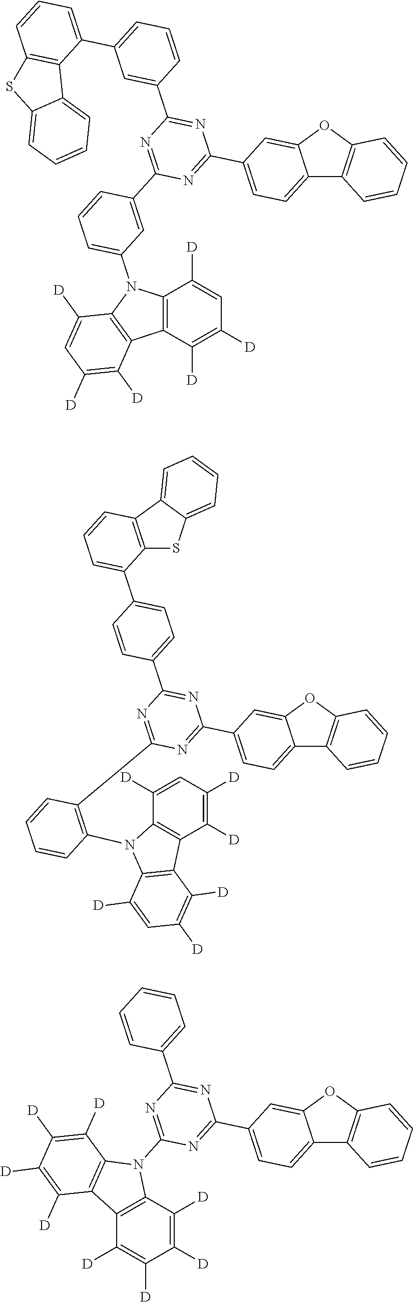 [SMILES: [2H]c1cc([2H])c2c(c1[2H])c1c([2H])c([2H])cc([2H])c1n2-c1cccc(-c2nc(-c3cccc(-c4cccc5sc6ccccc6c45)c3)nc(-c3ccc4c(c3)oc3ccccc34)n2)c1 ; [2H]c1cc([2H])c2c(c1[2H])c1c([2H])c([2H])cc([2H])c1n2-c1ccccc1-c1nc(-c2ccc(-c3cccc4c3sc3ccccc34)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccccc2)nc(-c2ccc3c(c2)oc2ccccc23)n1]

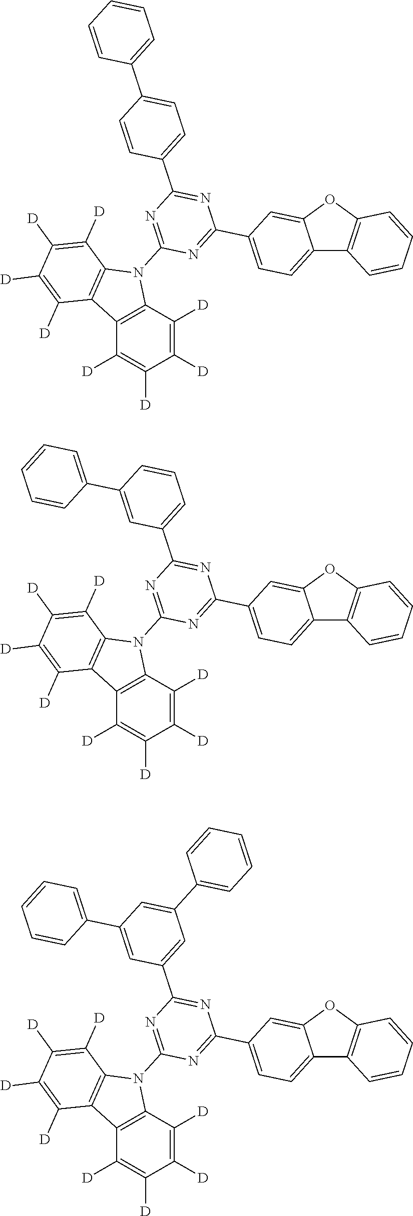 [SMILES: [2H]c1c([2H])c([2H])c2c(c1[2H])c1c([2H])c([2H])c([2H])c([2H])c1n2-c1nc(-c2ccc(-c3ccccc3)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2cccc(-c3ccccc3)c2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2cc(-c3ccccc3)cc(-c3ccccc3)c2)nc(-c2ccc3c(c2)oc2ccccc23)n1]

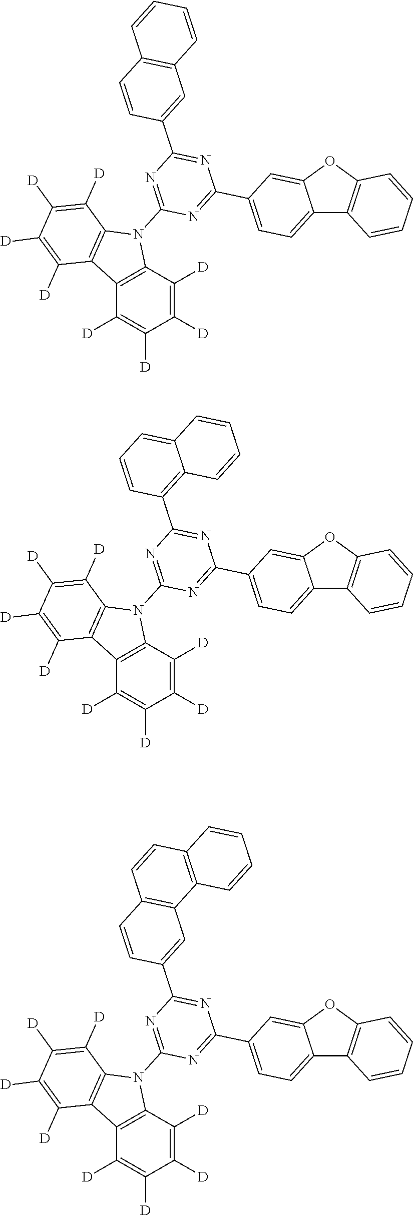 [SMILES: [2H]c1c([2H])c([2H])c2c(c1[2H])c1c([2H])c([2H])c([2H])c([2H])c1n2-c1nc(-c2ccc3ccccc3c2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2cccc3ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2ccc3ccc4ccccc4c3c2)n1]

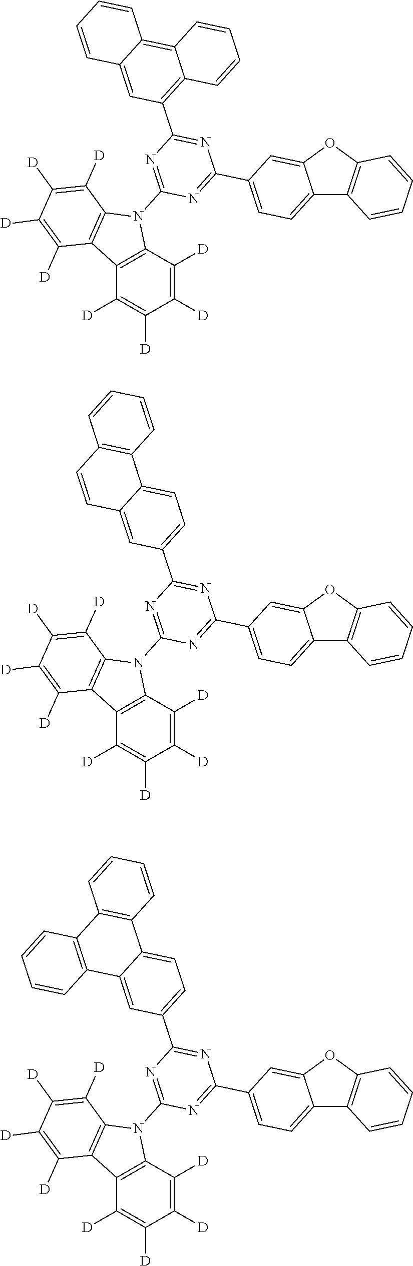 [SMILES: [2H]c1c([2H])c([2H])c2c(c1[2H])c1c([2H])c([2H])c([2H])c([2H])c1n2-c1nc(-c2ccc3c(c2)oc2ccccc23)nc(-c2cc3ccccc3c3ccccc23)n1 ; [2H]c1c([2H])c([2H])c2c(c1[2H])c1c([2H])c([2H])c([2H])c([2H])c1n2-c1nc(-c2ccc3c(ccc4ccccc43)c2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2ccc3c4ccccc4c4ccccc4c3c2)n1]

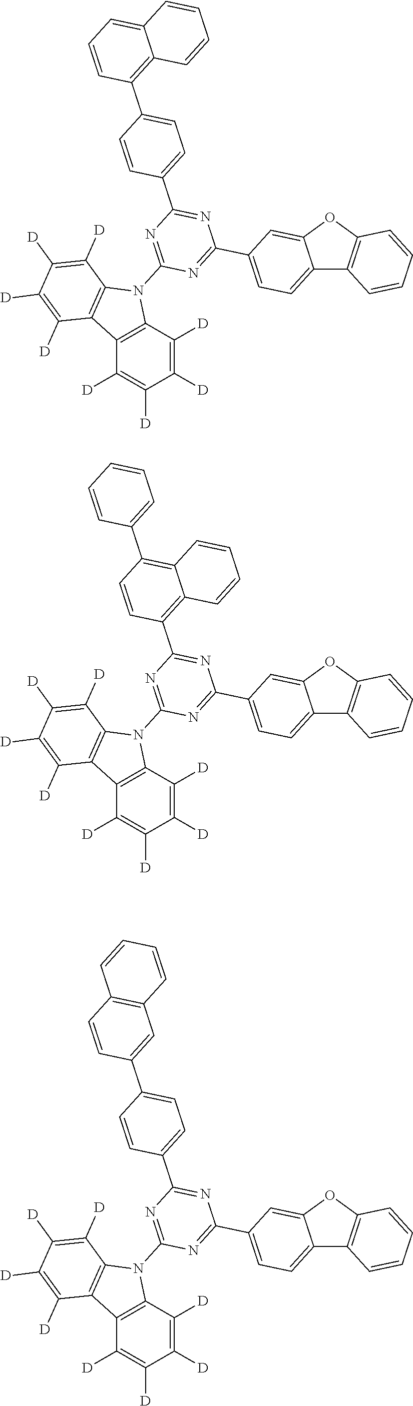 [SMILES: [2H]c1c([2H])c([2H])c2c(c1[2H])c1c([2H])c([2H])c([2H])c([2H])c1n2-c1nc(-c2ccc(-c3cccc4ccccc34)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2ccc3c(c2)oc2ccccc23)nc(-c2ccc(-c3ccccc3)c3ccccc23)n1 ; [2H]c1c([2H])c([2H])c2c(c1[2H])c1c([2H])c([2H])c([2H])c([2H])c1n2-c1nc(-c2ccc(-c3ccc4ccccc4c3)cc2)nc(-c2ccc3c(c2)oc2ccccc23)n1]

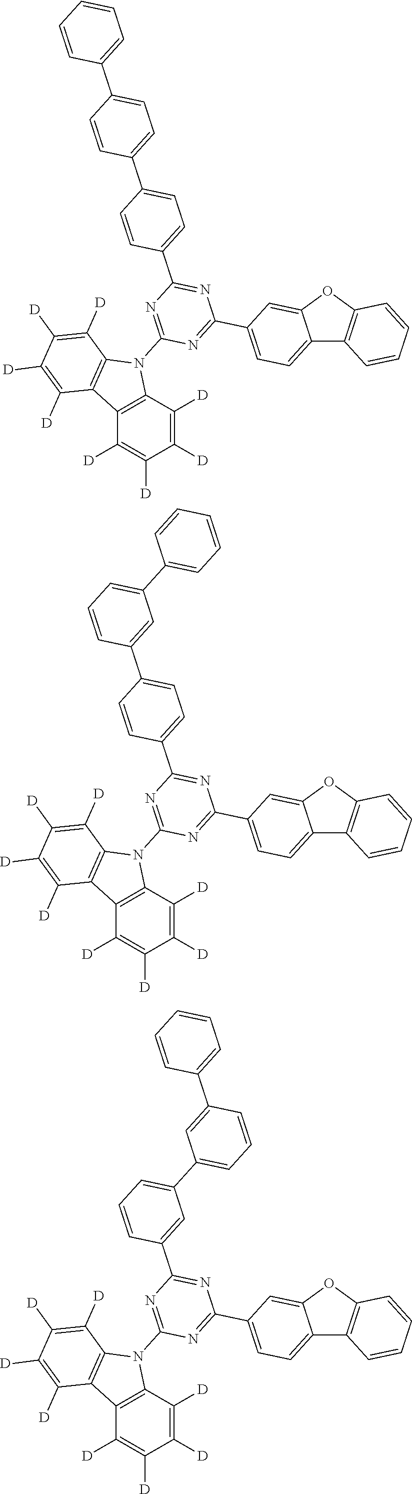

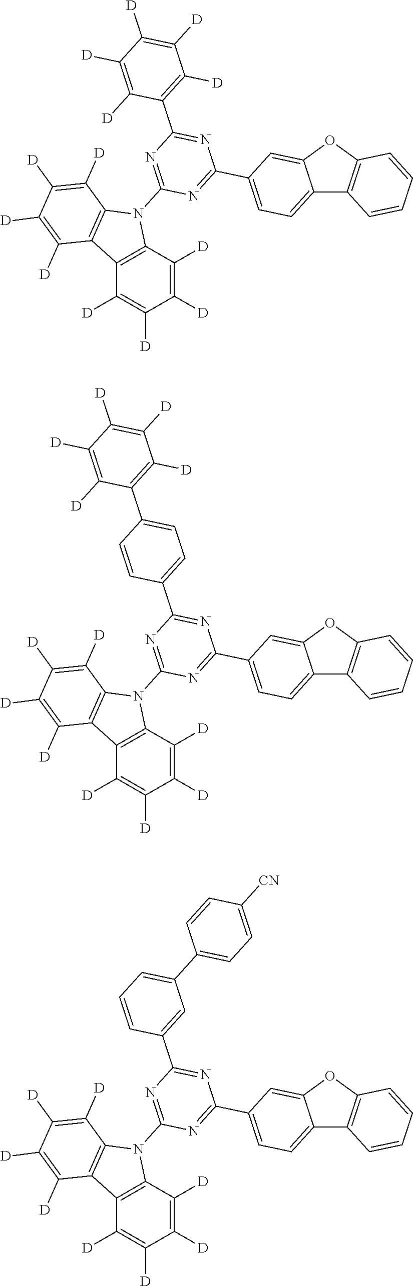 [SMILES: [2H]c1c([2H])c([2H])c(-c2nc(-c3ccc4c(c3)oc3ccccc34)nc(-n3c4c([2H])c([2H])c([2H])c([2H])c4c4c([2H])c([2H])c([2H])c([2H])c43)n2)c([2H])c1[2H] ; [2H]c1c([2H])c([2H])c(-c2ccc(-c3nc(-c4ccc5c(c4)oc4ccccc45)nc(-n4c5c([2H])c([2H])c([2H])c([2H])c5c5c([2H])c([2H])c([2H])c([2H])c54)n3)cc2)c([2H])c1[2H] ; [2H]c1c([2H])c([2H])c2c(c1[2H])c1c([2H])c([2H])c([2H])c([2H])c1n2-c1nc(-c2cccc(-c3ccc(C#N)cc3)c2)nc(-c2ccc3c(c2)oc2ccccc23)n1]

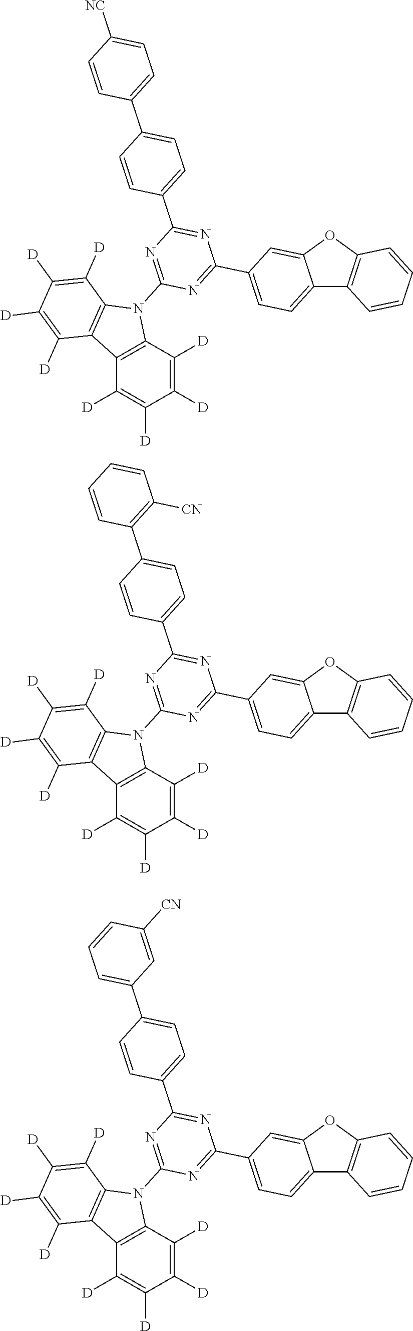

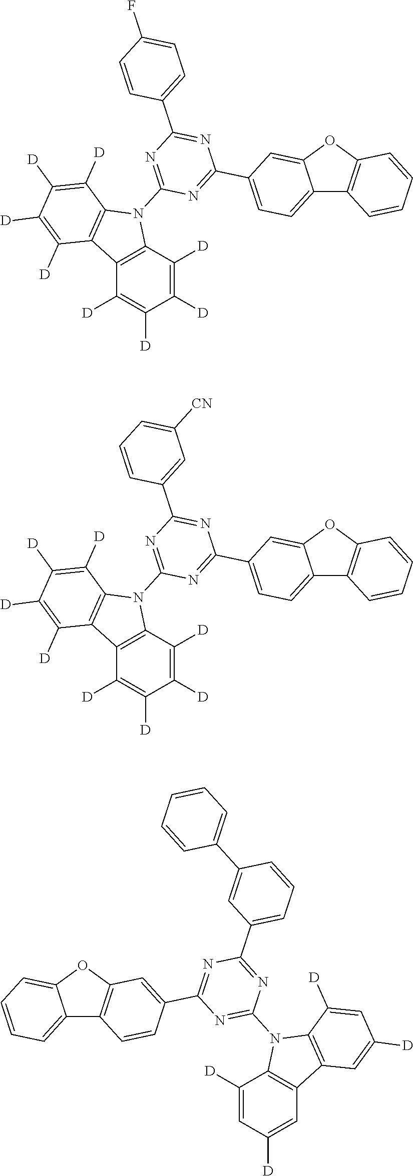 [SMILES: [2H]c1c([2H])c([2H])c2c(c1[2H])c1c([2H])c([2H])c([2H])c([2H])c1n2-c1nc(-c2ccc(F)cc2)nc(-c2ccc3c(c2)oc2ccccc23)n1 ; [2H]c1c([2H])c([2H])c2c(c1[2H])c1c([2H])c([2H])c([2H])c([2H])c1n2-c1nc(-c2cccc(C#N)c2)nc(-c2ccc3c(c2)oc2ccccc23)n1 ; [2H]c1cc([2H])c2c(c1)c1cc([2H])cc([2H])c1n2-c1nc(-c2cccc(-c3ccccc3)c2)nc(-c2ccc3c(c2)oc2ccccc23)n1]

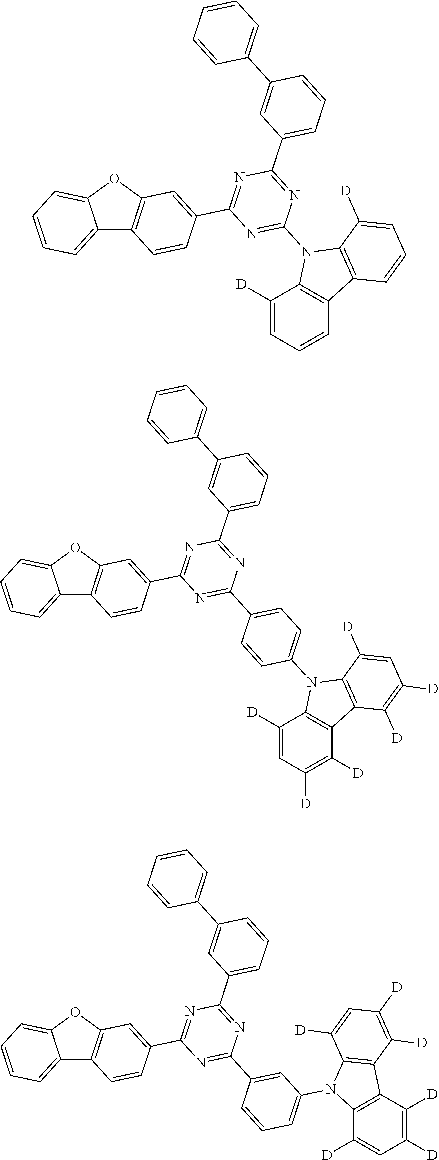 [SMILES: [2H]c1cccc2c3cccc([2H])c3n(-c3nc(-c4cccc(-c5ccccc5)c4)nc(-c4ccc5c(c4)oc4ccccc45)n3)c12 ; [2H]c1cc([2H])c2c(c1[2H])c1c([2H])c([2H])cc([2H])c1n2-c1ccc(-c2nc(-c3cccc(-c4ccccc4)c3)nc(-c3ccc4c(c3)oc3ccccc34)n2)cc1 ; [2H]c1cc([2H])c2c(c1[2H])c1c([2H])c([2H])cc([2H])c1n2-c1cccc(-c2nc(-c3cccc(-c4ccccc4)c3)nc(-c3ccc4c(c3)oc3ccccc34)n2)c1]

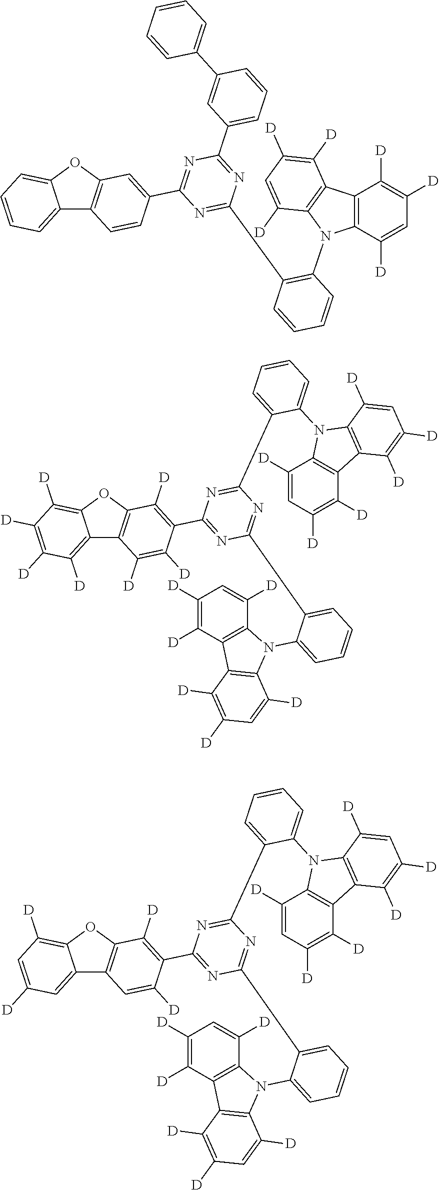

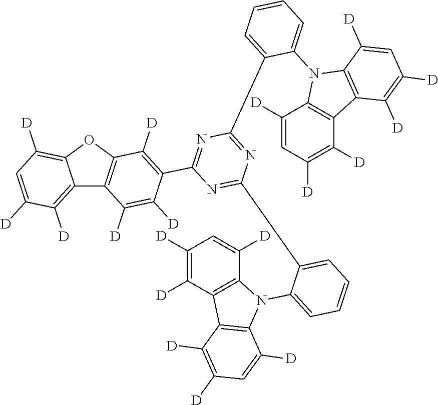 [SMILES: [2H]c1cc([2H])c2oc3c([2H])c(-c4nc(-c5ccccc5-n5c6c([2H])cc([2H])c([2H])c6c6c([2H])c([2H])cc([2H])c65)nc(-c5ccccc5-n5c6c([2H])cc([2H])c([2H])c6c6c([2H])c([2H])cc([2H])c65)n4)c([2H])c([2H])c3c2c1[2H]]

10. An organic light emitting device comprising:
a first electrode;
a second electrode provided to face the first electrode; and
an organic material layer including one or more layers provided between the first electrode and the second electrode,
wherein one or more layers of the organic material layer comprise the compound as set forth in claim 1.

11. The organic light emitting device of claim 10, wherein
the organic material layer comprises a light emitting layer, and the light emitting layer comprises the compound.
